(19) United States
(12) Patent Application Publication
(10) Pub. No.: US 2019/0328838 A1
(43) Pub. Date: Oct. 31, 2019

(54) COMPOSITIONS COMPRISING IL-2 FUSION PROTEINS AND METHODS FOR TREATING NEOPLASIA

(71) Applicant: Altor Bioscience, LLC, Miramar, FL (US)

(72) Inventors: Jinghai Wen, Weston, FL (US); Wenxin Xu, Pembroke Pines, FL (US); Peter Rhode, Miami, FL (US); Hing C. Wong, Weston, FL (US)

(21) Appl. No.: 16/510,835

(22) Filed: Jul. 12, 2019

Related U.S. Application Data
(63) Continuation of application No. 14/388,575, filed on Sep. 26, 2014.
(60) Provisional application No. 61/617,370, filed on Mar. 29, 2012.

(51) Int. Cl. A61K 38/20; A61K 38/21; A61K 38/17; A61K 45/06; A61K 38/08; A61K 31/7068; A61K 33/24

(52) U.S. Cl. CPC A61K 38/2013; A61K 38/212; A61K 38/1774; C07K 2319/32; A61K 38/08; A61K 31/7068; A61K 33/24; A61K 45/06

(57) ABSTRACT

The invention provides methods of treating neoplasia, for example bladder cancer, by administering an IL-2 fusion protein and one or more therapeutic agents, where the IL-2 fusion protein does not necessarily have to target the neoplasia.

### STATEMENT OF RIGHTS TO INVENTIONS MADE UNDER FEDERALLY SPONSORED RESEARCH

This work was supported by the following grants from the National Institutes of Health, Grant No: CA097550. The government has certain rights in the invention.

### BACKGROUND OF THE INVENTION

In the United States, bladder cancer (also referred to herein as urothelial cancer) is the fourth most common type of cancer in men and the ninth most common cancer in women, with an estimated 70,500 new cases (52,760 men and 17,770 women) and 14,680 deaths (10,410 men and 4,270 women) annually (Jemal, A. et al., CA Cancer J Clin, 60: 277-300, 2010). Localized disease is often treated using immunotherapy (Bacillus Calmette-Guerin), an electrocautery device connected to a cytoscope, or by cystectomy. Advanced disease is often treated by chemotherapy or a combination of chemotherapy and radiation. For metastatic muscle-invasive bladder cancer patients treated with conventional single-agent chemotherapy, the median survival is approximately 7 to 8 months (Raghavan, D. et al., N Engl J Med, 322: 1129-1138, 1990). With the introduction of combination cytotoxic regimens including methotrexate, vinblastine, doxorubicin, and cisplatin (MVAC) and gemcitabine and cisplatin (GC) to the management of metastatic bladder cancer, median survival figures have nearly doubled to over 13 months, with a 3-year survival of approximately 20% to 25% (Loehrer, P. J. et al., J Clin Oncol, 10: 1066-1073, 1992; von der Maase, H. et al., J Clin Oncol, 18: 3068-3077, 2000). Nevertheless, death from cancer ultimately occurs in more than 90% of such cases and no new drugs for advanced/metastatic bladder cancer have been approved in the last 20 years. Given the limited efficacy of current treatment options, additional therapeutic modalities are needed.

### SUMMARY OF THE INVENTION

As described below, the present invention features methods of treating cancer. In preferred embodiments the invention features administering an IL-2 fusion protein in combination with one or more therapeutic agents to a subject having cancer in an effective amount to treat the cancer.

In one aspect, the invention generally features a method of ameliorating cancer in a subject involving administering an effective amount of an IL-2 fusion protein and one or more therapeutic agents to the subject in need thereof, thereby ameliorate the cancer.

In anther aspect the invention features a method of reducing tumor burden in a subject involving administering an effective amount of an IL-2 fusion protein and a therapeutic agent to the subject in need thereof, thereby reducing the tumor volume.

In yet another aspect the invention features a method of treating chemo-resistant cancer in a subject involving administering an effective amount of an IL-2 fusion protein and a therapeutic agent to the subject in need thereof, thereby treating the chemo-resistant cancer.

In further aspects the invention features a method of inducing a durable immunological memory response against cancer in a subject involving administering an effective amount of an IL-2 fusion protein and a therapeutic agent to the subject in need thereof, thereby inducing a durable immunological memory response against cancer.

In yet another aspect the invention features a method of increasing the survival of a subject having cancer involving administering an effective amount of an IL-2 fusion protein and a therapeutic agent to the subject in need thereof, thereby increasing the survival of the subject.

In another aspect the invention features a kit for the treatment of bladder cancer containing an IL-2 fusion protein and one or more therapeutic agents.

In various embodiments of any of the above aspects or any other aspects of the invention delineated herein, the IL-2 fusion protein does not specifically target or bind to the cancer. In another embodiment the IL-2 fusion protein comprises a T cell receptor (TCR) domain. In yet another embodiment the T cell receptor domain is a single chain T cell receptor. In further embodiments the one or more therapeutic agents are selected from the group consisting of abiraterone acetate, altretamine, anhydrovinblastine, auristatin, azacitidin, AZD 8477, bendamustin, bevacizumab, bexarotene, bicalutamide, BMS184476, 2,3,4,5,6-pentafluoro-N-(3-fluoro-4-methoxyphenyl)benzene sulfonamide, bleomycin, bortezomib, N,N-dimethyl-L-valyl-L-valyl-N-methyl-L-valyl-L-proly-1-Lproline-t-butylamide, cachectin, capecitabin, cemadotin, cetuximab, chlorambucil, cyclophosphamide, 3′,4′ didehydro 4′ deoxy-8′-norvin-caleukoblastine, docetaxol, doxetaxel, cyclophosphamide, carboplatin, carmustine (BCNU), cisplatin, cryptophycin, cyclophosphamide, cytarabine, dacarbazine (DTIC), dactinomycin, dasatinib, daunorubicin, dolastatin, dovitinib, doxorubicin (adriamycin), epirubicin, epothilone B, erlotinib, eribulin, etoposide, everolimus, 5-fluorouracil, finasteride, flutamide, gefitinib, gemcitabine, hydroxyurea and hydroxyureataxanes, ifosfamide, interferon alfa, imatinib, ipilimumab, irinotecan, largotaxel, lapatinib, lenalidomid, liarozole, lonafarnib, lonidamine, lomustine (CCNU), mechlorethamine (nitrogen mustard), melphalan, mivobulin isethionate, rhizoxin, sertenef, streptozocin, mitomycin, methotrexate, 5-fluorouracil, nilutamide, onapristone, oxaliplatin, paclitaxel, panitumumab, pazopanib, pralatrexate, prednimustine, piritrexim, procarbazine, pyrazoloacridine, rituximab, RPR109881, romidepsin, sorafinib, stramustine phosphate, sunitinib, tamoxifen, tasonermin, taxol, temozolomide, topotecan, transtuzumab, tretinoin, trimetrexate, vemurafenib, vinblastine, vincristine, vindesine sulfate, vinflunine, and vorinostat. In other embodiments the one or more therapeutic agents are selected from the group consisting of gemcitabine and platinum-based compounds including cisplatin. In yet another embodiment the cancer is selected from the group consisting of bladder cancer, urothelial cancer of the urethra, ureter and renal pelvis, kidney cancer, breast cancer, colon cancer, head and neck cancer, lung cancer, prostate cancer, glioblastoma, osteosarcoma, liposarcoma, soft-tissue sarcoma, ovarian cancer, melanoma, liver cancer, esophageal cancer, pancreatic cancer and stomach cancer. In a further embodiment the cancer is bladder or urothelial cancer. In yet further embodiments the cancer is chemo-resistant. In other embodiments the IL-2 fusion protein and the one or more therapeutic agents are administered within about 7-14 days. In yet other embodiments the IL-2 fusion protein and the one or more therapeutic agents are administered within about 3-5 days or are administered concurrently. In additional embodiments the IL-2 fusion protein is ALT-801 and the one or more therapeutic agents is cisplatin. In further embodiments the one or more therapeutic agents is gemcitabine. In yet additional embodiments the IL-2 fusion protein specifically targets the cancer cells. In some embodiments the IL-2 fusion protein specifically targets p53 peptide/HLA complexes on the surface of the cancer cells.

Compositions and articles defined by the invention were isolated or otherwise manufactured in connection with the examples provided below. Other features and advantages of the invention will be apparent from the detailed description, and from the claims.

### Definitions

By “tumor burden” also called “tumor load” is meant the number of cancer cells, the size of a tumor, or the amount of cancer in the body.

By “IL-2 fusion protein” is meant a polypeptide that contains the entire full length IL-2 protein or a biologically active fragment thereof fused to a second polypeptide. The second polypeptide may be a targeting polypeptide, i.e., an antibody or antigen binding fragment thereof; a T cell receptor (TCR) or a peptide binding fragment thereof; a receptor or a ligand binding domain thereof; etc., wherein the second polypeptide specifically targets or directs the IL-2 fusion protein to a cancer cell. Alternatively, the second polypeptide can be a non-targeting polypeptide, i.e., a polypeptide that does not specifically target or direct the IL-2 fusion protein to a cancer cell.

By “T cell receptor (TCR) domain” is meant a polypeptide that comprises all of the portions of a T cell receptor necessary to bind the cognate peptide presented in the appropriate MHC or HLA molecule. Non-limiting examples of TCR domains are described in U.S. Pat. Nos. 7,456,263; 6,534,633; U.S. Patent Application Publication No. US2003/0144474; and U.S. Patent Application Publication No. US2011/0070191, which are incorporated by reference herein in their entirety.

By “ALT-801” is meant a fusion between IL-2 and a TCR domain capable of binding human p53 peptide (aa 264-272) HLA-A*0201 (c264scTCR-IL-2). An illustrative amino acid sequence of ALT-801, including the signal sequence, is:

Metdtlllwvlllwvpgstgqsvtqpdarvtvsegaslqlrckysysgtp
ylfwyvqyprqglqlllkyysgdpvvqgvngfeaefsksnssfhlrkasv
hwsdsavyfcvlsedsnyqliwgsgtkliikpdtsggggsggggsggggs
ggggsssnskviqtprylvkgqgqkakmrcipekghpvvfwyqqnknnef
kflinfqnqevlqqidmtekrfsaecpsnspcsleiqsseagdsalylca
sslsgggtevffgkgtrltvvedlnkvfppevavfepseaeishtqkatl
vclatgffpdhvelswwvngkevhsgvstdpqplkeqpalndsryclssr
lrvsatfwqnprnhfrcqvqfyglsendewtqdrakpvtqivsaeawgra
dvnakttapsvyplapvsgaptssstkktqlqlehllldlqmilnginny
knpkltrmltfkfympkkatelkhlqcleeelkpleevlnlaqsknfhlr
prdlisninvivlelkgsettfmceyadetativeflnrwitfcqsiist
lt

An illustrative amino acid sequence of mature ALT-801, without the signal sequence, is:

qsvtqpdarvtvsegaslqlrckysysgtpylfwyvqyprqglqlllky
ysgdpvvqgvngfeaefsksnssfhlrkasvhwsdsavyfcvlsedsny
qliwgsgtkliikpdtsggggsggggsggggsggggsssnskviqtpry
lvkgqgqkakmrcipekghpvvfwyqqnknnefkflinfqnqevlqqid
mtekrfsaecpsnspcsleiqsseagdsalylcasslsgggtevffgkg
trltvvedlnkvfppevavfepseaeishtqkatlvclatgffpdhvel
swwvngkevhsgvstdpqplkeqpalndsryclssrlrvsatfwqnprn
hfrcqvqfyglsendewtqdrakpvtqivsaeawgradvnakttapsvy
plapvsgaptssstkktqlqlehllldlqmilnginnyknpkltrmltf
kfympkkatelkhlqcleeelkpleevlnlaqsknfhlrprdlisninv
ivlelkgsettfmceyadetativeflnrwitfcqsiistlt

An illustrative nucleic acid encoding ALT-801 is:

atggagacagacacactcctgttatgggtactgctgctctgggttccag
gttccaccggtcagtcagtgacgcagcccgatgctcgcgtcactgtctc
tgaaggagcctctctgcagctgagatgcaagtattcctactctgggaca
ccttatctgttctggtatgtccagtacccgcggcaggggctgcagctgc
tcctcaagtactattcaggagacccagtggttcaaggagtgaatggctt
cgaggctgagttcagcaagagtaactcttccttccacctgcggaaagcc
tctgtgcactggagcgactctgctgtgtacttctgtgttttgagcgagg
atagcaactatcagttgatctggggctctgggaccaagctaattataaa
gccagacactagtggtggcggtggcagcggcggtggtggttccggtggc
ggcggttctggcggtggcggttcctcgagcaattcaaaagtcattcaga
ctccaagatatctggtgaaagggcaaggacaaaaagcaaagatgaggtg
tatccctgaaaagggacatccagttgtattctggtatcaacaaaataag
aacaatgagtttaaatttttgattaactttcagaatcaagaagttcttc
agcaaatagacatgactgaaaaacgattctctgctgagtgtccttcaaa
ctcaccttgcagcctagaaattcagtcctctgaggcaggagactcagca
ctgtacctctgtgccagcagtctgtcagggggcggcacagaagttttct
ttggtaaaggaaccagactcacagttgtagaggacctgaacaaggtgtt
cccacccgaggtcgctgtgtttgagccatcagaagcagagatctcccac
acccaaaaggccacactggtgtgcctggccacaggcttottccctgacc
acgtggagctgagctggtgggtgaatgggaaggaggtgcacagtggggt
cagcacggacccgcagcccctcaaggagcagcccgccctcaatgactcc
agatactgcctgagcagccgcctgagggtctcggccaccttctggcaga
acccccgcaaccacttccgctgtcaagtccagttctacgggctctcgga
gaatgacgagtggacccaggatagggccaaacccgtcacccagatcgtc
agcgccgaggcctggggtagagcagacgttaacgcaaagacaaccgccc
cttcagtatatccactagcgcccgtttccggagcacctacttcaagttc
tacaaagaaaacacagctacaactggagcatttactgctggatttacag
atgattttgaatggaattaataattacaagaatcccaaactcaccagga
tgctcacatttaagttttacatgcccaagaaggccacagaactgaaaca
tcttcagtgtctagaagaagaactcaaacctctggaggaagtgctaaat
ttagctcaaagcaaaaactttcacttaagacccagggacttaatcagca
atatcaacgtaatagttctggaactaaagggatctgaaacaacattcat
gtgtgaatatgctgatgagacagcaaccattgtagaatttctgaacaga
tggattaccttttgtcaaagcatcatctcaacactaacttaa

By “MART-1scTCR/IL-2” is meant a fusion between IL-2 and a TCR domain capable of binding MART-1 peptide (aa 27-35) presented in the context of HLA-A*0201. An illustrative amino acid sequence of MART-1scTCR/IL-2, including the signal sequence, is:

Metdtlllwvlllwvpgstgqkevegnsgplsvpegaiaslnctysdrg
sqsffwyrqysgkspelimfiysngdkedgrftaqlnkasqyvsllird
sqpsdsatylcavnfgggklifgqgtelsvkpdtsggggsgggasgggg
sggggsssiagitqaptsqilaagrrmtlrctqdmrhnamywyrqdlgl
glrlihysntagttgkgevpdgysysrantddfpltlasavpsqtsvyf
casslsfgteaffgqgtrltvvedlnkvfppevavfepseaeishtqka
tlvclatgffpdhvelswwvngkevhsgvstdpqplkeqpalndsrycl
ssrlrvsatfwqnprnhfrcqvqfyglsendewtqdrakpvtqivsaea
wgradvnakttapsvyplapvsgaptssstkktqlqlehllldlqmiln
ginnyknpkltrmltfkfympkkatelkhlqcleeelkpleevlnlaqs
knfhlrprdlisninvivlelkgsettfmceyadetativeflnrwitf
cqsiistlt

An illustrative amino acid sequence of mature MART-1scTCR/IL-2, without the signal sequence, is:

Qkeveqnsgplsvpegaiaslnctysdrgsqsffwyrqysgkspelimfi
ysngdkedgrftaqlnkasqyvsllirdsqpsdsatylcavnfgggklif
gqgtelsvkpdtsggggsgggasggggsggggsssiagitqaptsqilaa
grrmtlrctqdmrhnamywyrqdlglglrlihysntagttgkgevpdgys
ysrantddfpltlasavpsqtsvyfcasslsfgteaffgqgtrltvvedl
nkvfppevavfepseaeishtqkatlvclatgffpdhvelswwvngkevh
sgvstdpqplkeqpalndsryclssrlrvsatfwqnprnhfrcqvqfygl
sendewtqdrakpvtqivsaeawgradvnakttapsvyplapvsgaptss
stkktqlqlehllldlqmilnginnyknpkltrmltfkfympkkatelkh
lqcleeelkpleevlnlaqsknfhlrprdlisninvivlelkgsettfmc
eyadetativeflnrwitfcqsiistlt

An illustrative nucleic acid encoding MART-1scTCR/IL-2 is:

atggagacagacacactcctgttatgggtactgctgctctgggttccagg
ttccaccggtcagaaggaggtggagcagaattctggacccctcagtgttc
cagagggagccattgcctctctcaactgcacttacagtgaccgaggttcc
cagtccttcttctggtacagacaatattctgggaaaagccctgagttgat
aatgttcatatactccaatggtgacaaagaagatggaaggtttacagcac
agctcaataaagccagccagtatgtttctctgctcatcagagactcccag
cccagtgattcagccacctacctctgtgccgtgaacttcggaggaggaaa
gcttatcttcggacagggaacggagttatctgtgaaacccgacactagtg
gtgggggtgggagcgggggtggtgctagcggtggcggcggttctggcggt
ggcggttcctccagcattgcagggatcacccaggcaccaacatctcagat
cctggcagcaggacggcgcatgacactgagatgtacccaggatatgagac
ataatgccatgtactggtatagacaagatctaggactggggctaaggctc
atccattattcaaatactgcaggtaccactggcaaaggagaagtccctga
tggttatagtgtctccagagcaaacacagatgatttccccctcacgttgg
cgtctgctgtaccctctcagacatctgtgtacttctgtgccagcagccta
agtttcggcactgaagctttctttggacaaggcaccagactcacagttgt
agaggacctgaacaaggtgttcccacccgaggtcgctgtgtttgagccat
cagaagcagagatctcccacacccaaaaggccacactggtgtgcctggcc
acaggcttcttccctgaccacgtggagctgagctggtgggtgaatgggaa
ggaggtgcacagtggggtcagcacggacccgcagcccctcaaggagcagc
ccgccctcaatgactccagatactgcctgagcagccgcctgagggtctcg
gccaccttctggcagaacccccgcaaccacttccgctgtcaagtccagtt
ctacgggctctcggagaatgacgagtggacccaggatagggccaaacccg
tcacccagatcgtcagcgccgaggcctggggtagagcagacgttaacgca
aagacaaccgccccttcagtatatccactagcgcccgtttccggagcacc
tacttcaagttctacaaagaaaacacagctacaactggagcatttactgc
tggatttacagatgattttgaatggaattaataattacaagaatcccaaa
ctcaccaggatgctcacatttaagttttacatgcccaagaaggccacaga
actgaaacatcttcagtgtctagaagaagaactcaaacctctggaggaag
tgctaaatttagctcaaagcaaaaactttcacttaagacccagggactta
atcagcaatatcaacgtaatagttctggaactaaagggatctgaaacaac
attcatgtgtgaatatgctgatgagacagcaaccattgtagaatttctga
acagatggattaccttttgtcaaagcatcatctcaacactaactta.

By “agent” is meant any small molecule chemical compound, antibody, nucleic acid molecule, or polypeptide, or fragments thereof.

By “therapeutic agent” is meant any chemotherapeutic or biotherapeutic agent that is used in the treatment of cancer. Non-limiting illustrative examples of therapeutic agents include abiraterone acetate, altretamine, anhydrovinblastine, auristatin, azacitidin, AZD 8477, bendamustin, bevacizumab, bexarotene, bicalutamide, BMS184476, 2,3,4,5,6-pentafluoro-N-(3-fluoro-4-methoxyphenyl)benzene sulfonamide, bleomycin, bortezomib, N,N-dimethyl-L-valyl-L-valyl-N-methyl-L-valyl-L-proly-1-Lproline-t-butylamide, cachectin, capecitabin, cemadotin, cetuximab, chlorambucil, cyclophosphamide, 3′,4′-didehydro-4′-deoxy-8′-norvin-caleukoblastine, docetaxol, doxetaxel, cyclophosphamide, carboplatin, carmustine (BCNU), cisplatin, cryptophycin, cyclophosphamide, cytarabine, dacarbazine (DTIC), dactinomycin, dasatinib, daunorubicin, dolastatin, dovitinib, doxorubicin (adriamycin), epirubicin, epothilone B, erlotinib, eribulin, etoposide, everolimus, 5-fluorouracil, finasteride, flutamide, gefitinib, gemcitabine, hydroxyurea and hydroxyureataxanes, ifosfamide, interferon alfa, imatinib, ipilimumab, irinotecan, largotaxel, lapatinib, lenalidomid, liarozole, lonafarnib, lonidamine, lomustine (CCNU), mechlorethamine (nitrogen mustard), melphalan, mivobulin isethionate, rhizoxin, sertenef, streptozocin, mitomycin, methotrexate, 5-fluorouracil, nilutamide, onapristone, oxaliplatin, paclitaxel, panitumumab, pazopanib, pralatrexate, prednimustine, piritrexim, procarbazine, pyrazoloacridine, rituximab, RPR109881, romidepsin, sorafinib, stramustine phosphate, sunitinib, tamoxifen, tasonermin, taxol, temozolomide, topotecan, transtuzumab, tretinoin, trimetrexate, vemurafenib, vinblastine, vincristine, vindesine sulfate, vinflunine, and vorinostat.

By “chemo-resistant” is meant a cancer or cancer cell that has become resistant to one or more therapeutic agents.

By “ameliorate” is meant decrease, suppress, attenuate, diminish, arrest, or stabilize the development or progression of a disease.

By “inducing a durable immunological memory response against tumors” is meant treatment-induced resistance to subsequent challenge or regrowth of a tumor or cancerous growth.

By “alteration” is meant a change (increase or decrease) in the expression levels or activity of a gene or polypeptide as detected by standard art known methods such as those described herein. As used herein, an alteration includes a 10% change in expression levels, preferably a 25% change, more preferably a 40% change, and most preferably a 50% or greater change in expression levels.”

By “analog” is meant a molecule that is not identical, but has analogous functional or structural features. For example, a polypeptide analog retains the biological activity of a corresponding naturally-occurring polypeptide, while having certain biochemical modifications that enhance the analog's function relative to a naturally occurring polypeptide. Such biochemical modifications could increase the analog's protease resistance, membrane permeability, or half-life, without altering, for example, ligand binding. An analog may include an unnatural amino acid.

In this disclosure, “comprises,” “comprising,” “containing” and “having” and the like can have the meaning ascribed to them in U.S. patent law and can mean “includes,” “including,” and the like; “consisting essentially of” or “consists essentially” likewise has the meaning ascribed in U.S. patent law and the term is open-ended, allowing for the presence of more than that which is recited so long as basic or novel characteristics of that which is recited is not changed by the presence of more than that which is recited, but excludes prior art embodiments.

“Detect” refers to identifying the presence, absence or amount of the analyte to be detected.

By “detectable label” is meant a composition that when linked to a molecule of interest renders the latter detectable, via spectroscopic, photochemical, biochemical, immunochemical, or chemical means. For example, useful labels include radioactive isotopes, magnetic beads, metallic beads, colloidal particles, fluorescent dyes, electron-dense reagents, enzymes (for example, as commonly used in an ELISA), biotin, digoxigenin, or haptens.

By “disease” is meant any condition or disorder that damages or interferes with the normal function of a cell, tissue, or organ. Examples of diseases include cancer.

By “effective amount” or “therapeutic amount” is meant the amount of a required to treat, prevent or ameliorate the symptoms of a disease relative to an untreated patient. The effective amount of active compound(s) used to practice the present invention for therapeutic treatment of a disease varies depending upon the manner of administration, the age, body weight, and general health of the subject. Ultimately, the attending physician or veterinarian will decide the appropriate amount and dosage regimen. Such amount is referred to as an “effective” amount.

By “fragment” is meant a portion of a polypeptide or nucleic acid molecule. This portion contains, preferably, at least 10%, 20%, 30%, 40%, 50%, 60%, 70%, 80%, or 90% of the entire length of the reference nucleic acid molecule or polypeptide. A fragment may contain 10, 20, 30, 40, 50, 60, 70, 80, 90, or 100, 200, 300, 400, 500, 600, 700, 800, 900, or 1000 nucleotides or amino acids.

“Hybridization” means hydrogen bonding, which may be Watson-Crick, Hoogsteen or reversed Hoogsteen hydrogen bonding, between complementary nucleobases. For example, adenine and thymine are complementary nucleobases that pair through the formation of hydrogen bonds.

By “isolated polynucleotide” is meant a nucleic acid (e.g., a DNA) that is free of the genes which, in the naturally-occurring genome of the organism from which the nucleic acid molecule of the invention is derived, flank the gene. The term therefore includes, for example, a recombinant DNA that is incorporated into a vector; into an autonomously replicating plasmid or virus; or into the genomic DNA of a prokaryote or eukaryote; or that exists as a separate molecule (for example, a cDNA or a genomic or cDNA fragment produced by PCR or restriction endonuclease digestion) independent of other sequences. In addition, the term includes an RNA molecule that is transcribed from a DNA molecule, as well as a recombinant DNA that is part of a hybrid gene encoding additional polypeptide sequence.

By an “isolated polypeptide” is meant a polypeptide of the invention that has been separated from components that naturally accompany it. Typically, the polypeptide is isolated when it is at least 60%, by weight, free from the proteins and naturally-occurring organic molecules with which it is naturally associated. Preferably, the preparation is at least 75%, more preferably at least 90%, and most preferably at least 99%, by weight, a polypeptide of the invention. An isolated polypeptide of the invention may be obtained, for example, by extraction from a natural source, by expression of a recombinant nucleic acid encoding such a polypeptide; or by chemically synthesizing the protein. Purity can be measured by any appropriate method, for example, column chromatography, polyacrylamide gel electrophoresis, or by HPLC analysis.

By “marker” is meant any protein or polynucleotide having an alteration in expression level or activity that is associated with a disease or disorder.

As used herein, “obtaining” as in “obtaining an agent” includes synthesizing, purchasing, or otherwise acquiring the agent.

“Primer set” means a set of oligonucleotides that may be used, for example, for PCR. A primer set would consist of at least 2, 4, 6, 8, 10, 12, 14, 16, 18, 20, 30, 40, 50, 60, 80, 100, 200, 250, 300, 400, 500, 600, or more primers.

As used herein, “recombinant” includes reference to a polypeptide produced using cells that express a heterologous polynucleotide encoding the polypeptide. The cells produce the recombinant polypeptide because they have been genetically altered by the introduction of the appropriate isolated nucleic acid sequence. The term also includes reference to a cell, or nucleic acid, or vector, that has been modified by the introduction of a heterologous nucleic acid or the alteration of a native nucleic acid to a form not native to that cell, or that the cell is derived from a cell so modified. Thus, for example, recombinant cells express genes that are not found within the native (non-recombinant) form of the cell, express mutants of genes that are found within the native form, or express native genes that are otherwise abnormally expressed, under-expressed or not expressed at all.

By “reduces” is meant a negative alteration of at least 10%, 25%, 50%, 75%, or 100%.

By “reference” is meant a standard or control condition.

A “reference sequence” is a defined sequence used as a basis for sequence comparison. A reference sequence may be a subset of or the entirety of a specified sequence; for example, a segment of a full-length cDNA or gene sequence, or the complete cDNA or gene sequence. For polypeptides, the length of the reference polypeptide sequence will generally be at least about 16 amino acids, preferably at least about 20 amino acids, more preferably at least about 25 amino acids, and even more preferably about 35 amino acids, about 50 amino acids, or about 100 amino acids. For nucleic acids, the length of the reference nucleic acid sequence will generally be at least about 50 nucleotides, preferably at least about 60 nucleotides, more preferably at least about 75 nucleotides, and even more preferably about 100 nucleotides or about 300 nucleotides or any integer thereabout or therebetween.

By “specifically binds” is meant a fusion protein that recognizes and binds a cancer cell expressing a particular marker, but which does not substantially recognize and bind other cells in a sample.

By “substantially identical” is meant a polypeptide or nucleic acid molecule exhibiting at least 50% identity to a reference amino acid sequence (for example, any one of the amino acid sequences described herein) or nucleic acid sequence (for example, any one of the nucleic acid sequences described herein). Preferably, such a sequence is at least 60%, more preferably 80% or 85%, and more preferably 90%, 95% or even 99% identical at the amino acid level or nucleic acid to the sequence used for comparison.

Sequence identity is typically measured using sequence analysis software (for example, Sequence Analysis Software Package of the Genetics Computer Group, University of Wisconsin Biotechnology Center, 1710 University Avenue, Madison, Wis. 53705, BLAST, BESTFIT, GAP, or PILEUP/PRETTYBOX programs). Such software matches identical or similar sequences by assigning degrees of homology to various substitutions, deletions, and/or other modifications. Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine. In an exemplary approach to determining the degree of identity, a BLAST program may be used, with a probability score between e⁻³ and e⁻¹⁰⁰ indicating a closely related sequence.

By “subject” is meant a mammal, including, but not limited to, a human or non-human mammal, such as a bovine, equine, canine, ovine, or feline.

A “tumor,” as used herein, refers to all neoplastic cell growth and proliferation, whether malignant or benign, and all precancerous and cancerous cells and tissues.

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50.

As used herein, the terms “treat,” treating,” “treatment,” and the like refer to reducing or ameliorating a disorder and/or symptoms associated therewith. It will be appreciated that, although not precluded, treating a disorder or condition does not require that the disorder, condition or symptoms associated therewith be completely eliminated.

Unless specifically stated or obvious from context, as used herein, the term “or” is understood to be inclusive. Unless specifically stated or obvious from context, as used herein, the terms “a”, “an”, and “the” are understood to be singular or plural.

Unless specifically stated or obvious from context, as used herein, the term “about” is understood as within a range of normal tolerance in the art, for example within 2 standard deviations of the mean. “About” can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term about.

The recitation of a listing of chemical groups in any definition of a variable herein includes definitions of that variable as any single group or combination of listed groups. The recitation of an embodiment for a variable or aspect herein includes that embodiment as any single embodiment or in combination with any other embodiments or portions thereof.

Any compositions or methods provided herein can be combined with one or more of any of the other compositions and methods provided herein.

### BRIEF DESCRIPTION OF THE DRAWINGS

FIG. 1 is a graph showing changes in mean tumor volume of subcutaneous human UMUC-14 bladder tumor xenografts in nude mice over 40 days with two treatment cycles of gemcitabine+cisplatin; ALT-801; or gemcitabine+cisplatin+ALT-801.

FIG. 2 is a graph showing changes in mean tumor volume of subcutaneous human UMUC-14 bladder tumor xenografts in nude mice over 48 days with two treatment cycles separated by a 11 day rest of gemcitabine+cisplatin; gemcitabine+MART-1scTCR/IL-2; ALT-801; or gemcitabine+ALT-801.

FIG. 3 is a graph showing the effects of ALT-801 and MART-1scTCR/IL-2, in combination with chemotherapy regimens, on growth of subcutaneous human bladder UMUC-14 xenografts in nude mice.

FIG. 4 is a graph showing the effects of ALT-801 and MART-1scTCR/IL-2, in combination with chemotherapy regimens, on mouse body weight.

FIG. 5 is a graph showing the effects of ALT-801 and MART-1scTCR/IL-2, in combination with chemotherapy regimens, on growth of subcutaneous human bladder KU7P xenografts in nude mice.

FIG. 6 is a graph showing the effects of ALT-801 and MART-1scTCR/IL-2, in combination with chemotherapy regimens, on mouse body weight.

FIG. 7 is a graph showing the effects of gemcitabine, ALT-801 and MART-1scTCR/IL-2 on growth of subcutaneous human bladder KU7P xenografts in nude mice.

FIG. 8 is a graph showing the survival of albino C57BL/6 mice harboring orthotopic MB49luc tumors treated with either ALT-801 or PBS (control).

FIG. 9A is a graph showing the survival of C57BL/6 mice harboring orthotopic MB49luc tumors treated with either ALT-801 or PBS (control). FIG. 9B is an image showing bioluminescence of orthotopic MB49luc tumors in treatment naïve or ALT-801 treated C57BL/6 mice.

FIG. 10 is a graph showing the survival of C57BL/6 mice harboring orthotopic MB49luc tumors treated with either ALT-801 or PBS (control).

FIG. 11 is a graph showing the survival of C57BL/6 mice with MB49luc superficial bladder tumors treated with ALT-801.

FIGS. 12A and 12B are graphs showing the survival of C57BL/6 mice with MB49luc superficial bladder tumors treated with ALT-801 once weekly (“1×4”) (FIG. 12A) or twice weekly (“2×4”) (FIG. 12B) for four weeks.

FIG. 13 is images of H&E-stained bladder tissue sections from normal and MB49luc tumor-bearing CS7BL/6 mice following treatment with PBS or ALT-801.

FIGS. 14A and 14B are graphs showing immune cell populations in the PMBCs (FIG. 14A) and spleen (FIG. 14B) from normal and MB49luc tumor-bearing C57BL/6 mice following treatment with PBS or ALT-801.

FIG. 15 is images showing stained macrophages in bladder tissue sections from MB49luc tumor-bearing C57BL/6 mice on study day 10 following treatment with PBS or ALT-801.

FIGS. 16A and 16B are graphs showing changes in macrophage levels in the bladders from normal (FIG. 16A) and MB49luc tumor-bearing C57BL/6 mice (FIG. 16B) following treatment with PBS or ALT-801.

FIGS. 17A and 17B are graphs showing changes in urine IFNγ (FIG. 17A) and TNFα (FIG. 17B) in normal and MB49luc tumor-bearing C57BL/6 mice following treatment with PBS or ALT-801.

FIG. 18 is a graph showing treatment with ALT-801 but not IL-2 prolonged survival of mice bearing orthotopic MB49luc bladder tumors. C57BL/6 mice (10-11 weeks old) were instilled intravesically with MB49luc cells (3×104 cells/bladder) on study day 0, following polylysine pretreatment of the bladders. ALT-801 (1.6 mg/kg, n=8), rIL2 (0.42 mg/kg, n=8) or PBS (100 μL, n=8) was administered i.v. on days 7, 10, 14 and 17 post MB49luc tumor cell instillation. Kaplan-Meier survival curves comparing the study groups are shown.

FIGS. 19A-19D depict the effect of Mø, NK, CD4 and CD8 cell depletion on ALT-801 efficacy in C57BL/6 mice bearing mouse MB49luc orthotopic bladder tumors. FIG. 19A is a graph depicting survival of mice administered ALT-801 compared to mice administered PBS. FIG. 19B is a graph depicting survival of mice administered ALT-801 and subjected to NK cell depletion by i.p. injection of anti-NK antibody (Ab) (clone PK136, 250 μg in 100 μL) on SD 2, 3, 6, 9, 13, and 16, compared to mice administered PBS. FIG. 19C is a graph depicting survival of mice administered ALT-801 and subjected to Mø depletion by i.p. injection of Clophosome (150 μL/dose) on SD 6, 9, 13, and 16, compared to mice administered PBS. FIG. 19D is a graph depicting survival of mice administered ALT-801 and subjected to CD4 and CD8 cell depletion by i.p. injection of anti-CD4 Ab (clone GK1.5, 250 μg in 100 μL) and anti-CD8 Ab (clone 53-6.72, 250 μg in 100 μL) on SD 2, 3, 6, 9, 13, and 16, compared to mice administered PBS. Kaplan-Meier survival plots are displayed. P values ≤0.05 are considered significant.

FIG. 20 is a graph depicting changes in blood MDSC levels in C57BL/6 mice bearing mouse MB49luc orthotopic bladder tumors. Bars represent the mean±SEM. * P ≤0.05 compared to control.

FIG. 21 are images of immunohistochemistry staining of macrophages in mouse bladders bearing MB49luc orthotopic bladder tumors. On SD 0, mice received MB49luc instillation and 11 days later received PBS or ALT-801 (1.6 mg/kg) i.v. treatment. Mice were sacrificed 24 hours after treatment and bladders were collected for staining. Bladder sections were stained with anti-iNOS (M1 macrophage marker), and anti-MMP-9 (M2 macrophage marker) and anti-F4/80 (macrophage pan marker) Abs. Representative tissue sections are shown. Magnification 200×.

FIG. 22 is a graph depicting the role of immune cell subsets in ALT-801-mediated induction of serum IFN-γ levels in C57BL/6 mice. C57BL/6 female mice were injected peritoneally with anti-CD4 (GK1.5), anti-CD8 (53-6.72), and/or anti-NK1.1 (PK136) Abs to deplete immune cell subsets. The mice were then injected intravenously with 1.2 mg/kg ALT-801 and serum IFN-γ levels were determined 24 hours later by ELISA. The bars represent the mean±standard error (n=5/group).

FIG. 23 is a graph depicting the effect of IFN-γ on MB49luc cell growth in vitro. MB49luc cells (2×10⁵/well) were cultured in RPMI-10 with IFN-γ at 1 ng/mL or 10 ng/mL for 2 days. The apoptotic MB49luc cells were determined by flow cytometry following Annexin V staining.

FIG. 24 is a graph depicting that ALT-801 induced LAK cell cytotoxicity against MB49luc tumor cells. Lymphokine activated killer (LAK) cells were prepared from mouse splenocytes following in vitro activation by 20 nM ALT-801 for 3 days. The LAK cells (4×10⁶/well) were cultured with PKH67-labeled MB49luc (4×10⁵/well) in RPMI-10 with 0 to 50 nM ALT-801. The cultured cells were harvested 24 hours later and labeled with 0.001 mg/mL PI. The percentage of dead Pr MB49luc cells was determined by flow cytometry.

FIG. 25 is a graph depicting that gemcitabine reduced splenocyte MDSC levels in MB49luc tumor bearing mice. Female C57BL/6 mice were injected intravenously with MB49luc cells (1×106/mouse). After 10 days, one group of mice was treated intravenously 40 mg/kg gemcitabine. Mice were sacrificed 3 days later and the splenocytes were isolated. The percentage of spleen Gr1+CD11b+ MDSCs was determined by flow cytometry.

FIG. 26 depicts flow cytometry analysis of MDSC purity post magnetic sorting. Cells positively selected by MACS columns were stained with anti-CD11b-PE and anti-Gr1-FITC antibodies. CD11b+Gr1+ cells later subjected to adoptive transfer had a purity of 96%.

FIG. 27 is a graph depicting that ALT-801 induced tumor cell killing by immune cells after MDSC adoptive transfer. Splenocytes from MDSC recipient mice (black) or vehicle control mice (white) were collected and activated into LAK cells by incubation with 50 nM ALT-801. LAK effector cells were then mixed with MB49luc target cells to assess their cytolytic activity. Data from fresh spleen cells without ALT-801 activation, as well as cytolytic activity assessed following addition of ALT-801 during killing phase are also plotted. ***: P<0.001. n=2.

FIG. 28 depicts a study design and treatment scheme for a Phase I/II clinical trial of ALT-801 administered in combination with gemcitabine and cisplatin in urothelial cancer.

FIG. 29 depicts a study design and treatment scheme for a Phase I/II clinical trial of ALT-801 administered in combination with gemcitabine and cisplatin in urothelial cancer.

FIG. 30 depicts patient demographics and disease status of a Phase I/II clinical trial of ALT-801 administered in combination with gemcitabine and cisplatin in urothelial cancer.

FIG. 31 depicts tumor assessment in a Phase I/II clinical trial of ALT-801 administered in combination with gemcitabine and cisplatin in urothelial cancer.

FIG. 32 depicts objective responses in patients administered ALT-801 in a Phase I/II clinical trial of ALT-801 administered in combination with gemcitabine and cisplatin in urothelial cancer.

FIG. 33 depicts progression free survival in patients administered ALT-801 in a Phase I/II clinical trial of ALT-801 administered in combination with gemcitabine and cisplatin in urothelial cancer.

FIG. 34 are graphs depicting increased serum IFN-γ levels in patients administered ALT-801 (left panel: 0.04 mg/kg ALT-801; right panel: 0.06 mg/kg ALT-801).

### DETAILED DESCRIPTION OF THE INVENTION

The present invention provides methods of treating cancer or symptoms thereof which comprise administering a therapeutically effective amount of a pharmaceutical composition comprising an IL-2 fusion protein and one or more therapeutic agents to a subject (e.g., a mammal such as a human). Thus, one embodiment is a method of treating a subject suffering from or susceptible to cancer or symptom thereof. The method includes the step of administering to the mammal a therapeutic amount of an IL-2 fusion protein and one or more therapeutic agents sufficient to treat the cancer or symptom thereof, under conditions such that the cancer is treated. The present invention also provides methods of treating cancer or symptoms thereof which comprise administering a therapeutically effective amount of an IL-2 fusion protein alone to a subject (e.g., a mammal such as a human).

The invention is based, at least in part, on the discovery that administration of IL-2 fusion protein in combination with one or more therapeutic agents to subjects having bladder cancer (also referred to herein as urothelial cancer) 1) ameliorated the cancer, 2) reduced tumor burden, 3) increased the survival of the subject, and 4) induced a durable immunological memory response against the cancer. In addition, IL-2 fusion protein combined with one or more therapeutic agents was found to be effective in treating chemo-resistant bladder cancers. Furthermore, IL-2 fusion proteins that do not specifically target the cancer cells or tissues were found to be as effective in treating bladder cancer as IL-2 fusion proteins that specifically target the cancer cells. In certain embodiments, IL-2 fusion protein monotherapy was found to be effective in treating bladder cancers, including chemo-resistant cancers.

It is well established that immunotherapy, including IL-2, is an effective approach for enhancing anti-tumor immunity against certain types of cancer. IL-2 has stimulatory effects on a number of immune cell types including T and B cells, monocytes, macrophages, lymphokine-activated killer cells (LAK) and NK cells (Waldmann, T. A., Nat Rev Immunol, 6: 595-601, 2006). Based on its ability to provide durable curative antitumor responses, systemic administration of recombinant human IL-2 (Proleukin®) has been approved to treat patients with metastatic melanoma or renal cell carcinoma (Rosenberg, S. A. et al., Ann Surg, 210: 474-484; discussion 484-475, 1989; Fyfe, G. et al., J Clin Oncol, 13: 688-696, 1995; and Atkins, M. B. et al., J Clin Oncol, 17: 2105-2116, 1999). Unfortunately, the considerable toxicity associated with this treatment makes it difficult to achieve an effective dose at the site of the tumor and limits the population that can be treated. For example, systemic treatment with IL-2 at tolerated doses induces lymphoid activation in virtually all treated patients, but anti-tumor responses are observed in a minority of these individuals (Rosenberg, S. A. et al., Ann Surg, 210: 474-484; discussion 484-475, 1989). As a result, use of high dose IL-2 is limited to specialized programs with experienced personnel and it is generally offered to patients who are responsive and have excellent organ function (Tarhini, A. A. et al., Curr Opin Investig Drugs, 6: 1234-1239, 2005). Lower dose IL-2 treatment, while less toxic and more convenient, produces lower response rates and appears to be ineffective in treating metastatic tumors (Yang, J. C. et al., J Clin Oncol, 21: 3127-3132, 2003). Local treatment (intravesical) of superficial bladder cancer patient with IL-2 has been shown to provide tumor regression and prolonged regression free time in a number of clinical studies (Den Otter, W. et al., J Urol, 159: 1183-1186, 1998; and Den Otter, W. et al., Cancer Immunol Immunother, 57: 931-950, 2008). In a Phase 2 study, systemic IL-2 administration to patients with cisplatin-refractory advance/metastatic urothelial carcinoma (65% of which were bladder cancers) provided a median survival of over 10 months compared to 6-7 months observed using single agent or combination salvage chemotherapy, suggesting further evidence of bladder carcinoma sensitivity to IL-2 therapy (Kim, J. et al., Urol Oncol, 21: 21-26, 2003; and Gallagher, D. J. et al., Cancer, 113: 1284-1293, 2008). However, IL-2 induced toxicities in these patients were significant and limited the treatment regimen (Kim, J. et al., Urol Oncol, 21: 21-26, 2003). Thus, there is a critical need for innovative strategies that enhance the curative effects of IL-2, reduce its toxicity without compromising clinical benefit and expand its utility beyond the currently approved indications.

Therapeutic strategies to specifically target malignancies have also been shown to be effective. However, although molecular and genetic markers for bladder cancer have been well characterized, there have been few clinical trials using molecular targeted agents against bladder cancer. Recent clinical studies in patients with advanced/metastatic bladder cancer using therapeutic antibodies (Abs) against HER-2/neu or VEGF or an oral EGFR antagonists have not shown improved efficacy/toxicity profiles compared to standard chemotherapy (Vaughn, D. J., J Clin Oncol, 25: 2162-2163, 2007; Hussain, M. H. et al., J Clin Oncol, 25: 2218-2224, 2007; 1 Hahn, N. M. et al., J Clin Oncol, 27: 5018, 2009; and Philips, G. K. et al., BJU Int, 101: 20-25, 2008), indicating that these targets are not appropriate for bladder cancer. Interestingly, genetic studies indicate that the pathogenesis of bladder cancer tumors mainly consists of two divergent, but overlapping pathways (Wu, X. R., Nat Rev Cancer, 5: 713-725, 2005). The non-muscle invasive bladder tumors are thought to arise from simple and nodular hyperplasia, and harbor frequent mutations in the fibroblast growth factor receptor 3, Ha-Ras, and PIK3CA genes. Muscle-invasive bladder cancer tumors are thought to originate from flat carcinoma in situ, severe dysphasia, or de novo. At least 50% of these tumors contain defects in the tumor suppressor p53 and/or retinoblastoma genes (Rosser, C. J. et al., Expert Rev Anticancer Ther, 1: 531-539, 2001). Consistent with this finding, elevated tumor overexpression of p53 correlates with progression of metastatic disease in bladder cancer patients (van Rhijn, B. W. G. et al., Cancer Research, 64: 1911-1914, 2004). This is also supported by transgenic mouse models of bladder cancer. Mice expressing SV40 Large T antigen (which binds to and inactivates the p53 protein) in urothelium develop carcinoma in situ and stochastic muscle-invasive carcinoma, whereas mice overexpressing Ha-ras develop hyperplasia and superficial disease (Zhang, Z. T. et al., Oncogene, 20: 1973-1980, 2001; and Zhang, Z. T. et al., Cancer Res, 59: 3512-3517, 1999).

Applicants identified the p53 protein in tumor cells as a target for therapeutic intervention. The very high-frequency occurrence of missense mutations in the p53 gene and subsequent overexpression of p53 protein in tumor cells creates the opportunity to target p53 as a tumor antigen in patients with advanced or metastatic bladder carcinoma. p53 is an intracellular tumor suppressor protein that acts to arrest the proliferation of cells (Levine, A. J. et al., Nature, 351: 453-456, 1991; and Vousden, K. H. and Prives, C., Cell, 120: 7-10, 2005). When mutated, it loses its ability to suppress abnormal proliferation and accumulates in tumor cells (Levine, A. J. et al., Nature, 351: 453-456, 1991; and Vousden, K. H. and Prives, C., Cell, 120: 7-10, 2005). As a result, p53 mutation/overexpression correlates with tumor aggression and recurrence and is associated with lower overall survival rates and resistance to chemotherapeutic intervention in a variety of cancer types including bladder cancer (van Rhijn, B. W. G. et al., Cancer Research, 64: 1911-1914, 2004; Strano, S. et al., Oncogene, 26: 2212-2219, 2007; and Goebell, P. J. et al., Urol Oncol, 28: 377-388, 2010). Recent analysis of over 3,400 bladder cancer patients revealed highly significant correlation between detectable p53 overexpression in tumor specimens versus tumor grade and tumor stage (Goebell, P. J. et al., Urol Oncol, 28: 377-388, 2010). Overexpression of p53 in tumors was also significantly correlated with tumor progression and poor survival of advanced bladder cancer patients. Since only low amounts of native p53 are detectable in normal tissue, the differential display of p53 in tumor versus normal tissues creates the opportunity to therapeutically target this protein. However, p53 is an intracellular protein and is not displayed on the cell surface, thus is not accessible to Ab-based agents. As with other intracellular proteins, p53 is processed and p53 peptides are presented at the cell surface in the context of HLA molecules. The Applicants identified a peptide epitope (aa264-272) of p53 presented by HLA-A*0201 that is displayed at high levels on the surface of different human tumor cells and tissues, whereas normal tissues do not present detectable levels of this complex. Since this epitope is within a region of p53 that is rarely mutated, its cell surface display serves as a broad-based target for tumors that overexpress p53. Applicants claimed method is based in part on the display of the p53 peptide epitope on the surface of human tumor cells.

As used herein, the terms “treat,” treating,” “treatment,” “therapy” and the like refer to reducing or ameliorating a disorder and/or symptoms associated therewith. It will be appreciated that, although not precluded, treating a disorder or condition does not require that the disorder, condition or symptoms associated therewith be completely eliminated.

As used herein, the terms “prevent,” “preventing,” “prevention,” “prophylactic treatment” and the like refer to reducing the probability of developing a disorder or condition in a subject, who does not have, but is at risk of or susceptible to developing a disorder or condition.

As used herein, the terms “effective,” “efficacy,” “efficacious” and the like refer to the ability to treat, prevent or ameliorate a disease, disorder and/or symptoms associated therewith.

The therapeutic methods of the invention (which include prophylactic treatment) in general comprise administration of a therapeutically effective amount of an IL-2 fusion protein in combination with one or more therapeutic agents to a subject (e.g., animal, human) in need thereof, including a mammal, particularly a human. Such treatment will be suitably administered to subjects, particularly humans, suffering from, having, susceptible to, or at risk for cancer, particularly bladder (or urothelial) cancer. Determination of those subjects “at risk” can be made by any objective or subjective determination by a diagnostic test or opinion of a subject or health care provider (e.g., genetic test, enzyme or protein marker, Marker (as defined herein), family history, and the like).

In one embodiment, the invention provides a method of monitoring treatment progress. The method includes the step of determining a level of diagnostic marker (Marker) (e.g., a protein or indicator thereof, etc.) or diagnostic measurement (e.g., screen, assay, scan for tumor size assessment, histopathological assessment in surgically removed tissue/biopsy, etc.) in a subject suffering from or susceptible to a disorder or symptoms thereof associated with cancer, particularly bladder cancer, in which the subject has been administered a therapeutic amount of a compound herein sufficient to treat the disease or symptoms thereof. The level of Marker or measurement determined in the method can be compared to known levels of Marker or measurement in either healthy normal controls or in other afflicted patients to establish the subject's disease status. In preferred embodiments, a second level of Marker or measurement in the subject is determined at a time point later than the determination of the first level, and the two levels are compared to monitor the course of disease or the efficacy of the therapy. In certain preferred embodiments, a pre-treatment level of Marker or measurement in the subject is determined prior to beginning treatment according to this invention; this pre-treatment level of Marker or measurement can then be compared to the level of Marker or measurement in the subject after the treatment commences, to determine the efficacy of the treatment. In certain preferred embodiments, monitoring of treatment efficacy is done based on the objective response of the cancer as assessed using the new international criteria proposed by the Response Evaluation Criteria in Solid Tumors Committee (RECIST) 1.1. In other embodiments, treatment efficacy is assessed based on subject overall survival or progression-free survival times or rates.

### Pharmaceutical Compositions

The methods described herein rely upon the administration of an IL-2 fusion protein alone or along with one or more therapeutic agents. The IL-2 fusion proteins of the invention comprise either the entire mature IL-2 polypeptide or a biologically active fragment thereof fused to a second polypeptide. In certain embodiments the second polypeptide has a targeting function in that it specifically binds to an epitope, peptide, ligand, or feature on a cancer cell. Accordingly, non-limiting examples of targeting polypeptides include antibodies and antigen binding fragments thereof, T cell receptors and peptide binding fragments thereof, and receptors and ligand binding fragments thereof. Any polypeptide that is able to specifically bind cancer cells may serve as the second polypeptide in a targeted IL-2 fusion protein.

Surprisingly, the invention provides that non-targeting IL-2 fusion proteins are as effective as targeted IL-2 fusion proteins in the described methods. The second polypeptide of a non-targeting IL-2 fusion protein includes antibodies and antigen binding fragments thereof, T cell receptors and peptide binding fragments thereof, and receptors and ligand binding fragments thereof. However, in these cases the second polypeptide does not specifically bind to the cancer cells to be treated. In preferred embodiments, the second polypeptide is a T cell receptor (TCR) and most preferably a single chain T cell receptor (scTCR). Examples of TCR molecules suitable for second polypeptides are described in U.S. Pat. Nos. 7,456,263; 6,534,633; U.S. Patent Application Publication No. US2003/0144474; and U.S. Patent Application Publication No. US2011/0070191, which are incorporated by reference herein in their entirety.

In particular, TCR fusion and conjugate complexes have been generated that have significantly increased utility as therapeutic molecules. Specifically, the new class of fusion molecules has been created that has increased cell surface residency time, and improved pharmacokinetic profiles, e.g., these molecules have a longer plasma half-life. The invention also provides expression vectors that encode such complexes that comprise a TCR molecule covalently linked to a biologically active polypeptide or molecule, and methods for production and use of such fusion and conjugate complexes and expression vectors and conjugate complexes.

A T cell recognizes antigen presented on the surfaces of cells by means of the T cell receptors expressed on their cell surface. TCRs are disulfide linked heterodimers, most consisting of α and β chain glycoproteins. T cells use mechanisms to generate diversity in their receptor molecules similar to those mechanisms for generating antibody diversity operating in B cells (Janeway and Travers; Immunobiology 1997). Similar to the immunoglobulin genes, TCR genes are composed of segments that rearrange during development of T cells. TCR polypeptides consist of amino terminal variable and carboxy terminal constant regions. While the carboxy terminal region functions as a transmembrane anchor and participates in intracellular signaling when the receptor is occupied, the variable region is responsible for recognition of antigens. The TCR α chain contains variable regions encoded by V and D segments only, while the β chain contains additional joining (J) segments. The rearrangement of these segments and the mutation and maturation of the variable regions results in a diverse repertoire of TCRs capable of recognizing an incredibly large number of different antigens displayed in the context of different TCR molecules.

Technology has been developed previously to produce highly specific T cell receptors (TCR) which recognize particular antigen. For example, the pending U. S. patent application U.S. Ser. No. 08/813,781 and U.S. Pat. No. 6,534,633, incorporated herein by reference in their entirety; and International publications PCT/US98/04274 and PCT/US99/24645, and references discussed therein disclose methods of preparing and using specific TCRs. Additionally, particular specific TCRs have been produced by recombinant methods as soluble, single-chain TCRs (scTCR). Methods for production and use of scTCRs have been disclosed and are described in International application PCT/US98/20263 which are incorporated herein by reference. Such TCRs and scTCRs can be altered so as to create fusions or conjugates to render the resulting TCRs and scTCRs useful as therapeutics. The TCR complexes of the invention can be generated by genetically fusing the recombinantly produced TCR or scTCR coding region to genes encoding biologically active polypeptide or molecules to produce TCR fusion complexes. Alternatively, a TCR or scTCRs can also be chemically conjugated with biologically active molecules to produce TCR conjugate complexes.

By the term “fusion molecule” as it is used herein is meant an IL-2 and second polypeptide, such as a TCR domain, covalently linked (i.e. fused) by recombinant, chemical or other suitable method. If desired, the fusion molecule can be fused at one or several sites through a peptide linker sequence. Alternatively, the peptide linker may be used to assist in construction of the fusion molecule. The fusion molecules of the invention exhibit improved characteristics that make them better therapeutic molecules.

The term “increased cell surface residency time” as used herein is meant to indicate that the claimed fusion molecules associate with proteins on the surface of cell for a longer period of time than any component of the fusion molecule does alone. In certain embodiments, the cell surface residency time is increased by 20%, 30%, 40%, 50%, 60%, 70%, 80%, 90%, 100% or more.

The term “serum half-life” or “plasma half-life” as used herein is intended to indicate the amount of time that is required for the concentration or amount of fusion molecule of the invention when in the body to be reduced to exactly one-half of a given concentration or amount. The fusion molecules of the invention display significantly longer half lives than IL-2 when not in a fusion molecule. For example, the serum half-life of the disclosed molecules can increase by 20%, 30%, 40%, 50%, 60%, 70%, 80%, 90%, 100%, 200%, 300%, 400%, 500%, 750%, 1000%, 1250%, 1500%, 1750%, 2000% or more over the serum half-life of the components of the claimed molecules when not part of a fusion protein.

A “polypeptide” refers to any polymer preferably consisting essentially of any of the 20 natural amino acids regardless of its size. Although the term “protein” is often used in reference to relatively large proteins, and “peptide” is often used in reference to small polypeptides, use of these terms in the field often overlaps. The term “polypeptide” refers generally to proteins, polypeptides, and peptides unless otherwise noted. Peptides useful in accordance with the present invention in general will be generally between about 0.1 to 100 KD or greater up to about 1000 KD, preferably between about 0.1, 0.2, 0.5, 1, 2, 5, 10, 20, 30 and 50 KD as judged by standard molecule sizing techniques such as centrifugation or SDS-polyacrylamide gel electrophoresis.

Additionally, the IL-2 fusion protein can be a detectably-labeled molecule suitable for diagnostic or imaging studies such as a fluorescent label such as green fluorescent protein, phycoerythrin, cychome, or Texas red; or a radionuclide e.g., iodine-131, yttrium-90, rhenium-188 or bismuth-212. See e.g., Moskaug, et al. J. Biol. Chem. 264, 15709 (1989); Pastan, I. et al. Cell 47, 641, 1986; Pastan et al., Recombinant Toxins as Novel Therapeutic Agents, Ann. Rev. Biochem. 61, 331, (1992); “Chimeric Toxins” Olsnes and Phil, Pharmac. Ther., 25, 355 (1982); published PCT application no. WO 94/29350; published PCT application no. WO 94/04689; and U.S. Pat. No. 5,620,939 for disclosure relating to making and using proteins comprising effectors or tags.

A specific example of an IL-2 fusion protein is as follows: an sc-TCR such as the c264sc-TCR fused to IL-2 (ALT-801) can be produced by transfecting mammalian cells. The c264scTCR/IL-2 protein fusion complex recognizes a processed peptide fragment from human wild-type p53 tumor suppressor protein presented in the context of human HLA antigen; HLA-2.1. The c264scTCR and its peptide ligand have been described in Card et al., Cancer Immunol Immunother (2004) 53: 345, Belmont, et al. Clin Immunol. (2006) 121:29, and Wen, et al. Cancer Immunol Immunother. (2008) 57:1781. The human p53 (aa264-aa272) peptide sequence (referred to herein as 264 peptide or p264) recognized by c264scTCR is LLGRNSFEV. Expression of tumor suppressor protein p53, is upregulated on malignant cells. In certain embodiments of the invention, recognition of tumor cells presenting p53 (aa264-aa272) peptide/HLA-A2 complexes on their surface by the c264scTCR/IL-2 protein fusion promotes immune activity against the tumor cells, hereby providing anti-cancer therapeutic activity. This targeted recognition can be beneficial in treated subjects with tumors that overexpress p53, including bladder tumors.

Other fusion molecules of the invention comprise IL-2 fused to other scTCRs specific for tumor associated or viral peptide antigens including those derived from MART-1, gp100, MAGE, HIV, Hepatitis A, B or C, CMV, AAV, LCMV, JCV, Influenza, HTLV and other viruses, wherein the scTCR is linked to an IL-2, either directly or through a linker. In addition, the IL-2 fusion proteins may further comprise additional polypeptide tags.

For example, one tag is a polypeptide bearing a charge at physiological pH, such as, e.g., 6×HIS. In this instance, the TCR fusion or conjugate complex can be purified by a commercially available metallo-sepharose matrix such as Ni-sepharose which is capable of specifically binding the 6×HIS tag at about pH 6-9. The EE epitope and myc epitope are further examples of suitable protein tags, which epitopes can be specifically bound by one or more commercially available monoclonal antibodies.

As noted, components of the fusion proteins disclosed herein, e.g., IL-2 and the second polypeptide, can be organized in nearly any fashion provided that the IL-2 fusion protein has the function for which it was intended. In particular, each component of the fusion protein can be spaced from another component by at least one suitable peptide linker sequence if desired. Furthermore, the components may be positioned by linkers such that IL-2 can bind its receptor and provide optimal immunostimulatory activity and/or the second polypeptide can bind its receptor/ligand and mediate its activity. Additionally, the fusion proteins may include tags, e.g., to facilitate identification and/or purification of the fusion protein.

The IL-2 fusion proteins of the invention have the surprising ability to increase either the plasma half-life of IL-2 (above the plasma half-life of IL-2 alone) or the surface residency time for the fusion molecules (above the surface residency time of IL-2 alone) that bind to a cell surface protein, e.g., a cell surface receptor. The IL-2 fusion proteins of the invention may have the ability to increase the plasma half-life of the molecule and increase the surface residency time of the molecule, thereby leading to significant increases in efficacy for the claimed molecules.

In general, preparation of the IL-2 fusion proteins of the invention can be accomplished by procedures disclosed herein and by recognized recombinant DNA techniques involving, e.g., polymerase chain amplification reactions (PCR), preparation of plasmid DNA, cleavage of DNA with restriction enzymes, preparation of oligonucleotides, ligation of DNA, isolation of mRNA, introduction of the DNA into a suitable cell, transformation or transfection of a host, culturing of the host. Additionally, the fusion molecules can be isolated and purified using chaotropic agents and well known electrophoretic, centrifugation and chromatographic methods. See generally, Sambrook et al., Molecular Cloning: A Laboratory Manual (2nd ed. (1989); and Ausubel et al., Current Protocols in Molecular Biology, John Wiley & Sons, New York (1989) for disclosure relating to these methods.

The invention further provides nucleic acid sequences and particularly DNA sequences that encode the present fusion proteins. Preferably, the DNA sequence is carried by a vector suited for extrachromosomal replication such as a phage, virus, plasmid, phagemid, cosmid, YAC, or episome. In particular, a DNA vector that encodes a desired fusion protein can be used to facilitate preparative methods described herein and to obtain significant quantities of the fusion protein. The DNA sequence can be inserted into an appropriate expression vector, i.e., a vector which contains the necessary elements for the transcription and translation of the inserted protein-coding sequence. A variety of host-vector systems may be utilized to express the protein-coding sequence. These include mammalian cell systems infected with virus (e.g., vaccinia virus, adenovirus, etc.); insect cell systems infected with virus (e.g., baculovirus); microorganisms such as yeast containing yeast vectors, or bacteria transformed with bacteriophage DNA, plasmid DNA or cosmid DNA. Depending on the host-vector system utilized, any one of a number of suitable transcription and translation elements may be used. See generally Sambrook et al., supra and Ausubel et al. supra.

In general, a preferred DNA vector according to the invention comprises a nucleotide sequence linked by phosphodiester bonds comprising, in a 5′ to 3′ direction a first cloning site for introduction of a first nucleotide sequence encoding a TCR chain, operatively linked to a sequence encoding IL-2.

In most instances, it will be preferred that each of the fusion protein components encoded by the DNA vector be provided in a “cassette” format. By the term “cassette” is meant that each component can be readily substituted for another component by standard recombinant methods.

To make the vector coding for a TCR fusion complex, the sequence coding for the TCR molecule is linked to a sequence coding for IL-2 by use of suitable ligases. DNA coding for the presenting peptide can be obtained by isolating DNA from natural sources such as from a suitable cell line or by known synthetic methods, e.g. the phosphate triester method. See, e.g., Oligonucleotide Synthesis, IRL Press (M. J. Gait, ed., 1984). Synthetic oligonucleotides also may be prepared using commercially available automated oligonucleotide synthesizers. Once isolated, the gene coding for the TCR molecule can be amplified by the polymerase chain reaction (PCR) or other means known in the art. Suitable PCR primers to amplify the TCR peptide gene may add restriction sites to the PCR product. The PCR product preferably includes splice sites for the IL-2 polypeptide and leader sequences necessary for proper expression and secretion of the TCR-IL-2 fusion complex. The PCR product also preferably includes a sequence coding for the linker sequence, or a restriction enzyme site for ligation of such a sequence.

The fusion proteins described herein are preferably produced by standard recombinant DNA techniques. For example, once a DNA molecule encoding the TCR protein is isolated, sequence can be ligated to another DNA molecule encoding the IL-2 polypeptide. The nucleotide sequence coding for a TCR molecule may be directly joined to a DNA sequence coding for the IL-2 peptide or, more typically, a DNA sequence coding for the linker sequence as discussed herein may be interposed between the sequence coding for the TCR molecule and the sequence coding for the IL-2 peptide and joined using suitable ligases. The resultant hybrid DNA molecule can be expressed in a suitable host cell to produce the IL-2 fusion protein. The DNA molecules are ligated to each other in a 5′ to 3′ orientation such that, after ligation, the translational frame of the encoded polypeptides is not altered (i.e., the DNA molecules are ligated to each other in-frame). The resulting DNA molecules encode an in-frame fusion protein.

Other nucleotide sequences also can be included in the gene construct. For example, a promoter sequence, which controls expression of the sequence coding for the TCR peptide fused to the IL-2 peptide, or a leader sequence, which directs the IL-2 fusion protein to the cell surface or the culture medium, can be included in the construct or present in the expression vector into which the construct is inserted. An immunoglobulin or CMV promoter is particularly preferred.

The components of the fusion protein can be organized in nearly any order provided each is capable of performing its intended function. For example, in one embodiment, the TCR is situated at the C or N terminal end of the IL-2 molecule.

As noted, a fusion molecule or a conjugate molecule in accord with the invention can be organized in several ways. In an exemplary configuration, the C-terminus of the TCR is operatively linked to the N-terminus of the IL-2 molecule. That linkage can be achieved by recombinant methods if desired. However, in another configuration, the N-terminus of the TCR is linked to the C-terminus of the IL-2 molecule.

Preferably the linker sequence comprises from about 1 to 20 amino acids, more preferably from about 1 to 16 amino acids. The linker sequence is preferably flexible so as not hold the IL-2 in a single undesired conformation. The linker sequence can be used, e.g., to space the recognition site from the fused molecule. Specifically, the peptide linker sequence can be positioned between the TCR chain and the IL-2 peptide, e.g., to chemically cross-link same and to provide molecular flexibility. The linker is preferably predominantly comprises amino acids with small side chains, such as glycine, alanine and serine, to provide for flexibility. Preferably about 80 or 90 percent or greater of the linker sequence comprises glycine, alanine or serine residues, particularly glycine and serine residues. For an IL-2 fusion protein that contains a heterodimer TCR, the linker sequence is suitably linked to the β chain of the TCR molecule, although the linker sequence also could be attached to the α chain of the TCR molecule. Alternatively, linker sequence may be linked to both α and β chains of the TCR molecule to create a single-chain molecule. Suitable linker sequences are SGGGGSGGG (i.e., Ser Gly Gly Gly Gly Ser Gly Gly Gly), TSGGGGSGGGGSGGGGSGGGGSS and VNAKTTAPSVYPLAPVSQ. Different linker sequences could be used including any of a number of flexible linker designs that have been used successfully to join antibody variable regions together, see Whitlow, M. et al., (1991) Methods: A Companion to Methods in Enzymology 2:97-105. Suitable linker sequences can be readily identified empirically. Additionally, suitable size and sequences of linker sequences also can be determined by conventional computer modeling techniques based on the predicted size and shape of the TCR molecule.

A number of strategies can be employed to express IL-2 fusion proteins of the invention. For example, the IL-2 gene fusion construct described above can be incorporated into a suitable vector by known means such as by use of restriction enzymes to make cuts in the vector for insertion of the construct followed by ligation. The vector containing the gene construct is then introduced into a suitable host for expression of the IL-2 fusion peptide. See, generally, Sambrook et al., supra. Selection of suitable vectors can be made empirically based on factors relating to the cloning protocol. For example, the vector should be compatible with, and have the proper replicon for the host that is being employed. Further the vector must be able to accommodate the DNA sequence coding for the IL-2 fusion protein that is to be expressed. Suitable host cells include eukaryotic and prokaryotic cells, preferably those cells that can be easily transformed and exhibit rapid growth in culture medium. Specifically preferred hosts cells include prokaryotes such as E. coli, Bacillus subtillus, etc. and eukaryotes such as animal cells and yeast strains, e.g., S. cerevisiae. Mammalian cells are generally preferred, particularly J558, NSO, SP2-O or CHO. Other suitable hosts include, e.g., insect cells such as Sf9. Conventional culturing conditions are employed. See Sambrook, supra. Stable transformed or transfected cell lines can then be selected. Cells expressing a TCR fusion complex of the invention can be determined by known procedures. For example, expression of a TCR fusion complex linked to an immunoglobulin can be determined by an ELISA specific for the linked immunoglobulin and/or by immunoblotting.

As mentioned generally above, a host cell can be used for preparative purposes to propagate nucleic acid encoding a desired fusion protein. Thus a host cell can include a prokaryotic or eukaryotic cell in which production of the fusion protein is specifically intended. Thus host cells specifically include yeast, fly, worm, plant, frog, mammalian cells and organs that are capable of propagating nucleic acid encoding the fusion. Non-limiting examples of mammalian cell lines which can be used include CHO dhfr− cells (Urlaub and Chasm, Proc. Natl. Acad. Sci. USA, 77:4216 (1980)), 293 cells (Graham et al., J Gen. Virol., 36:59 (1977)) or myeloma cells like SP2 or NSO (Galfre and Milstein, Meth. Enzymol., 73(B):3 (1981)).

Host cells capable of propagating nucleic acid encoding a desired fusion protein encompass non-mammalian eukaryotic cells as well, including insect (e.g., Sp. frugiperda), yeast (e.g., S. cerevisiae, S. pombe, P. pastoris., K lactis, H. polymorpha; as generally reviewed by Fleer, R., Current Opinion in Biotechnology, 3(5):486496 (1992)), fungal and plant cells. Also contemplated are certain prokaryotes such as E. coli and Bacillus.

Nucleic acid encoding a desired fusion protein can be introduced into a host cell by standard techniques for transfecting cells. The term “transfecting” or “transfection” is intended to encompass all conventional techniques for introducing nucleic acid into host cells, including calcium phosphate co-precipitation, DEAE-dextran-mediated transfection, lipofection, electroporation, microinjection, viral transduction and/or integration. Suitable methods for transfecting host cells can be found in Sambrook et al. supra, and other laboratory textbooks.

The present invention further provides a production process for isolating an IL-2 fusion protein of interest. In the process, a host cell (e.g., a yeast, fungus, insect, bacterial or animal cell), into which has been introduced a nucleic acid encoding the protein of the interest operatively linked to a regulatory sequence, is grown at production scale in a culture medium in the presence of the fusion protein to stimulate transcription of the nucleotides sequence encoding the fusion protein of interest. Subsequently, the fusion protein of interest is isolated from harvested host cells or from the culture medium. Standard protein purification techniques can be used to isolate the protein of interest from the medium or from the harvested cells. In particular, the purification techniques can be used to express and purify a desired fusion protein on a large-scale (i.e. in at least milligram quantities) from a variety of implementations including roller bottles, spinner flasks, tissue culture plates, bioreactor, or a fermentor.

An expressed IL-2 fusion protein can be isolated and purified by known methods. Typically the culture medium is centrifuged and then the supernatant is purified by affinity or immunoaffinity chromatography, e.g. Protein-A or Protein-G affinity chromatography or an immunoaffinity protocol comprising use of monoclonal antibodies that bind the expressed fusion complex such as a linked TCR or immunoglobulin region thereof. The fusion proteins of the present invention can be separated and purified by appropriate combination of known techniques. These methods include, for example, methods utilizing solubility such as salt precipitation and solvent precipitation, methods utilizing the difference in molecular weight such as dialysis, ultra-filtration, gel-filtration, and SDS-polyacrylamide gel electrophoresis, methods utilizing a difference in electrical charge such as ion-exchange column chromatography, methods utilizing specific affinity such as affinity chromatograph, methods utilizing a difference in hydrophobicity such as reverse-phase high performance liquid chromatograph and methods utilizing a difference in isoelectric point, such as isoelectric focusing electrophoresis, metal affinity columns such as Ni-NTA. See generally Sambrook et al. and Ausubel et al. supra for disclosure relating to these methods.

It is preferred that the IL-2 fusion proteins of the present invention be substantially pure. That is, the fusion proteins have been isolated from cell substituents that naturally accompany it so that the fusion proteins are present preferably in at least 80% or 90% to 95% homogeneity (w/w). Fusion proteins having at least 98 to 99% homogeneity (w/w) are most preferred for many pharmaceutical, clinical and research applications. Once substantially purified the fusion protein should be substantially free of contaminants for therapeutic applications. Once purified partially or to substantial purity, the soluble fusion proteins can be used therapeutically, or in performing in vitro or in vivo assays as disclosed herein. Substantial purity can be determined by a variety of standard techniques such as chromatography and gel electrophoresis.

Truncated IL-2 fusion proteins of the invention contain a TCR molecule that is sufficiently truncated so the TCR fusion complex can be secreted into culture medium after expression. Thus, a truncated IL-2 fusion protein will not include regions rich in hydrophobic residues, typically the transmembrane and cytoplasmic domains of the TCR molecule. Thus, for example, for a preferred truncated TCR molecule of the invention, preferably from about residues 199 to 237 of the β chain and from about residues 193 to 230 of the α chain of the TCR molecule are not included in the truncated TCR fusion complex.

The term “misfolded” as it relates to the fusion proteins is meant a protein that is partially or completely unfolded (i.e. denatured). A fusion protein can be partially or completely misfolded by contact with one or more chaotropic agents as discussed below. More generally, misfolded fusion proteins disclosed herein are representative of a high Gibbs free energy (ΔG) form of the corresponding native protein. Preferred are native fusion protein which is usually correctly folded, it is fully soluble in aqueous solution, and it has a relatively low ΔG. Accordingly, that native fusion protein is stable in most instances.

It is possible to detect fusion protein misfolding by one or a combination of conventional strategies. For example, the misfolding can be detected by a variety of conventional biophysical techniques including optical rotation measurements using native (control) and misfolded molecules.

By the term “soluble” or similar term is meant that the fusion molecule and particularly a fusion protein that is not readily sedimented under low G-force centrifugation (e.g. less than about 30,000 revolutions per minute in a standard centrifuge) from an aqueous buffer, e.g., cell media. Further, the fusion molecule is soluble if the it remains in aqueous solution at a temperature greater than about 5-37° C. and at or near neutral pH in the presence of low or no concentration of an anionic or non-ionic detergent. Under these conditions, a soluble protein will often have a low sedimentation value e.g., less than about 10 to 50 svedberg units.

Aqueous solutions referenced herein typically have a buffering compound to establish pH, typically within a pH range of about 5-9, and an ionic strength range between about 2 mM and 500 mM. Sometimes a protease inhibitor or mild non-ionic detergent is added. Additionally, a carrier protein may be added if desired such as bovine serum albumin (BSA) to a few mg/ml. Exemplary aqueous buffers include standard phosphate buffered saline, tris-buffered saline, or other well known buffers and cell media formulations.

### Pharmaceutical Therapeutics

The invention includes IL-2 fusion proteins that are useful for the treatment of neoplasia. In one particular embodiment, the IL-2 fusion proteins of the invention are useful for preventing or reducing tumor growth or for reducing the propensity of a neoplastic cell to invade a surrounding tissue or to otherwise metastasize. For therapeutic uses, the IL-2 fusion proteins disclosed herein may be administered systemically, for example, formulated in a pharmaceutically-acceptable buffer such as physiological saline. Preferable routes of administration include, for example, subcutaneous, intravenous, interperitoneally, intramuscular, or intradermal injections that provide continuous, sustained levels of the drug in the patient. Treatment of human patients or other animals will be carried out using a therapeutically effective amount of a therapeutic identified herein in a physiologically-acceptable carrier. Suitable carriers and their formulation are described, for example, in Remington's Pharmaceutical Sciences by E. W. Martin. The amount of the therapeutic agent to be administered varies depending upon the manner of administration, the age and body weight of the patient, and with the clinical symptoms of the neoplasia. Generally, amounts will be in the range of those used for other agents used in the treatment of other diseases associated with neoplasia, although in certain instances lower amounts will be needed because of the increased specificity of the compound.

### Therapeutic Methods

The IL-2 fusion proteins of the invention are useful for preventing or ameliorating neoplastic disease. In one therapeutic approach, an agent identified or described herein is administered to the site of a potential or actual disease-affected tissue or is administered systemically. The dosage of the administered agent depends on a number of factors, including the size and health of the individual patient. For any particular subject, the specific dosage regimes should be adjusted over time according to the individual need and the professional judgment of the person administering or supervising the administration of the compositions.

### Formulation of Pharmaceutical Compositions

The administration of a therapeutic agent for the treatment of neoplasia may be by any suitable means that results in a concentration of the therapeutic that, combined with other components, is effective in ameliorating, reducing, or stabilizing a neoplasia. The compound may be contained in any appropriate amount in any suitable carrier substance, and is generally present in an amount of 1-95% by weight of the total weight of the composition. The composition may be provided in a dosage form that is suitable for parenteral (e.g., subcutaneously, intravenously, intramuscularly, intravesicularly or intraperitoneally) administration route. An advantageous method of administration is intravenous infusion. The pharmaceutical therapeutic agent may be formulated according to conventional pharmaceutical practice (see, e.g., Remington: The Science and Practice of Pharmacy (20th ed.), ed. A. R. Gennaro, Lippincott Williams & Wilkins, 2000 and Encyclopedia of Pharmaceutical Technology, eds. J. Swarbrick and J. C. Boylan, 1988-1999, Marcel Dekker, New York).

Pharmaceutical compositions according to the invention may be formulated to release the IL-2 fusion protein substantially immediately upon administration or at any predetermined time or time period after administration. The latter types of compositions are generally known as controlled release formulations, which include (i) formulations that create a substantially constant concentration of the drug within the body over an extended period of time; (ii) formulations that after a predetermined lag time create a substantially constant concentration of the drug within the body over an extended period of time; (iii) formulations that sustain action during a predetermined time period by maintaining a relatively, constant, effective level in the body with concomitant minimization of undesirable side effects associated with fluctuations in the plasma level of the active substance (sawtooth kinetic pattern); (iv) formulations that localize action by, e.g., spatial placement of a controlled release composition adjacent to or in contact with the thymus; (v) formulations that allow for convenient dosing, such that doses are administered, for example, once every one or two weeks; and (vi) formulations that neoplasia by using carriers or chemical derivatives to deliver the therapeutic agent to a particular cell type (e.g., neoplastic cell). For some applications, controlled release formulations obviate the need for frequent dosing during the day to sustain the plasma level at a therapeutic level.

Any of a number of strategies can be pursued in order to obtain controlled release in which the rate of release outweighs the rate of metabolism of the compound in question. In one example, controlled release is obtained by appropriate selection of various formulation parameters and ingredients, including, e.g., various types of controlled release compositions and coatings. Thus, the therapeutic is formulated with appropriate excipients into a pharmaceutical composition that, upon administration, releases the therapeutic in a controlled manner. Examples include single or multiple unit tablet or capsule compositions, oil solutions, suspensions, emulsions, microcapsules, microspheres, molecular complexes, nanoparticles, patches, and liposomes.

### Parenteral Compositions

The pharmaceutical composition may be administered parenterally by injection, infusion or implantation (subcutaneous, intravenous, intramuscular, intraperitoneal, intravesicularly or the like) in dosage forms, formulations, or via suitable delivery devices or implants containing conventional, non-toxic pharmaceutically acceptable carriers and adjuvants. The formulation and preparation of such compositions are well known to those skilled in the art of pharmaceutical formulation. Formulations can be found in Remington: The Science and Practice of Pharmacy, supra.

Compositions for parenteral use may be provided in unit dosage fort is (e.g., in single-dose ampoules), or in vials containing several doses and in which a suitable preservative may be added (see below). The composition may be in the form of a solution, a suspension, an emulsion, an infusion device, or a delivery device for implantation, or it may be presented as a dry powder to be reconstituted with water or another suitable vehicle before use. Apart from the active agent that reduces or ameliorates a neoplasia, the composition may include suitable parenterally acceptable carriers and/or excipients. The active therapeutic agent(s) may be incorporated into microspheres, microcapsules, nanoparticles, liposomes, or the like for controlled release. Furthermore, the composition may include suspending, solubilizing, stabilizing, pH-adjusting agents, tonicity adjusting agents, and/or dispersing, agents.

As indicated above, the pharmaceutical compositions according to the invention may be in the form suitable for sterile injection. To prepare such a composition, the suitable therapeutic(s) are dissolved or suspended in a parenterally acceptable liquid vehicle. Among acceptable vehicles and solvents that may be employed are water, water adjusted to a suitable pH by addition of an appropriate amount of hydrochloric acid, sodium hydroxide or a suitable buffer, 1,3-butanediol, Ringer's solution, and isotonic sodium chloride solution and dextrose solution. The aqueous formulation may also contain one or more preservatives (e.g., methyl, ethyl or n-propyl p-hydroxybenzoate). In cases where one of the compounds is only sparingly or slightly soluble in water, a dissolution enhancing or solubilizing agent can be added, or the solvent may include 10-60% w/w of propylene glycol or the like.

### Controlled Release Parenteral Compositions

Controlled release parenteral compositions may be in form of aqueous suspensions, microspheres, microcapsules, magnetic microspheres, oil solutions, oil suspensions, or emulsions. Alternatively, the antibody may be incorporated in biocompatible carriers, liposomes, nanoparticles, implants, or infusion devices.

Materials for use in the preparation of microspheres and/or microcapsules are, e.g., biodegradable/bioerodible polymers such as polygalactin, poly-(isobutyl cyanoacrylate), poly(2-hydroxyethyl-L-glutam-nine) and, poly(lactic acid). Biocompatible carriers that may be used when formulating a controlled release parenteral formulation are carbohydrates (e.g., dextrans), proteins (e.g., albumin), lipoproteins, or antibodies. Materials for use in implants can be non-biodegradable (e.g., polydimethyl siloxane) or biodegradable (e.g., poly(caprolactone), poly(lactic acid), poly(glycolic acid) or poly(ortho esters) or combinations thereof).

### Solid Dosage Forms for Oral Use

Formulations for oral use include tablets containing the active ingredient(s) in a mixture with non-toxic pharmaceutically acceptable excipients. Such formulations are known to the skilled artisan. Excipients may be, for example, inert diluents or fillers (e.g., sucrose, sorbitol, sugar, mannitol, microcrystalline cellulose, starches including potato starch, calcium carbonate, sodium chloride, lactose, calcium phosphate, calcium sulfate, or sodium phosphate); granulating and disintegrating agents (e.g., cellulose derivatives including microcrystalline cellulose, starches including potato starch, croscarmellose sodium, alginates, or alginic acid); binding agents (e.g., sucrose, glucose, sorbitol, acacia, alginic acid, sodium alginate, gelatin, starch, pregelatinized starch, microcrystalline cellulose, magnesium aluminum silicate, carboxymethylcellulose sodium, methylcellulose, hydroxypropyl methylcellulose, ethylcellulose, polyvinylpyrrolidone, or polyethylene glycol); and lubricating agents, glidants, and antiadhesives (e.g., magnesium stearate, zinc stearate, stearic acid, silicas, hydrogenated vegetable oils, or talc). Other pharmaceutically acceptable excipients can be colorants, flavoring agents, plasticizers, humectants, buffering agents, and the like.

The tablets may be uncoated or they may be coated by known techniques, optionally to delay disintegration and absorption in the gastrointestinal tract and thereby providing a sustained action over a longer period. The coating may be adapted to release the active drug in a predetermined pattern (e.g., in order to achieve a controlled release formulation) or it may be adapted not to release the active drug until after passage of the stomach (enteric coating). The coating may be a sugar coating, a film coating (e.g., based on hydroxypropyl methylcellulose, methylcellulose, methyl hydroxyethylcellulose, hydroxypropylcellulose, carboxymethylcellulose, acrylate copolymers, polyethylene glycols and/or polyvinylpyrrolidone), or an enteric coating (e.g., based on methacrylic acid copolymer, cellulose acetate phthalate, hydroxypropyl methylcellulose phthalate, hydroxypropyl methylcellulose acetate succinate, polyvinyl acetate phthalate, shellac, and/or ethylcellulose). Furthermore, a time delay material, such as, e.g., glyceryl monostearate or glyceryl distearate may be employed.

The solid tablet compositions may include a coating adapted to protect the composition from unwanted chemical changes, (e.g., chemical degradation prior to the release of the chimeric antibody). The coating may be applied on the solid dosage form in a similar manner as that described in Encyclopedia of Pharmaceutical Technology, supra.

Formulations for oral use may also be presented as chewable tablets, or as hard gelatin capsules wherein the active ingredient is mixed with an inert solid diluent (e.g., potato starch, lactose, microcrystalline cellulose, calcium carbonate, calcium phosphate or kaolin), or as soft gelatin capsules wherein the active ingredient is mixed with water or an oil medium, for example, peanut oil, liquid paraffin, or olive oil. Powders and granulates may be prepared using the ingredients mentioned above under tablets and capsules in a conventional manner using, e.g., a mixer, a fluid bed apparatus or a spray drying equipment.

### Controlled Release Oral Dosage Forms

Controlled release compositions for oral use may, e.g., be constructed to release the chimeric antibody therapeutic by controlling the dissolution and/or the diffusion of the active substance. Dissolution or diffusion controlled release can be achieved by appropriate coating of a tablet, capsule, pellet, or granulate formulation of compounds, or by incorporating the compound into an appropriate matrix. A controlled release coating may include one or more of the coating substances mentioned above and/or, e.g., shellac, beeswax, glycowax, castor wax, carnauba wax, stearyl alcohol, glyceryl monostearate, glyceryl distearate, glycerol palmitostearate, ethylcellulose, acrylic resins, dl-polylactic acid, cellulose acetate butyrate, polyvinyl chloride, polyvinyl acetate, vinyl pyrrolidone, polyethylene, polymethacrylate, methylmethacrylate, 2-hydroxymethacrylate, methacrylate hydrogels, 1,3 butylene glycol, ethylene glycol methacrylate, and/or polyethylene glycols. In a controlled release matrix formulation, the matrix material may also include, e.g., hydrated methylcellulose, carnauba wax and stearyl alcohol, carbopol 934, silicone, glyceryl tristearate, methyl acrylate-methyl methacrylate, polyvinyl chloride, polyethylene, and/or halogenated fluorocarbon.

A controlled release composition containing one or more therapeutic compounds may also be in the form of a buoyant tablet or capsule (i.e., a tablet or capsule that, upon oral administration, floats on top of the gastric content for a certain period of time). A buoyant tablet formulation of the compound(s) can be prepared by granulating a mixture of the compound(s) with excipients and 20-75% w/w of hydrocolloids, such as hydroxyethylcellulose, hydroxypropylcellulose, or hydroxypropylmethylcellulose. The obtained granules can then be compressed into tablets. On contact with the gastric juice, the tablet forms a substantially water-impermeable gel barrier around its surface. This gel barrier takes part in maintaining a density of less than one, thereby allowing the tablet to remain buoyant in the gastric juice.

### Combination Therapy

The invention provides for the combined administration of an IL-2 fusion protein and one or more therapeutic agents. The IL-2 fusion protein may be administered before, concurrently, or after administration of the therapeutic agents. Moreover, if more than one therapeutic agent is used these agents may be administered concurrently or separately. In addition, the administration of the IL-2 fusion proteins and one or more therapeutic agents may be administered in various dosage schedules. In certain embodiments the IL-2 fusion protein and the one or more therapeutic agents are administered in multiple dosing schedules that may be separated by one or more rest periods.

The combination of an IL-2 fusion protein and one or more therapeutic agent of the invention in a neoadjuvant setting prior to additional therapy or surgery or as first line, second line or later line therapy depending on the disease stage of the patient. In preferred embodiments, the combined therapy is given to subjects with bladder cancer prior to cystectomy. Such therapy may eradicate micrometastases, downstage tumor, reduce implantation of circulating tumor cells post-surgery and improve survival. In other embodiments, the combined therapy of the invention is given to subjects with advanced or metastatic bladder cancer as a first line or second line therapy. Such treatment can be provided to subjects who are resistant or ineligible for standard therapies. Use of the IL-2 fusion protein as monotherapy may also be effectively used in these treatment settings.

The combination of an IL-2 fusion protein and one or more therapeutic agent of the invention may be advantageous in providing a more efficacious therapy than treatment with the individual agents. In certain preferred embodiments, the combined therapy comprises ALT-801 as the IL-2 fusion protein and cisplatin and/or gemcitabine as the therapeutic agents. Additionally, embodiments of the invention include treatment of subjects with bladder (or urothelial) cancer, wherein said cancer may be transitional cell carcinoma, carcinoma (or tumor) in situ, nonmuscle-invasive, muscle-invasive, locally advanced, metastatic, Stage I through IV, or low or high grade.

In preferred embodiments, combined administration of an IL-2 fusion protein and one or more therapeutic agents is more effective at treating or preventing cancer in subjects than treatment with the therapeutic agents alone. The effectiveness of the combined treatment using IL-2 fusion protein and one or more therapeutic agents can be compared to treatment with therapeutic agents alone on prospective or retrospective basis, using historic efficacy measures of similar study groups or in cross-over studies. Measurements of efficacy may are well established for cancer treatment and may include overall tumor responses (i.e. rates of progressive disease, stable disease, partial responses or complete responses based on RECIST, WHO or other criteria), progression free survival, time to progression, overall survival or survival rates, hazard ratios, relapse rate or time, tumor biomarker analysis, quality of life measurements, rate of or time to additional treatment, etc. Better efficacy of the combined treatment using IL-2 fusion protein and one or more therapeutic agents compared treatment with the therapeutic agents alone is typically defined as a statistically significant improvement (i.e. P value <0.10 or preferably <0.05) in the efficacy measure or may be defined as an increase in time to event measures of weeks, months or years or an improvement rate measures by 1%, 5%, 10%, 15%, 20%, 30%, 40%, 50%, 60%, 70%, 80%, 90%, 100%, 200%, 300%, 400%, 500%, 750%, 1000%, 1250%, 1500%, 1750%, 2000% or more. In a non-limiting example, treatment of subjects with advance or metastatic bladder cancer using the combined administration of ALT-801 and gemcitabine+cisplatin of the invention provided better anti-tumor efficacy than that previously reported for advance/metastatic bladder cancer subjects treated with gemcitabine+cisplatin or other cisplatin-based chemotherapy regimens. Specifically, von der Maase et al. (J. Clin. Oncol. (2000) 17:3068) reported in a Phase III clinical study of patients with advanced or metastatic bladder cancer, treatment with gemcitabine+cisplatin resulted in an overall tumor response rate (i.e., rate of partial response and complete response) of 49.4% (81 of 182 assessed patients) and a complete response rate of 12.2% by independent radiologic review. This study also reported a similar overall response rate (45.7%, 69 of 181 assessed patients) and complete response rate (11.9%) in patients treated with methotrexate, vinblastine, doxorubicin, and cisplatin. Subsequent studies of other chemotherapy regimens (i.e., single agents, doublets, triplets) in this patient population reported similar or inferior response rates (reviewed by Yafi et al. Curr. Oncol. (2011) 18:e25). Surprisingly, combined administration of ALT-801 and gemcitabine+cisplatin of the invention to patients with advance/metastatic bladder (urothelial) cancer provided much better overall response and complete response rates than that reported for gemcitabine+cisplatin or other cisplatin-based chemotherapy regimens by von der Maase et al. or others.

Additionally, combined administration of an IL-2 fusion protein and one or more therapeutic agents is effective at treating or preventing cancer in subjects that are resistant to chemotherapy. In certain embodiments, combined treatment of the inventions includes one or more therapeutic agents for which the cancer is resistant. In other embodiments, combined treatment of the inventions includes one or more therapeutic agents which are different from that which the cancer is resistant. In a non-limiting example, the combined administration of ALT-801 and gemcitabine+cisplatin was efficacious in providing complete response (CR) in patients with bladder cancer that progressed on previous gemcitabine+cisplatin therapy. This result is high unexpected given fact that no CRs were reported in a Phase III study of 370 patients with advanced urothelial cancer who progressed after a platinum-containing regimen (Bellmunt et al. J. Clin. Oncol. (2009) 27: 4454).

The combination of an IL-2 fusion protein and one or more therapeutic agent of the invention may provide more efficacious therapy through a variety of mechanisms. The IL-2 fusion protein and cytotoxic therapeutic agent regimen can provide efficacy through the combination of direct effects of these agents on the cancer. In some circumstances, the timing of these effects may provide improved outcomes. For example, rapid activity of a cytotoxic therapeutic agent against bulky disease in combination with durable long-term activity of an IL-2 fusion protein against residual disease could provide better efficacy than either agent alone. Alternatively, therapeutic agents not only have direct cytotoxic effects on tumor cells but may also potentiate the immune system via so-called off-target effects to achieve efficient anti-cancer immunity in combination with the IL-2 fusion protein of the invention (Galluzzi, L. et al., Nat Rev Drug Discov, 11: 215-233). For example, treatment with the therapeutic agent may increase the expression of an antigenic target on the cancer cell surface, thereby allowing more effective anti-tumor immune responses induced by the IL-2 fusion protein. In some embodiments, the antigenic target is recognized by a component of the IL-2 fusion protein and immune responses are directed against the tumor cells via IL-2 fusion protein interaction. In one example, the therapeutic agent increases the HLA or HLA/peptide complex levels on the tumor cell surface and enhances recognition by a TCR-IL2 fusion protein. In other specific examples, platinum-based compounds, including cisplatin, oxaliplatin and carboplatin, not only induce class I HLA expression but markedly reduce the expression of the T cell inhibitory molecule PD-L2 on human tumor cells (Lesterhuis, W. J. et al., J Clin Invest, 121: 3100-3108). Down-regulation of PD-L2 could result in enhanced anti-tumor effects of T cells stimulated by an IL-2 fusion protein. Various therapeutic agents, including cisplatin, paclitaxel and doxorubicin, have the capability to sensitize tumor cells to cytotoxic T lymphocytes (CTLs) by increasing the permeability of tumor cells to granzyme, thereby rendering them susceptible to CTL-mediated lysis even if they do not express the antigen recognized by CTLs (Ramakrishnan, R. et al., J Clin Invest, 120: 1111-1124). In other embodiments of the invention, the combination of an IL-2 fusion protein with gemcitabine can result in more efficacious therapy due to the activity of gemcitabine to increase the expression of class I HLA on tumor cells and to enhance the cross-presentation of tumor antigen to the CD8⁺ T cells activated by the IL-2 fusion protein (Liu, W. M. et al., Br J Cancer, 102: 115-123; Nowak, A. K. et al., J Immunol, 170: 4905-4913, 2003; and Nowak, A. K. et al., Cancer Res, 63: 4490-4496, 2003). In the combination therapy of the invention, use of gemcitabine may also selectively kill myeloid-derived suppressor cells (MDSCs) responsible for suppressing antigen-specific T-cell responses (Mundy-Bosse, B. L. et al., Cancer Res, 71: 5101-5110; Vincent, J. et al., Cancer Res, 70: 3052-3061; Suzuki, E. et al., Clin Cancer Res, 11: 6713-6721, 2005; and Ko, H. J. et al., Cancer Res, 67: 7477-7486, 2007), thereby providing a better environment for IL-2 fusion protein-mediated anti-tumor immune activity. Chemotherapy may also induced tumor autophagy leading to the release of adenosine 5′-triphosphate capable of attracting and stimulating anti-tumor immune responses (Michaud, M. et al., Science, 334: 1573-1577). Overall the anti-tumor mechanism of action for combination treatment of the invention may not rely on the direct cytotoxic activity of the therapeutic agent. Therefore the combination treatment can be efficacious in subjects whose tumors are refractory to the therapeutic agent component.

### Kits

The invention provides kits for the treatment or prevention of neoplasia. In one embodiment, the kit includes a therapeutic or prophylactic composition containing a therapeutically effective amount of an IL-2 fusion protein in unit dosage form and one or more therapeutic agents. In preferred embodiments, the IL-2 fusion protein is ALT-801 and the one or more therapeutic agents are cisplatin and/or gemcitabine. In some embodiments, the kit comprises a sterile container which contains a therapeutic or prophylactic cellular composition; such containers can be boxes, ampoules, bottles, vials, tubes, bags, pouches, blister-packs, or other suitable container forms known in the art. Such containers can be made of plastic, glass, laminated paper, metal foil, or other materials suitable for holding medicaments.

If desired an IL-2 fusion protein and one or more therapeutic agents of the invention are provided together with instructions for administering the IL-2 fusion protein and one or more therapeutic agents to a subject having or at risk of developing cancer (e.g., bladder cancer). The instructions will generally include information about the use of the composition for the treatment or prevention of neoplasia. In other embodiments, the instructions include at least one of the following: description of the therapeutic agent; dosage schedule and administration for treatment or prevention of ischemia or symptoms thereof; precautions; warnings; indications; counter-indications; overdosage information; adverse reactions; animal pharmacology; clinical studies; and/or references. The instructions may be printed directly on the container (when present), or as a label applied to the container, or as a separate sheet, pamphlet, card, or folder supplied in or with the container.

### Recombinant Polypeptide Expression

The practice of the present invention employs, unless otherwise indicated, conventional techniques of molecular biology (including recombinant techniques), microbiology, cell biology, biochemistry and immunology, which are well within the purview of the skilled artisan. Such techniques are explained fully in the literature, such as, “Molecular Cloning: A Laboratory Manual”, second edition (Sambrook, 1989); “Oligonucleotide Synthesis” (Gait, 1984); “Animal Cell Culture” (Freshney, 1987); “Methods in Enzymology” (31) “Handbook of Experimental Immunology” (Weir, 1996); “Gene Transfer Vectors for Mammalian Cells” (Miller and Calos, 1987); “Current Protocols in Molecular Biology” (Ausubel, 1987); “PCR: The Polymerase Chain Reaction”, (Mullis, 1994); “Current Protocols in Immunology” (Coligan, 1991). These techniques are applicable to the production of the polynucleotides and polypeptides of the invention, and, as such, may be considered in making and practicing the invention. Particularly useful techniques for particular embodiments will be discussed in the sections that follow.

The following examples are put forth so as to provide those of ordinary skill in the art with a complete disclosure and description of how to make and use the assay, screening, and therapeutic methods of the invention, and are not intended to limit the scope of what the inventors regard as their invention.

### Examples

### Example 1: Intravenous Administration of a Novel IL-2 Fusion Protein, ALT-801, Inhibits Bladder Cancer in Mouse Models

ALT-801 is a fusion protein between interleukin-2 and a T cell receptor (TCR) domain capable of recognizing tumors presenting human p53 peptide (aa264-272)/HLA-A*0201 complexes. Intravenous administration of ALT-801 significantly prolonged survival of C57BL/6 mice bearing MB49luc orthotopic muscle invasive and superficial bladder cancer when compared with PBS treatment. The ALT-801-treated mice also survived rechallenge with MB49luc tumor cells, indicating long-lasting immune response and long-term memory. Additionally, ALT-801 exhibited potent antitumor activity against human bladder cancer HLA-A*0201⁺/p53⁺ UMUC-14 and HLA-A*0201-negative/p53⁺ KU7 xenografts in nude mice, which demonstrates that ALT-801's TCR domain targeting activity is not required for efficacy. ALT-801 combined with gemcitabine showed better antitumor effects and less toxicity than gemcitabine+cisplatin (GC) chemotherapy in the UMUC-14 and KU7 xenograft models, despite the different sensitivity of these tumor cells to GC.

### Example 2: Effect of ALT-801 in Combination with Gemcitabine and Cisplatin on Primary Tumor Growth of Human Bladder Cancer UMUC-14 in Nude Mice

The anti-tumor efficacy of multi-dose administration of c264scTCR-IL2 (ALT-801), alone and in combination with gemcitabine and cisplatin, was evaluated on primary tumor growth in athymic nude mice bearing human bladder UMUC-14 and KU7P cells. Treatment with a gemcitabine and cisplatin regimen is the standard-of-care for patients with metastatic bladder cancer. To assess the in vitro effects of these chemotherapeutic agents on human bladder cancer cells, HLA-A2⁺ p53⁺ UMUC-14 cells were treated with gemcitabine and cisplatin, alone and in combination. After a 24-hour incubation, gemcitabine, cisplatin, and gemcitabine+cisplatin caused a dose dependent decrease in UMUC-14 cell proliferation due to G0/G1 cell cycle arrest. These results are consistent with the mechanism of action of these agents on growing cells. In vitro incubation with the gemcitabine+cisplatin combination also induced presentation of the p53 peptide (aa264-272)/HLA-A*0201 complex on the surface of UMUC-14 tumor cells, indicating that the antigenic target for ALT-801 is elevated by this treatment.

The sensitivity of the human bladder tumor cell lines to gemcitabine and cisplatin was further assessed using a cell proliferation assay. UMUC-14 and KU7P cells were plated in media containing various amounts of gemcitabine and cisplatin and cell proliferation was determined using the WST-1 reagent 24 hours later. It was found that gemcitabine inhibited UMUC-14 cell growth with an IC₅₀ of 2030 μM whereas KU7P cell growth was inhibited at an IC50 of 0.05 μM. Cisplatin also showed much greater inhibition of KU7P cells (IC₅₀, 1.4 μM) than UMUC-14 cells (IC₅₀, 9.2 μM). Overall these results indicate that UMUC-14 cell growth is relatively resistant and KU7P cell growth is sensitive to the chemotherapeutic agents.

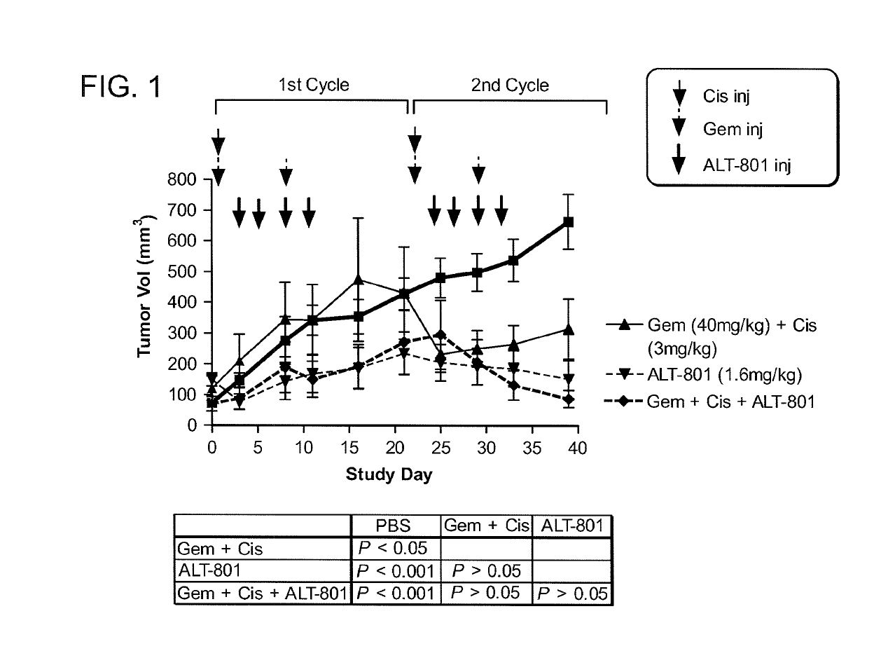

The antitumor effect of gemcitabine, cisplatin, and ALT-801 treatment was then evaluated in nude mice bearing subcutaneous UMUC-14 human bladder tumors. In this study, four groups of UMUC-14 tumor bearing mice (5 mice/group) were given two cycles of study drug treatment, each cycle lasting 3 weeks. For ALT-801 in combination with gemcitabine and cisplatin (Gem+Cis+ALT-801), cisplatin (Cis) (3 mg/kg) was given i.v. on study day 1 (SD1) and SD22, gemcitabine (Gem) (40 mg/kg) was given i.v. on SD1, SD8, SD22 and SD29, and ALT-801 (1.6 mg/kg) was given i.v. on SD3, SD5, SD8, SD10, SD24, SD26, SD29 and SD31 (FIG. 1). Other study treatment groups included ALT-801 monotherapy, Gem+Cis combination therapy, or PBS given on the appropriate schedule. Each of the three treatment regimens tested (Gem+Cis+ALT-801; ALT-801; Gem+Cis) resulted in a statistically significant decrease in growth of subcutaneous UMUC-14 human bladder tumors compared to that observed in PBS-treated mice (FIG. 1). Among the three treatment groups, Gem+Cis+ALT-801 showed the best efficacy, with a tumor growth inhibition (TGI) (relative to tumors in PBS-treated mice) of 87%, followed by ALT-801 (77% TGI) and Gem+Cis (52% TGI). The decrease in tumor volume seen with Gem+Cis treatment was only observed during the second cycle of treatment and may have been attributed in part to breakage or necrosis of the large tumors rather than direct anti-tumor activity. ALT-801 in combination with gemcitabine and cisplatin treatment did not significantly reduce the mouse body weight and there was no observed mortality or post-treatment signs of toxicity, suggesting the treatment regimen was safe.

### Example 3: Effects of ALT-801 or MART-1scTCR/IL-2 Fusion Proteins in Combination with Gemcitabine on Primary Tumor Growth of UMUC-14 Human Bladder Cancer in Nude Mice

This study was conducted as a follow-up to evaluate the anti-tumor efficacy of multi-dose administration of ALT-801 (c264scTCR-IL2) plus gemcitabine and a non-targeted scTCR/IL-2 fusion protein (MART-1scTCR/IL-2) plus gemcitabine on primary tumor growth in athymic nude mice bearing human bladder UMUC-14 cells. ALT-801 (c264scTCR/IL-2) recognizes tumor cells displaying the p53 (aa264-272)/HLA-A*0201 complex and has been demonstrated to inhibit growth of HLA-A*0201⁺/p53⁺ subcutaneous tumors in athymic nude mice (Belmont, et al. 2006 Clin Immunol. 121:29, Wen, et al. 2008 Cancer Immunol Immunother. 57:1781). MART-1scTCR/IL-2, a different scTCR/IL-2 fusion protein, recognizes the MART-1 (aa27-35) peptide presented in the context of HLA-A*0201 but not p53 (aa264-272)/HLA-A*0201. This protein has served as a non-targeted control reagent in studies with HLA-A*0201⁺/p53⁺ subcutaneous tumors. ALT-801 and MART-1scTCR/IL-2 exhibited equivalent abilities to bind cell-surface IL-2 receptors and stimulate NK cell responses. However, ALT-801 exhibited much better anti-tumor activity than MART-1scTCR/IL-2 against subcutaneous HLA-A*0201⁺/p53⁺ A375 human melanoma tumors in mouse model (Wen, et al. 2008 Cancer Immunol Immunother. 57:1781). This effect is likely due to tumor specific recognition by the ALT-801 protein.

The efficacy of ALT-801 and MART-1scTCR/IL-2, in combination with gemcitabine, was evaluated to determine the contribution of tumor targeting to the anti-tumor activity of the scTCR/IL-2 fusion proteins. Tumor-bearing mice receiving gemcitabine plus cisplatin served as a control group for this study.

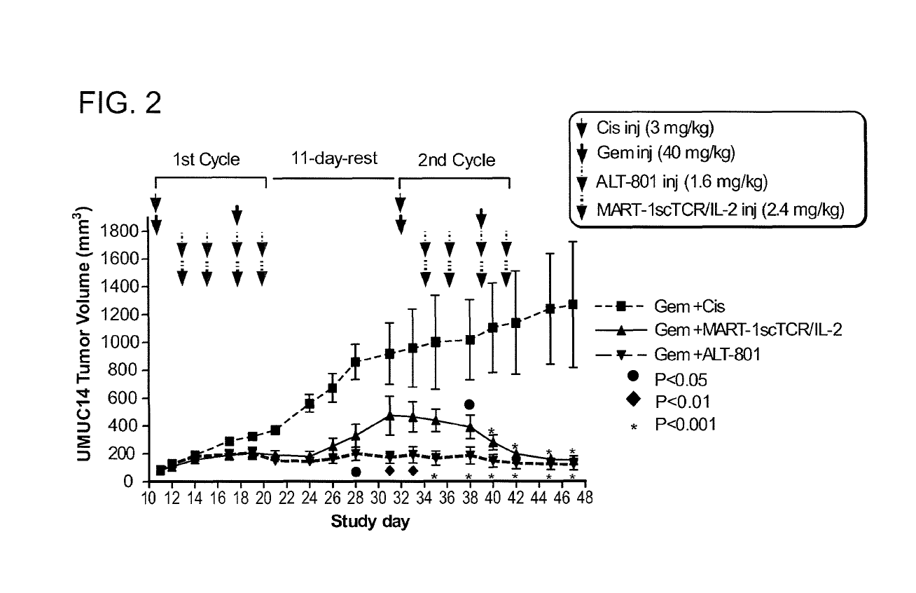

Athymic nude mice (4 animals/group) bearing subcutaneous UMUC-14 tumors (average volume 80 mm³) were treated intravenously (i.v.) with gemcitabine (40 mg/kg) (Gem) plus cisplatin (3 mg/kg) (Cis), ALT-801 (1.6 mg/kg) plus Gem (40 mg/kg) or MART-1scTCR/IL-2 (2.4 mg/kg, dose equivalent activity of ALT-801) plus Gem (40 mg/kg), given for two cycles of treatment. The first treatment cycle consisted of 1 Cis injection on Study Day (SD) 1, two Gem injections on SD 1 and SD 8, and four injections of ALT-801 or MART-1 scTCR/IL-2 on SD 3, SD 5, SD 8 and SD 10 in the first cycle. After an 11-day rest period (SD 15-SD 21), a second cycle of treatment was conducted for this study using the same regimen as in the first cycle followed by a 6-day follow-up period (SD42-SD47). The treatment using ALT-801+Gem or MART-1scTCR/IL-2+Gem resulted in a statistically significant decrease in growth of subcutaneous UMUC-14 human bladder tumors compared to that observed in Gem+Cis-treated mice (FIG. 2). Overall no significant difference in anti-tumor activity was found between ALT-801+Gem and MART-1scTCR/IL-2+Gem treatment, although ALT-801+Gem showed a trend of better anti-tumor efficacy during the treatment course. These results confirm the previous results demonstrating the potent anti-tumor activity of ALT-801 treatment regimens in this model. Additionally, the observed efficacy of the non-targeted MART-1scTCR/IL-2 fusion protein indicates that UMUC-14 human bladder xenografts are also highly sensitive to IL-2 based therapies. Therefore, this data demonstrates that the targeting activity of the c264scTCR component of ALT-801 is not required for its potent efficacy against the UMUC-14 bladder tumor cells. Together with Example 2, the results clearly indicate that the combination of an IL-2 fusion protein with chemotherapy (either gemcitabine+cisplatin or gemcitabine) resulted in effective treatment against human bladder tumors, including tumor cells that are resistant to the chemotherapeutic agents.

There was no observed mortality or post-treatment sign of toxicity during treatment regimen. At several time points during the treatment course, significant body weight loss was observed in the ALT-801+Gem and MART-1scTCR/IL-2+Gem treatment groups compared to animals treated with Gem+Cis. However, mean mouse body weights for both the ALT-801+Gem and MART-1scTCR/IL-2+Gem treatment groups recovered rapidly during the 11-day rest period and one-week follow-up period. These findings demonstrate that the ALT-801+Gem and MART-1 scTCR/IL-2+Gem treatment regimens are well tolerated with transient toxicities in this model.

### Example 4: Effects of ALT-801 or MART-1scTCR/IL-2 Fusion Proteins in Combination with Gemcitabine on Primary Tumor Growth of UMUC-14 and KU7 Human Bladder Cancer in Nude Mice

These studies were conducted to evaluate the anti-tumor efficacy of multi-dose administration of ALT-801 (c264scTCR/IL-2), in combination with gemcitabine or gemcitabine and cisplatin and a non-targeted scTCR/IL-2 fusion protein (MART-1scTCR/IL-2) in combination with gemcitabine on primary tumor growth in athymic nude mice bearing human bladder UMUC-14 or KU7P cells. Tumor-bearing mice receiving PBS or Gem plus Cis served as control groups for this study. Gem and Cis is the standard-of-care chemotherapy for patients with metastatic bladder cancer.

Athymic nude mice (5 animals/group) bearing subcutaneous UMUC-14 tumors (average volume 84 mm³) were treated with PBS, Gem (40 mg/kg) plus Cis (3 mg/kg), MART-1scTCR/IL-2 (2.19 mg/kg, dose equivalent activity of ALT-801) plus Gem (40 mg/kg), ALT-801 (1.6 mg/kg) plus Gem (40 mg/kg) or ALT-801 (1.6 mg/kg) plus Gem (40 mg/kg) and Cis (3 mg/kg), given for two cycles of treatment. The first treatment cycle consisted of 1 Cis injection on Study Day (SD) 9, two Gem injections on SD 9 and SD 16, and four injections of ALT-801 or MART-1scTCR/IL-2 on SD 11, SD 13, SD 16 and SD 18 in the first cycle. After an 11-day rest period (SD 19-SD 30), a second cycle of treatment was conducted for this study using the same regimen as in the first cycle followed by a 10-day follow-up period (SD40-SD49).

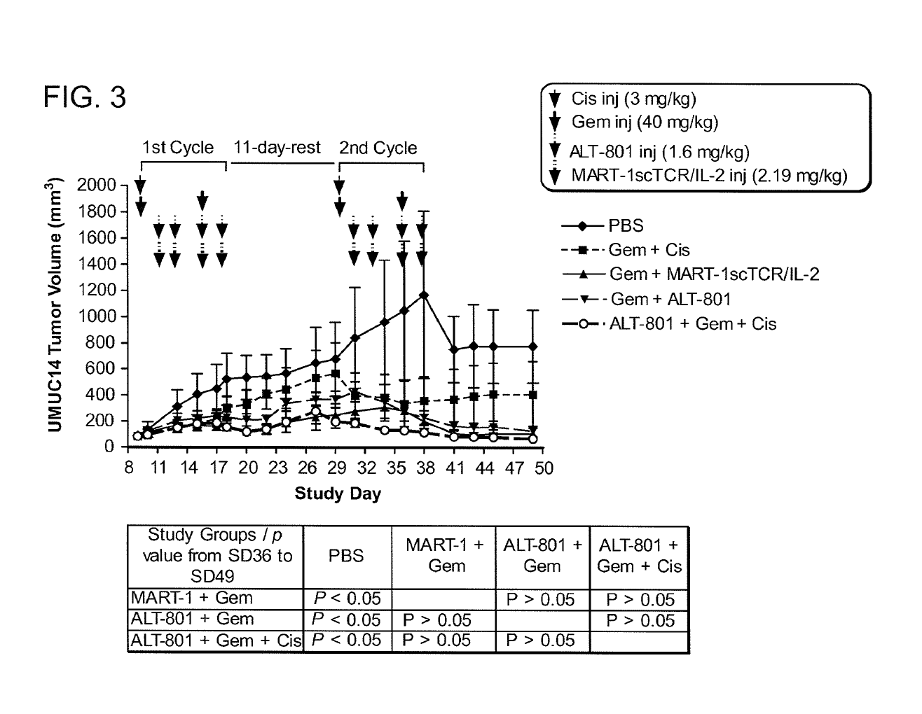

The treatment using MART-1scTCR/IL-2+Gem, ALT-801+Gem or ALT-801+Gem+Cis resulted in a statistically significant decrease in growth of subcutaneous UMUC-14 human bladder tumors compared to that observed in PBS treated mice (FIG. 3). This statistically significant decrease in growth was observed even though some surfaces of tumors were cracked which markedly affected the accuracy of tumor volume measurements in both PBS (starting on SD38) and Gem+Cis (starting on SD29) groups. No significant difference in anti-tumor activity was found among the treatment groups, MART-1scTCR/IL-2+Gem, ALT-801+Gem, and ALT-801+Gem+Cis, although ALT-801+Gem+Cis showed a trend of better anti-tumor efficacy during the treatment course of the current study. These results confirm the previous results demonstrating the potent anti-tumor activity of ALT-801 treatment regimens in this animal model. Additionally, the observed efficacy of the non-targeted MART-1scTCR/IL-2 fusion protein indicates that UMUC-14 human bladder xenografts are also highly sensitive to IL-2 based therapies. Therefore, this data further demonstrates that the targeting activity of the 264scTCR component of ALT-801 is not required for its potent efficacy against the UMUC-14 bladder tumor cells.

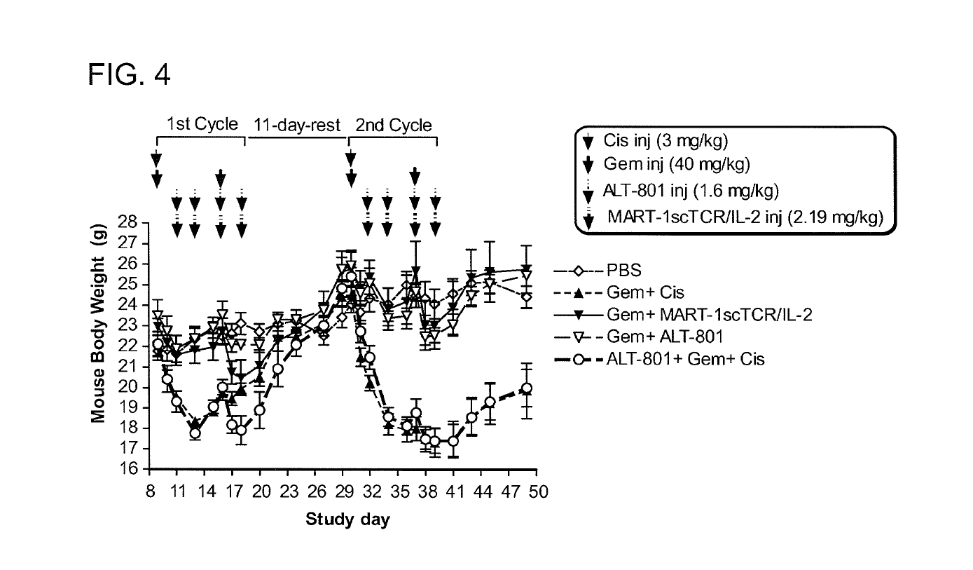

There was no observed mortality during the treatment regimen. However, at several time points during the treatment course, significant body weight loss was observed in the Gem+Cis and ALT-801+Gem+Cis treatment groups compared to animals not treated with Cis (FIG. 4). No significant difference in anti-tumor activity was found by using Cis and the recovery from the weight loss was slow indicating a higher toxicity of Cis in this model. These results show that Cis does not provide therapeutic benefit in this treatment. Body weight loss was also found in both ALT-801+Gem and MART-1scTCR/IL-2+Gem treatment groups when compared to PBS group, however, mean mouse body weights for both the ALT-801+Gem and MART-1scTCR/IL-2+Gem treatment groups recovered rapidly during the 11-day rest period and 13-day follow-up period. These findings demonstrate that the ALT-801+Gem and MART-1scTCR/IL-2+Gem treatment regimens are well tolerated with transient toxicities in this model.

As a follow-up, a different human bladder tumor cell line, KU7P, was used to further evaluate the efficacy of ALT-801 and MART-1scTCR/IL-2, in combination with Gem or Gem+Cis. This cell line is a HLA-A*0201 negative and p53 overexpressing cell line and does not display antigens recognized by either the ALT-801 or MART-1scTCR/IL-2 molecules. Thus, the results of this model could provide further evidence that the “non-targeted” anti-tumor activity of scTCR/IL-2 fusions in combination with Gem is efficacious against primary human bladder tumor xenografts in nude mice. Tumor-bearing mice receiving PBS or Gem+Cis served as a control group for this study. Athymic nude mice (5 animals/group) bearing subcutaneous KU7P tumors (average volume of 81 mm³ except for the PBS group [˜0.70 mm³]) were treated with PBS, Gem (40 mg/kg) plus Cis (3 mg/kg), MART-1scTCR/IL-2 (2.19 mg/kg, dose equivalent activity of ALT-801) plus Gem (40 mg/kg), ALT-801 (1.6 mg/kg) plus Gem (40 mg/kg), or ALT-801 (1.6 mg/kg) plus Gem (40 mg/kg) and Cis (3 mg/kg), given for two cycles of treatment. The first treatment cycle consisted of 1 Cis injection on Study Day (SD) 7, two Gem injections on SD 7 and SD 14, and four injections of ALT-801 or MART-1scTCR/IL-2 on SD 9, SD 11, SD 14 and SD 16 in the first cycle. After an 11-day rest period (SD 17-SD 27), a second cycle of treatment was conducted for this study using the same regimen as in the first cycle followed by a 10-day follow-up period (SD37-SD45).

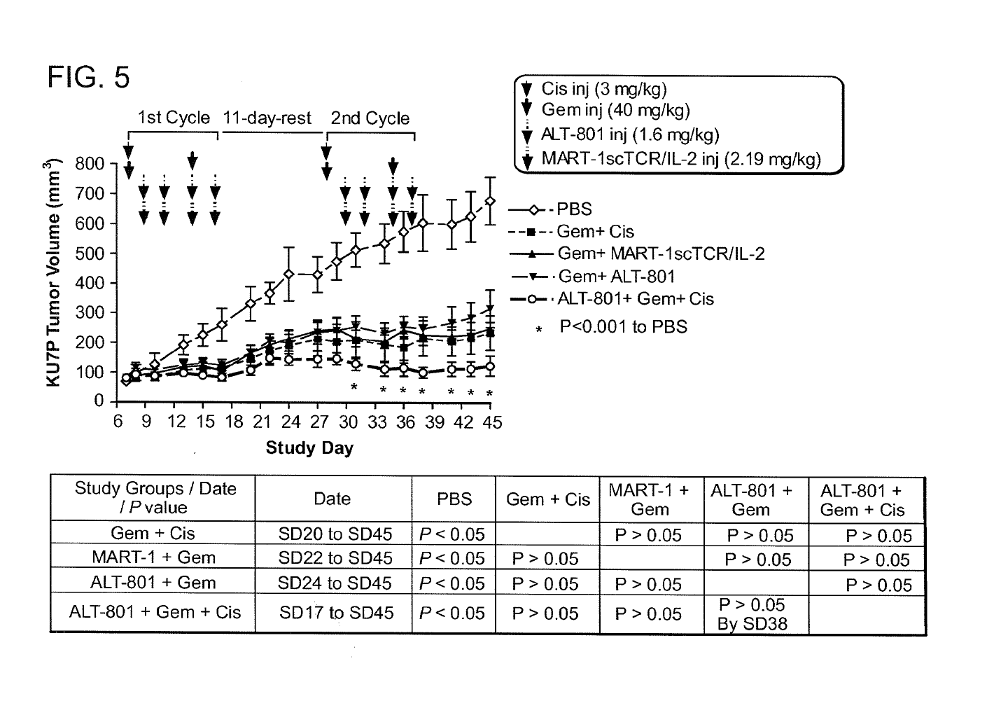

The treatment using Gem+Cis, MART-1 scTCR/IL-2+Gem, ALT-801+Gem or ALT-801+Gem+Cis resulted in a statistically significant decrease in growth of subcutaneous KU7P human bladder tumors compared to that observed in PBS treated mice (FIG. 5). Overall no statistically significant difference in anti-tumor activity was found among the treatment groups, Gem+Cis, MART-1scTCR/IL-2+Gem, ALT-801+Gem, and ALT-801+Gem+Cis, although ALT-801+Gem+Cis showed a trend toward better anti-tumor efficacy (i.e. compared to Gem+Cis) during the treatment course. These results are consistent with the above referenced study results demonstrating the potent anti-tumor activity of ALT-801 and MART-1scTCR/IL-2 treatment regimens in UMUC-14 bladder tumor xenograft model. Additionally, the observed non-targeted efficacy of ALT-801 and MART-1scTCR/IL-2 in combination with Gem on HLA-A*0201-negative/p53 overexpressing KU7P human bladder tumors in nude mice indicate that KU7P human bladder xenografts are highly sensitive to Gem+IL-2 based therapies. Therefore, this data further demonstrates that the targeting activity of the 264scTCR component of ALT-801 is not required for the potent efficacy in combination with Gem against the KU7P bladder tumor cells. The results of this study also indicate that the Gem+Cis regimen was more efficacious at inhibiting growth of KU7P bladder tumors than it was in the UMUC-14 bladder tumor model and ALT-801 is efficacious against these bladder cancer cells without regard to their sensitivity to the Gem+Cis regimen. These results are consistent with the in vitro sensitivity of KU7P cells and resistance of UMUC-14 cells to gemcitabine and cisplatin described above. Treatment with the IL-2 fusion proteins ALT-801 or MART-1scTCR/IL-2 alone or in combination with Gem+Cis was found to be efficacious against both chemotherapy sensitive and resistant bladder tumors.

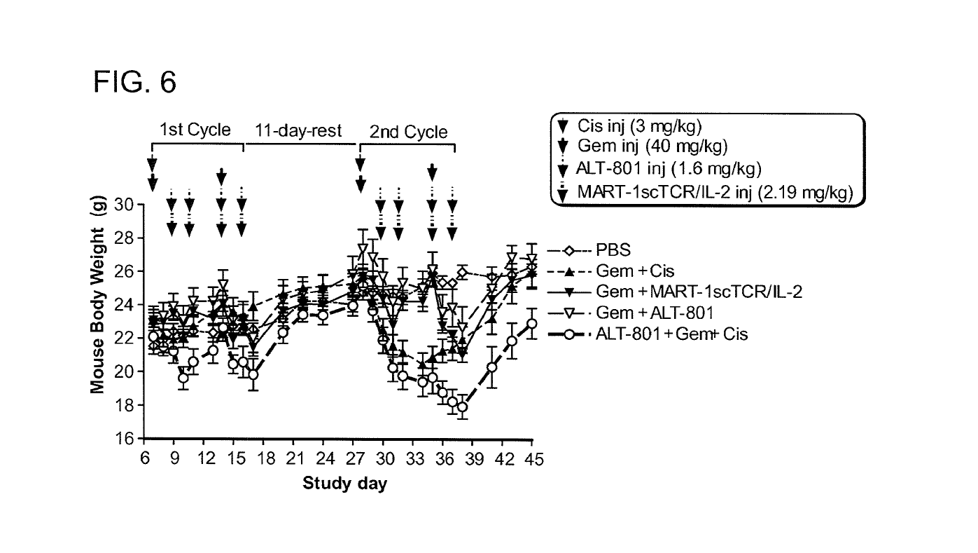

There was no observed mortality during the treatment regimen. However, consistent with the above referenced study, significant body weight loss was observed in the Gem+Cis and ALT-801+Gem+Cis treatment groups compared to animals not treated with Cis (FIG. 6). As indicated above, KU7P bladder tumors are sensitive to Cis and exhibit a slightly better anti-tumor activity when administered a combination of ALT-801+Gem. However the recovery from the weight loss in the Cis treatment groups was slow indicating a higher toxicity of Cis and, therefore, an unfavorable therapeutic index in this model. Body weight loss was also found in both ALT-801+Gem and MART-1scTCR/IL-2+Gem treatment groups when compared to PBS group, especially in 2nd treatment cycle, however, mean mouse body weights for both the ALT-801+Gem and MART-1scTCR/IL-2+Gem treatment groups recovered rapidly during the 11-day rest period and 8-day follow-up period. These findings suggest that the ALT-801+Gem and MART-1scTCR/IL-2+Gem treatment regimens are well tolerated with transient toxicities in this model.

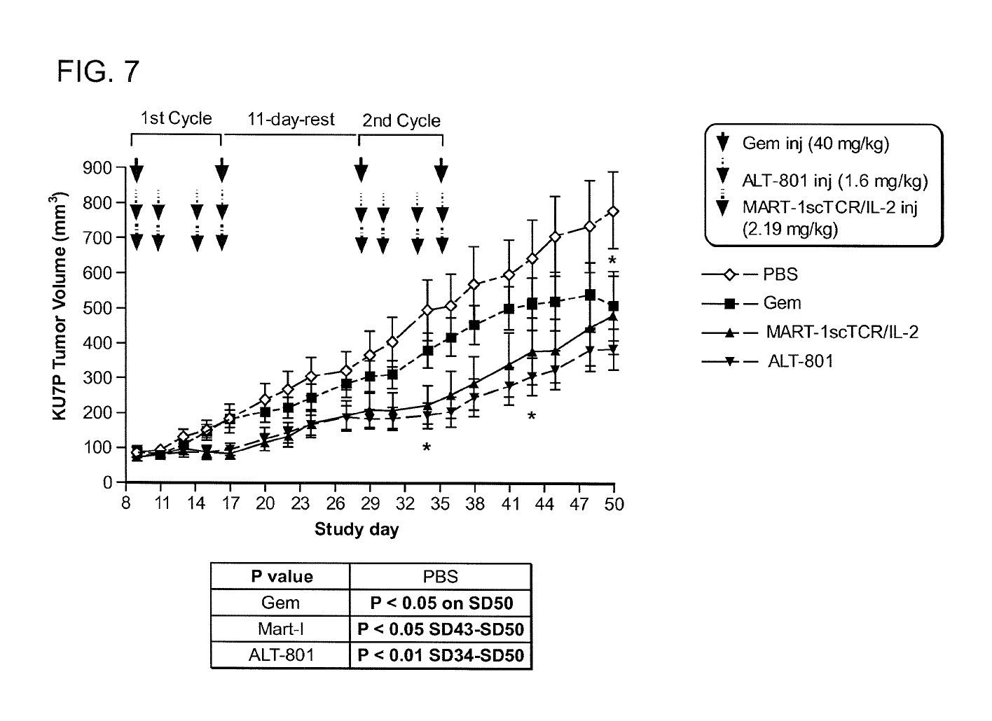

A similar study was conducted in the subcutaneous KU7P bladder tumor xenograft model to examine the anti-tumor effects of monotherapy with Gem (40 mg/kg), MART-1scTCR/IL-2 (2.19 mg/kg, dose equivalent activity of ALT-801), or ALT-801 (1.6 mg/kg) using the same treatment schedule described above. As shown in FIG. 7, treatment with Gem, MART-1scTCR/IL-2 or ALT-801 resulted in a statistically significant decrease in growth of subcutaneous KU7P human bladder tumors compared to that observed in PBS treated mice. This effect appeared to be less durable than that observed with the Gem+MART-1scTCR/IL-2 and Gem+ALT-801 combinations where little or no regrowth of the tumors was seen following succession of treatment (FIGS. 3 and 5). Thus combination treatment with an IL-2 fusion protein and chemotherapy (in this case gemcitabine) appeared to provide the most effective anti-tumor activity against human bladder tumors.

### Example 5: Effect of ALT-801 on Survival of C57BL/6 and Albino C57BL/6 Mice Bearing MB49luc Orthotopic Muscle Invasive Bladder Tumors. The Targeting Activity of ALT-801's TCR Domain is not Necessary for Anti-Tumor Activity

The effects of multi-dose administration of ALT-801 (c264scTCR-IL2) were evaluated on the survival of immunocompetent C57BL/6 and albino C57BL/6 mice bearing syngeneic MB49luc orthotopic muscle invasive bladder tumors. Because these tumors lack the p53 (aa264-272)/HLA-A*0201 complex recognized by ALT-801, this study is designed to assess the “non-targeted” anti-tumor activity of ALT-801 against bladder cancer.

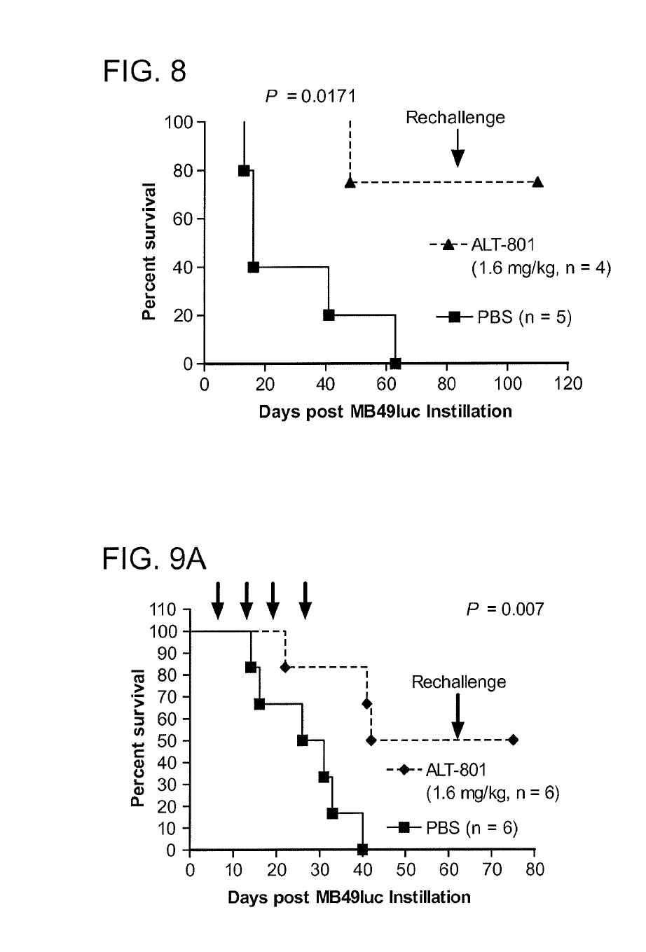

A relevant and reproducible mouse bladder cancer model (murine bladder cancer cell line MB49luc) in immunocompetent albino C57BL/6 mice was used to evaluate the efficacy of ALT-801. The MB49luc cell line expresses luciferase allowing for its detection using a bioluminescence assay. Following trypsin-EDTA pretreatment of the bladders, MB49luc (1×10⁶ cells/bladder) were instilled intravesically into the bladders of albino C57BL/6 mice (17 weeks old) on study day 0. PBS (n=5) or ALT-801 (1.6 mg/kg, n=4) was administered i.v. on 9, 16, 23, and 30 days post MB49luc tumor cell instillation. The mice were maintained to assess the survival rate among the treatment groups after tumor instillation as the efficacy endpoint. ALT-801 significantly prolonged the survival of the MB49luc bearing mice compared with PBS (P=0.0171) (FIG. 8). Surviving animals in the ALT-801 treatment group were re-challenged with intravesical instillation of MB49luc cells (1×10⁶ cells per mouse) 84 days after initial instillation. Additionally naïve C57BL/6 control mice were instilled with the tumor cells on the same day to serve as a control. Luciferase-based imaging to detect MB49luc cells was then performed 16 days after re-challenge with MB49luc cells. Tumor cell re-challenged mice of the previously ALT-801-treated group showed no bioluminescence tumor signals, whereas the MB49luc-instilled naïve mice showed evidence of tumor cell signal, demonstrating that the mice of the previously ALT-801-treated group were resistant to re-implantation of the MB49luc tumor cells. Kaplan-Meier survival curves showed that the re-challenged mice of the previously ALT-801-treated group survived significant longer than naïve MB49luc-instilled mice (P=0.0246).

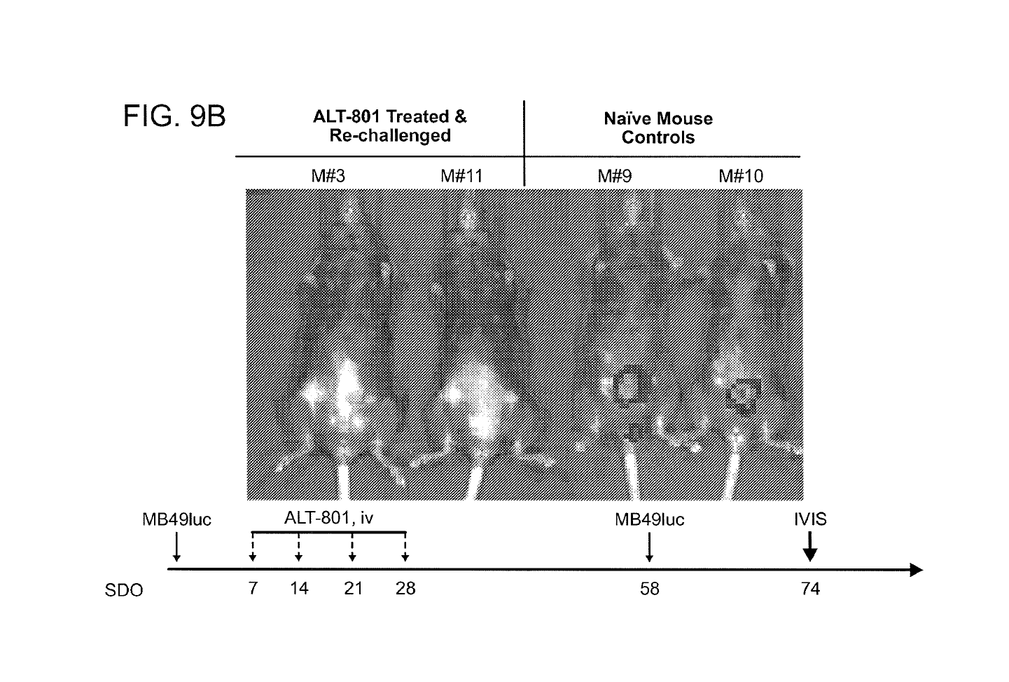

Similarly, in another experiment, C57BL/6 mice (9-10 weeks old) were instilled intravesically with MB49luc (0.075×10⁶ cells/bladder) on study day 0, following polylysine pretreatment of the bladders. PBS (n=6) or ALT-801 (1.6 mg/kg, n=6) was administered i.v. on 7, 14, 21, and 28 days post MB49luc tumor cell instillation. The mice were maintained to assess survival rate among the treatment groups as the efficacy endpoint. ALT-801 significantly prolonged the survival of the MB49luc bearing mice compared with PBS (P=0.007) (FIG. 9A). The survivors of the ALT-801 treatment were re-challenged with intravesical instillation of MB49luc cells (0.075×10⁶ cells per mouse) 62 days after initial instillation. Additionally naïve C57BL/6 control mice (n=2) were instilled with the tumor cells on the same day to serve as a control. Imaging was then performed 16 days after re-challenge with MB49luc cells. Tumor cell re-challenged mice of the previously ALT-801-treated group showed no bioluminescence tumor signals, whereas the MB49luc-instilled naïve mice showed evidence of tumor cell signal, suggesting that the mice of previously ALT-801-treated group were resistant to re-implantation of the MB49luc tumor cells (FIG. 9B). Mice of the previously ALT-801-treated group survived longer than the naïve mice after re-challenge, although Kaplan-Meier analysis did not show statistical significance in survival time between the two groups (P=0.0896), probably due to small numbers of mice used per group.

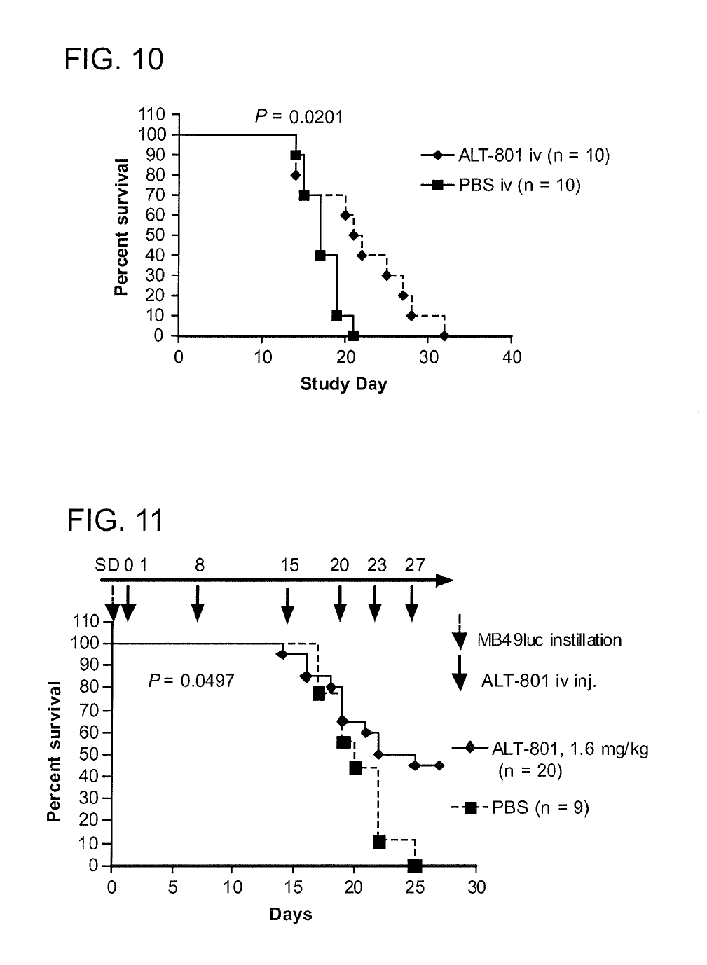

In additional studies, the efficacy of intravenous administration of ALT-801 was further evaluated in an orthotopic MB49luc muscle invasive bladder cancer model in immunocompetent C57BL/6 mice. Following polylysine pretreatment of the bladders, MB49luc (0.075×10⁶ cells/bladder in 100 μL) were instilled intravesically into the bladders of C57BL/6 mice (10-11 weeks old) on study day 0. PBS (n=10) or ALT-801 (1.6 mg/kg, n=10) was administered i.v. on 7, 14, 21, and 28 days post MB49luc tumor cell instillation. The mice were maintained to assess survival rate among the treatment groups after tumor instillation as the efficacy endpoint. ALT-801 significantly prolonged the survival of the MB49luc bearing mice compared with PBS (P=0.0201) (FIG. 10). Consistent with previous studies in this model, the observed anti-tumor effects of ALT-801 against orthotopic MB49luc muscle invasive bladder tumors are independent of the antigen targeting activity of the ALT-801 fusion protein.

In sum these results demonstrated that ALT-801 i.v. treatment was effective in prolonging survival time of immunocompetent mice bearing syngeneic MB49luc orthotopic muscle invasive bladder tumors. ALT-801 treatment also provides durable immunological memory response against tumors to which they were previously exposed. These effects are independent of the targeting activity of the ALT-801 fusion protein.

### Example 6: Intravenous Administration of ALT-801 Prolonged Survival of C57BL/6 Mice Bearing MB49luc Orthotopic Superficial Bladder Tumors

This study was conducted to evaluate the effect of ALT-801 when administered via intravenous (i.v.) injection in multi-dose regimens on survival of C57BL/6 mice bearing murine MB49luc orthotopic superficial bladder tumors. This study employed the relevant and reproducible murine MB49luc bladder cancer model in immunocompetent C57BL/6 mice described in the previous Examples. It has been demonstrated that intravesical instillation of MB49luc cells into the bladders of C57BL/6 mice resulted in a superficial form of bladder cancer 1-2 days post instillation. The tumors advanced to the muscle invasive form by day 7-9 post instillation and tumor-mediated mortality was observed after 2-3 weeks. In the present study, the survival benefit of i.v. administration of ALT-801 was examined in C57BL/6 mice bearing murine orthotopic superficial bladder cancer derived from MB49luc cells. Since these tumors lack the human p53 (aa264-272)/HLA-A*0201 complex recognized by ALT-801, this study is designed to assess the “non-targeted” anti-tumor activity of ALT-801 against mouse orthotopic superficial bladder cancer.

MB49luc (0.075×10⁶ cells/bladder in 100 μL) were instilled intravesically into the bladders of C57BL/6 mice (9-11 weeks old) on study day 0 following polylysine pretreatment of the bladders. PBS (n=8) or ALT-801 (1.6 mg/kg, n=20) was administered i.v. on 1, 8, 15, 20, 23 and 27 days post tumor cell instillation. Intravenous administration of ALT-801 significantly prolonged the survival of mice when compared with the PBS control (P=0.0497) (FIG. 11).

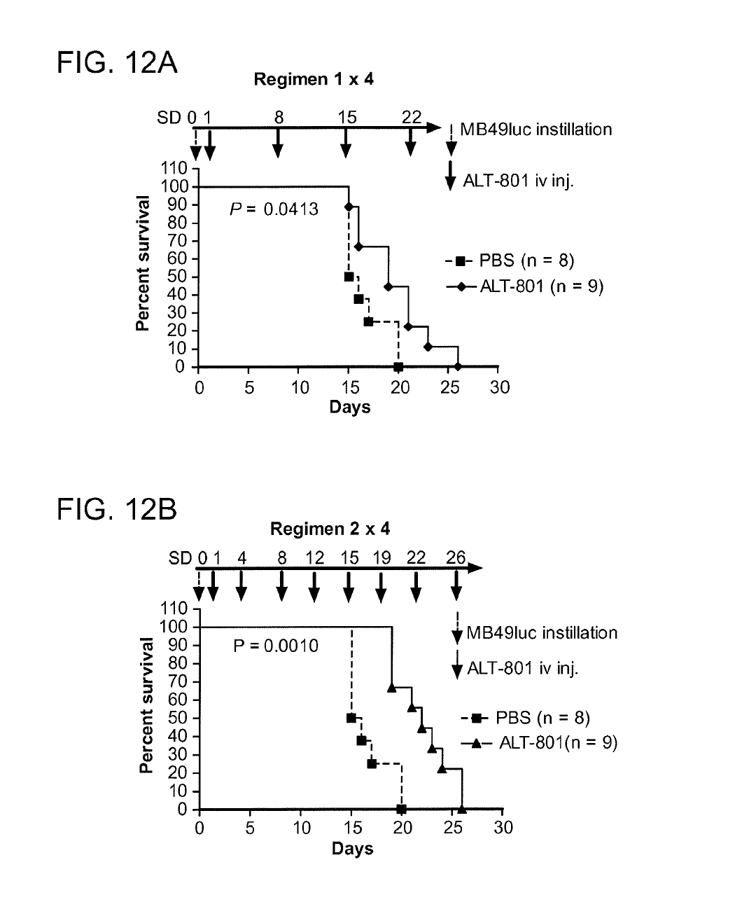

In another experiment, C57BL/6 mice (9-11 weeks old) were instilled intravesically with MB49luc cells (0.075×10⁶ cells/bladder) on study day 0, following polylysine pretreatment of the bladders. One group of mice (ALT-801 “1×4”, n=9) was treated intravenously via the lateral tail vein with four weekly ALT-801 injections at 1.6 mg/kg on SD1, SD8, SD15, SD22, a second group of mice (ALT-801 “2×4”, n=9) was treated with eight ALT-801 injections at 1.6 mg/kg (twice weekly for four weeks) on SD1, SD4, SD8, SD12, SD15, SD19, SD22, and SD26, and a control group (n=8) was treated with PBS (100 μL) on SD1, SD4, SD8, SD12, SD15, SD19, SD22, and SD26 post-tumor instillation. Both “1×4” and “2×4” treatment regimens with ALT-801 at 1.6 mg/kg significantly prolonged mouse survival when compared with PBS control group (P=0.0413 and P=0.0010, respectively). The median survival times for PBS, ALT-801 “1×4” (FIG. 12A) and ALT-801 “2×4” (FIG. 12B) groups were 15.5, 19 and 22 days, respectively. The results suggest that twice weekly administration of ALT-801 provides the best anti-tumor activity against murine MB49luc orthotopic superficial bladder cancer. The observed anti-tumor effects of the test article are independent of the antigen targeting activity of the ALT-801 fusion protein.

### Example 7: Immune Cells Induced by ALT-801 Treatment of C57BL/6 Mice Bearing MB49luc Orthotopic Bladder Tumors

This study was conducted to evaluate the immune cell-based mechanism of action of ALT-801 treatment in C57BL/6 mice bearing murine MB49luc orthotopic bladder tumors. As described above, C57BL/6 mice were instilled intravesically with MB49luc cells (0.075×10⁶ cells/bladder) on study day (SD) 0, following polylysine pretreatment of the bladders. Mice without tumor instillation served as a control. The mice (6/group) were then treated i.v. with PBS or ALT-801 (1.6 mg/kg) on SD 7, 10, 14 and 17. Three days after each treatment (i.e., SD 10, 13, 17, 20), groups of mice were sacrificed, the bladders were examined for tumor progression (hematuria, bladder size, appearance, neovascularization, and morphology) and the blood, spleens and bladders were collected for immune cell analysis. PBMCs were prepared from the blood, splenocytes suspensions were prepared from the spleens and the bladders were fixed and sectioned for immunohistochemical staining. Immune cells (CD3, NK and CD8 positive cells) in the PMBCs and splenocytes were stained with monoclonal antibodies and analyzed by flow cytometry. Immune cells (macrophage, NK and CD3 positive cells) were assessed in bladder sections by IHC and tumor cells were examined by H&E staining. Additionally throughout the study, urine was collected from the animals to assess urine cytokine levels (IFNγ and TNFα) by ELISA.

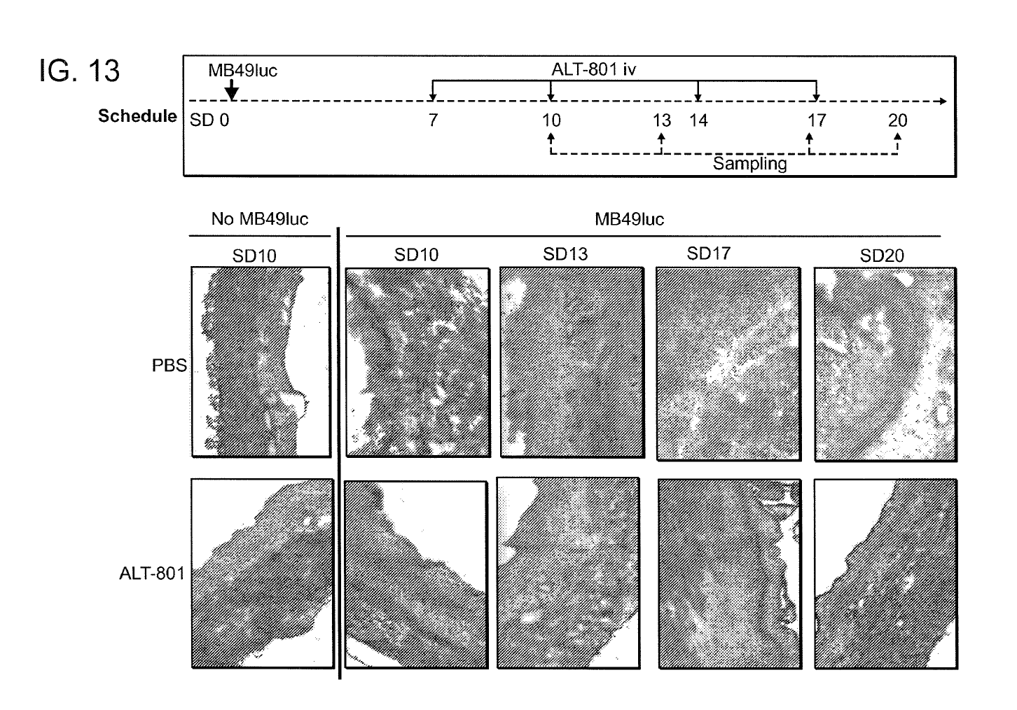

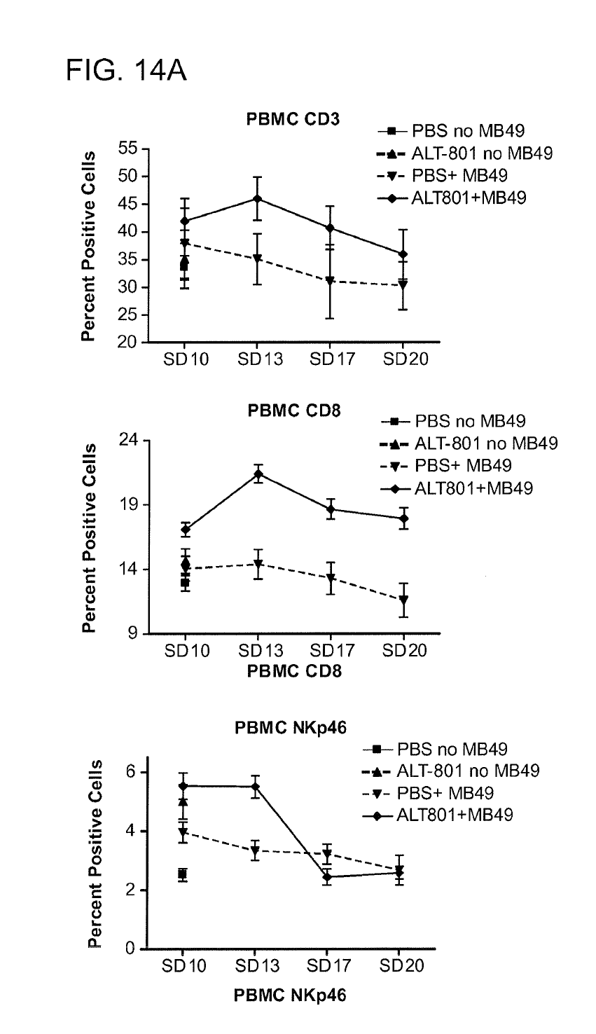

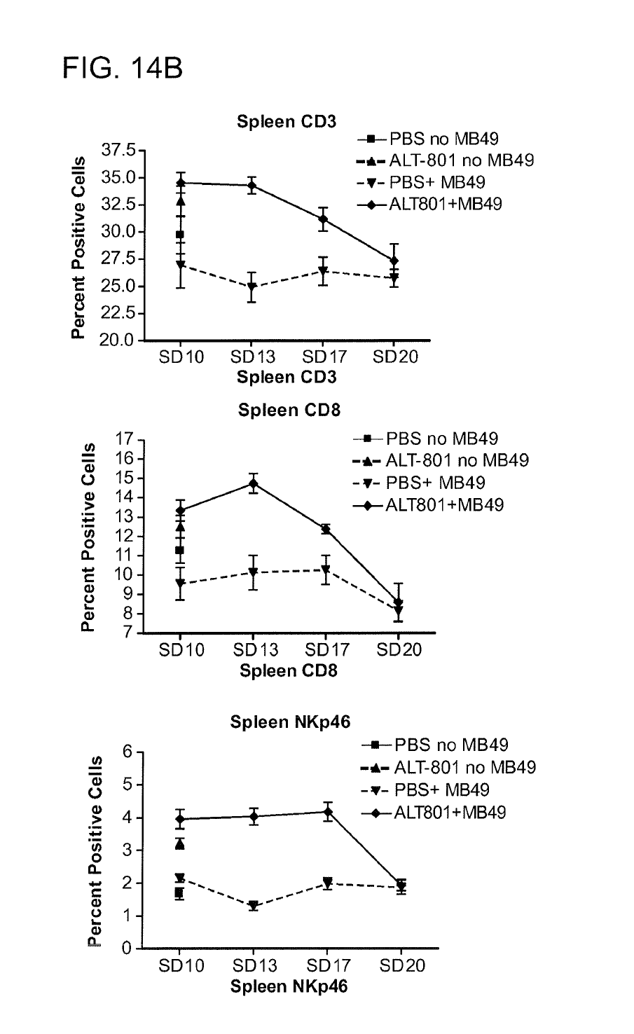

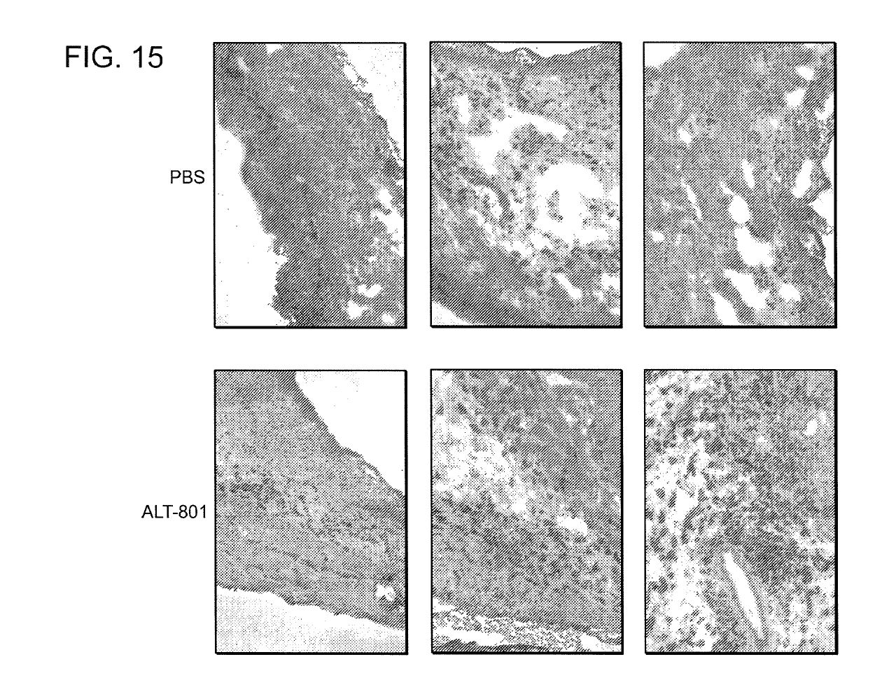

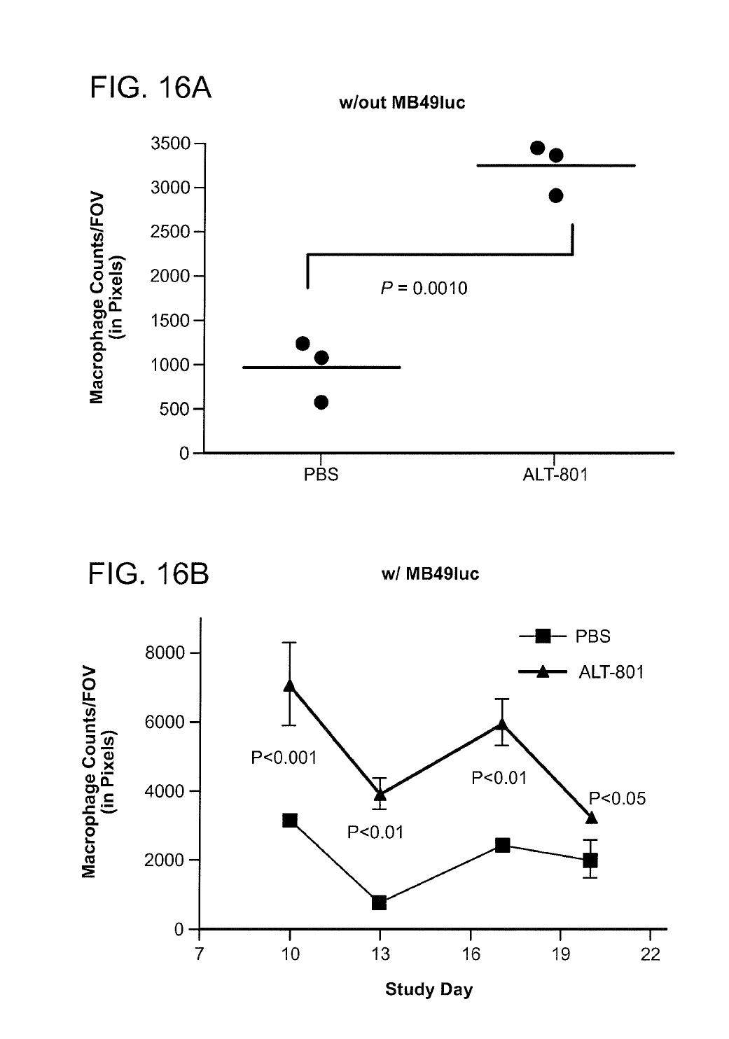

Similar to previous studies, intravesicular instillation of MB49luc cells resulted in establishment of orthotopic tumors in the bladder and rapid progression of these tumors into the muscular layer within 7 to 20 days (FIG. 13). These changes were reflected by increased hematuria, neovascularization of the bladder and increases in bladder size and other changes in appearance. As in previous studies, treatment with ALT-801 reversed these changes resulting in normal appearing bladders by SD20 (FIG. 13). However it is noteworthy that ALT-801 treatment of either MB49luc tumor bearing or normal mice resulted in an increase in immune cell infiltration into the bladder. These changes were also reflected in the PBMCs and spleens where ALT-801 treatment resulted in increases in CD3, CD8 and NK cells in both MB49luc tumor-bearing or normal mice (FIG. 14A & 14B). With continued ALT-801 treatment, the induced immune cells (except for PBMC CD8 cells) returned to normal levels by SD20. In the bladders, macrophage levels were most obviously affected by ALT-801 treatment particularly at SD 10 in tumor bearing animals (FIG. 15). The levels of stained macrophage in the bladder sections were quantitated and the graphed data is shown in FIG. 16. The results indicate that both in normal mice (FIG. 16A) and MB49luc-tumor bearing mice (FIG. 16B), ALT-801 treatment resulted in significant increases bladder macrophage levels. In the tumor bearing mice, the induced macrophage levels returned to near normal levels by SD20 as the bladder morphology returned to normal. Similar but less significant changes in NK and CD3 positive cells were also seen in ALT-801 treated mice. These results suggest that ALT-801 induction of macrophage and other immune cell subtypes in the bladder are responsible for the anti-bladder tumor effects observed in this model.

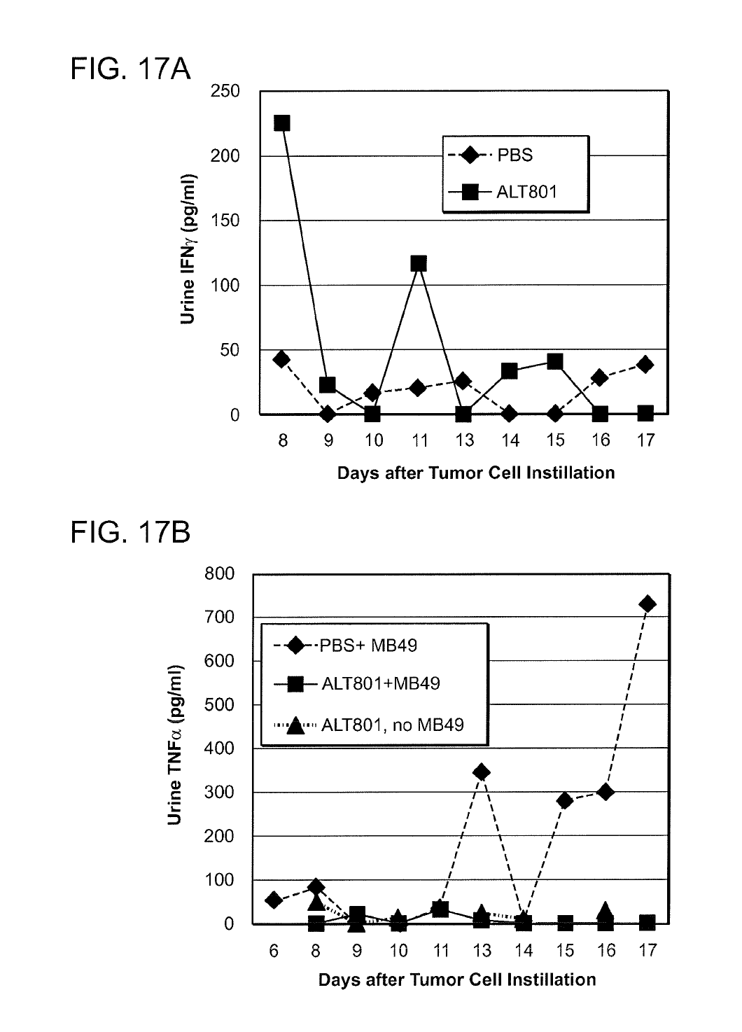

Analysis of cytokine levels in the urine also indicated that ALT-801 treatment resulted in stimulation of immune responses. After each dose of ALT-801, an increase IFNγ levels was detected (FIG. 17A) in urine from MB49luc-tumor bearing mice. ALT-801 treatment did not induce TNFα levels in the urine (FIG. 17B) of these animals. However, TNFα levels increase in PBS treated tumor bearing mice over time, suggesting a causal relationship between tumor growth and urine TNFα levels. ALT-801-mediated induction of serum IFNγ and a lack of a treatment effect on serum TNFα levels was observed in cancer patients (Fishman et al. (2011) Clin Cancer Research 17:7765), indicating this is a common immune response to ALT-801 treatment. Together these observations support role of IFNγ-producing immune cells, possibly macrophage, in the anti-tumor activity observed following ALT-801 treatment.

### Example 8: ALT-801 Increased Survival of Mice in a Murine Model of Multiple Myeloma

To investigate the effects and mechanism-of-action of the fusion protein, ALT-801 (c264scTCR-IL2), was administered as a multi-dose regimen in an immunocompetent C57BL/6 mouse model of multiple myeloma. A reproducible murine model of human multiple myeloma was developed using 5T33P cells, a derivative of the 5T33 myeloma cell line. ALT-801 significantly prolonged the survival of 5T33P myeloma bearing mice compared with PBS and re-challenged mice of the ALT-801 group survived significant longer than naïve 5T33P-instilled mice. These effects are independent of the targeting activity of the ALT-801 fusion protein and indicate that ALT-801 provide the mice with durable immunological memory response against tumors to which they were previously exposed.

As indicated above, various studies have shown that ALT-801 exhibits potent activity against HLA-A*0201⁺/p53 overexpressing (p53⁺) human melanoma, mammary adenocarcinoma, bladder cancer and pancreatic carcinoma in xenograft models in immunodeficient mice lacking T cells. Since CD8 effector T cells may contribute to the anti-tumor activity of ALT-801, additional syngeneic tumor models in immunocompetent mice were developed to further assess the efficacy of ALT-801. These tumors lack expression of the p53 peptide (aa264-272)/HLA-A*0201 complex. Thus, the effects of ALT-801 examined in these models are independent of scTCR targeting. Based on the known anti-cancer effect of immunomodulatory molecules on multiple myeloma, a murine myeloma model in immunocompetent mice was developed and used to evaluate the efficacy and mechanism of action of ALT-801.

Murine 5T33 myeloma cells, one of a series of transplantable murine myelomas arising spontaneously in C57BL/KaLwRij mice, are highly tumorigenic in C57BL/KaLwRij mice with as few as 500 cells inducing paralysis and death as early as day 36 post-tumor implantation. A 5T33-derived cell line, 5T33P, was isolated from BM of paralyzed C57BL/6 mice that had been previously instilled with 1×10⁷ of the parental 5T33 cell line. In this model, administration of at least 1×10⁷ 5 T33P cells is required to cause paralysis in C57BL/6 mice with a take rate of approximately 100%. In general, mice injected with 1×10⁷ 5 T33P cells show signs of paralysis in hind legs between SD20 and SD30 post-tumor inoculation. Besides paralysis, the expression of the 5T33-produced IgG2b paraprotein by BM cells also can be used to evaluate the tumor development status in this model.

In an initial study, the direct effects of ALT-801 on 5T33P cell growth were evaluated in vitro. Apoptosis analysis indicated that 500 nM of ALT-801 did not affect 5T33P cell proliferation and induce cellular apoptosis. Based on previous nonclinical studies, this level of ALT-801 is expected to be in the therapeutic range. Thus, ALT-801 does not appear to have a direct cytotoxic effect on the 5T33P cells.

The in vivo anti-myeloma activity of ALT-801 was then examined in immunocompetent C57BL/6 mice bearing murine 5T33P myeloma tumors. Female C57BL/6 mice (5 mice/group) were injected with 5T33P tumor cells (1×10⁷/mouse) i.v. via the lateral tail vein on study day=0 (SD0). Multidose ALT-801 treatment was then initiated 1 day (ALT-801-SD1 treatment group) or 4 days (ALT-801-SD4 treatment group) post tumor cell injection. For the ALT-801-SD1 treatment group, ALT-801 was administered i.v. at 1.6 mg/kg on SD1, SD4, SD8 and SD11 (i.e., 4 doses). 5T33P tumor-bearing mice receiving PBS (dose equivalent volume) on the same study days served as a control. For the ALT-801-SD4 treatment group, ALT-801 was administered i.v. at 1.6 mg/kg on SD4, SD8, SD11, and SD15. Mice were monitored for clinical signs of paralysis or tumor growth and survival. Mice exhibiting hind leg paralysis were considered as moribund. All of the mice of the PBS group showed signs of paralysis between SD22 and SD34 and this group had a median survival of 29 days post tumor cell administration. In contrast, all of the mice of the ALT-801-SD1 and ALT-801-SD4 groups survived beyond SD73 (the end of the observation period for the ALT-801-SD1 group), indicating that these mice were cured of the 5T33P tumors. Thus, multidose ALT-801 treatment initiated on either SD1 or SD4 was found to significantly prolong survival of 5T33P myeloma-bearing mice when compared with the PBS control group (ALT-801-SD1 vs. PBS, P<0.002; ALT-801-SD4 vs. PBS, P<0.002). No marked difference was observed between ALT-801-SD4 group and ALT-801-SD1 group (P>0.05). These results indicate that ALT-801 treatment is highly effective against 5T33P myeloma cells in this immunocompetent mouse model.

To assess whether ALT-801 treatment provides long-term anti-tumor effects, 5T33P myeloma cells were re-administered to the ALT-801-treated mice that survived previous challenge with myeloma cells. Mice of the ALT-801-SD1 treatment group (n=5) were re-challenged with 1×10⁷ 5 T33P cells on SD73 (post initial tumor cell challenge) and mice of the ALT-801-SD4 treatment group (n=5) were re-challenged with 1×10⁷ 5 T33P cells on SD106. In each case, five treatment-naïve mice were also injected with 1×10⁷ 5 T33P cells as a control for tumor development. No further ALT-801 study drug treatment was administered to any of the study groups. After tumor cell re-challenge, all five of the ALT-801-SD1 mice survived until the termination of the experiment on SD192, whereas all five of the naïve mice receiving 5T33P cells on SD73 showed paralysis between SD89 and SD107 with a median survival of 16 days post tumor cell administration. Similarly all five of the ALT-801-SD4 mice survived until SD192, whereas four of the five naïve mice receiving 5T33P cells on SD106 showed paralysis between SD124 and SD138 with a median survival of 32 days post tumor cell administration. Overall, ALT-801 treatment given over 100 days prior to 5T33P myeloma cell re-challenged significantly protected the mice from paralysis and mortality. Together, these results demonstrate not only the potency of ALT-801 against 5T33P myeloma but also its capability of inducing long-lasting immunologic memory. The above data also indicate that activated effector and memory cells of the T cell and/or NK cell subsets may play a vital role in the anti-tumor activity of ALT-801 against 5T33P tumor cells and that these immune cells probably can function for at least three months in protecting C57BL/6 mice from tumor re-challenge.

There was no observed mortality during the treatment regimen. In most cases, continuously significant body weight loss was observed just before mice exhibited hind leg paralysis in PBS and naïve treatment groups, which is a typical sign in this model. No significant body weight loss was found in ALT-801 treatment groups, which is consistent with the observation in other syngeneic mouse models using ALT-801. These findings show that the ALT-801 treatment regimens are well tolerated with transient toxicities in this model. Clinical evaluation of ALT-801 treatment in multiple myeloma should be considered in patients regardless of the patient's HLA-A*0201 genetic background.

Single dose ALT-801 treatment significantly prolonged the survival of 5T33P bearing mice compared with PBS. These effects correlated with the ability of the study drug to reduce myeloma cells in the bone marrow as assessed by an in vitro paraprotein production assay. Treatment of 5T33P tumor-bearing mice with one or two doses of ALT-801 resulted in a significant increase in the number and/or the percentage of CD8⁺ T cells and NK cells in the blood compared to the PBS group. Immune cell depletion studies demonstrated that the anti-myeloma activity of ALT-801 was primarily due to CD8+ T cells and partially due to NK cells. Other immune cells may also play a role in the ALT-801 mediated anti-myeloma effects.

The effects and functional mechanism of ALT-801 on growth of mouse 5T33P myeloma cells in C57BL/6 mouse model were further evaluated. In the first part of this study, the anti-tumor activity of single-dose ALT-801 was evaluated in this model. Female C57BL/6 mice (5 mice/group) were injected i.v. with 5T33P myeloma cells. After four days, the 5T33P tumor-bearing mice were administered a single i.v. injection of either ALT-801 (1.6 mg/kg) or PBS (dose equivalent volume). Mouse survival was monitored as the study endpoint with mice exhibiting hind leg paralysis considered as moribund. Mortality was observed in all five mice in PBS group by day 35 post tumor cell injection with a median survival of 24 days. In contrast, mortality of the ALT-801 treated mice was significantly delayed with a median survival of 49 days (vs. PBS group, P=0.013). One of 5 mice in the ALT-801 group remained alive through the 120-day observation period.

In the second part of the study, the short-term effects of single-dose ALT-801 on myeloma cells in the bone marrow were assessed in the 5T33P model. Tumor-bearing mice were treated with ALT-801 (1.6 mg/kg) or PBS and bone marrow cells were collected 1, 4 and 8 days post treatment. The cells were then cultured in vitro for 6 days and the culture supernatants were analyzed by ELISA for 5T33P cell-produced paraprotein (mouse IgG2b). ALT-801 treatment in vivo resulted in significantly lower levels of paraprotein in subsequent bone marrow cultures when compared to that of the PBS group (P<0.05). Up to a 30-fold decrease in paraprotein levels was seen in the cultures from ALT-801 treatment group. This effect was observed at all three time points of bone marrow collection post study drug treatment. Thus, the ability of single dose ALT-801 treatment to reduce bone marrow 5T33P myeloma cells (as measured by paraprotein production) was consistent with the effects of ALT-801 on prolonging survival in this model.

A further study was designed to investigate the role of effector immune cells in the anti-myeloma effects of ALT-801 against mouse 5T33P myeloma cells in immunocompetent C57BL/6 mice. Consistent with previous results in other non-clinical and clinical studies, treatment of 5T33P tumor-bearing mice with one or two doses of 1.2 mg/kg ALT-801 resulted in a significant increase in the number and/or the percentage of CD8⁺ T cells and NK cells in the blood compared to that observed in the PBS control groups. ALT-801 treatment also increased the percentage of blood CD4⁺CD25⁺FoxP3⁺ Treg cells. However, this change was significantly less than that seen with the effector CD8⁺ T cell and NK cell subsets, indicating it is not a dominant effect of ALT-801 treatment.

One or two dose treatments with 1.2 mg/kg ALT-801 was also effective at reducing the number of 5T33P myeloma cells in the bone marrow 4 days post treatment, as assessed using a bone marrow cell culture assay to detect 5T33P-derived paraprotein. Immune cell depletion studies demonstrated that the anti-myeloma activity of ALT-801 was primarily due to CD8+ T cells and partially due to NK cells. Other immune cells may play a role in the ALT-801 mediated anti-myeloma effects since the CD8+ and NK-cell depletion could not completely eliminate the anti-tumor effect on 5T33P tumor cells in the C57BL/6 mice. The results of these studies were consistent with previous studies demonstrating that ALT-801 treatment was highly effective at prolonging survival of myeloma-bearing immunocompetent mice.

### Example 9: ALT-801 Significantly Prolonged Survival of MB49luc Tumor Bearing Mice

The efficacy of intravenous administration of ALT-801 was compared to that of IL-2 in the orthotopic MB49luc muscle invasive bladder cancer model in immunocompetent C57BL/6 mice. Preclinical animal studies have indicated that ALT-801 exhibited the similar anti-tumor activity against subcutaneous HLA-A*0201⁺ p53 overexpressing (p53⁺) UMUC-14 and HLA-A*0201-negative p53 overexpressing (p53⁺) KU7 human bladder tumor xenografts in nude mice, indicating that targeting HLA-A*0201/p53 peptide complex on tumor cells seems not essential for ALT-801 therapeutic potency. Additional investigation on the effects of ALT-801 against murine MB49luc orthotopic muscle invasive bladder tumors in immunocompetent C57BL/6 mice also implied the “non-targeted” anti-tumor activity of ALT-801. It has been known that murine MB49luc tumor cells lack the human HLA-A*0201/p53 peptide complex recognized by ALT-801. Clinical studies have shown that bladder cancers exhibit modest sensitivity to IL-2-based therapies. To understand the anti-tumor activity of ALT-801, it was of interest to compare the anti-tumor activity of IL-2 and ALT-801 in bladder tumor models.

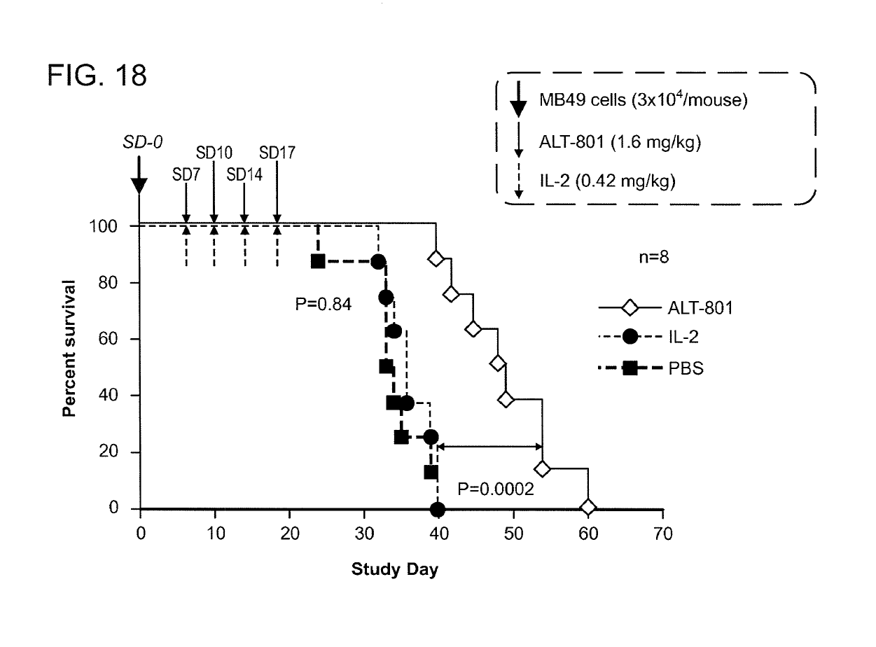

The anti-tumor effect of ALT-801 and IL-2 intravenous treatment was evaluated in a mouse bladder orthotopic model in immunocompetent C57BL/6 mice. C57BL/6 mice (10-11 weeks old) were instilled intravesically with MB49luc cells (3×10⁴ cells/bladder in 100 μL) on day 0 following polylysine pretreatment of the bladder. ALT-801 (1.6 mg/kg, n=8), IL-2 (0.42 mg/kg, n=8) or PBS (100 μL., n=8) was administered i.v. on 7, 10, 14 and 17 days post tumor cell instillation. Four intravenous doses of ALT-801 significantly prolonged the survival of mice when compared to IL-2 and PBS control (P<0.0002) (FIG. 18). No statistically significant differences were observed between the IL-2 and PBS control group (P=0.84), showing that IL-2 had no anti-tumor effect. These results indicated that twice weekly ALT-801 treatment exhibits much greater potency than recombinant human IL-2 against MB49luc bladder tumors. Similar results were also obtained from a repeated study.

### Example 10: ALT-801 Increased Survival of MB49luc Tumor Bearing Mice Following Mø, NK, or CD4 and CD8 Cell Depletion

As described above, ALT-801 treatment increased the percentage of CD3⁺ T cell, CD8⁺ T cell, and NK cell percentages in spleen and blood of MB49luc-bearing mice. In fact, blood CD8⁺ T cells remained significantly elevated throughout a four dose ALT-801 treatment course. Increased infiltration of CD3⁺ T cells and NK cell in the bladders was also observed following repeated dosing of ALT-801 in MB49luc tumor-bearing mice. In contrast, bladder macrophage levels increased with orthotopic MB49luc tumor progression regardless of ALT-801 treatment. These results indicated that one or more of these immune cell subsets play a role in the anti-tumor activity of ALT-801 in this model.

The anti-tumor effect of ALT-801 intravenous injection was evaluated following depletion of Mø, NK, or CD4 and CD8 cells in C57BL/6 mice bearing mouse MB49luc orthotopic bladder tumors. Mice received MB49luc instillation on SD 0 and then received i.v. PBS or ALT-801 (1.6 mg/kg) treatment on SD 7, 10, 14, and 17. Prior to ALT-801 or PBS treatment, groups of mice were subjected to either Mø depletion by i.p. injection of Clophosome (150 μL/dose) on SD 6, 9, 13, and 16; NK cell depletion by i.p. injection of anti-NK Ab (clone PK136, 250 μg in 100 μL) on SD 2, 3, 6, 9, 13, and 16; or CD4 and CD8 cell depletion by i.p. injection of anti-CD4 Ab (clone GK1.5, 250 μg in 100 μL) and anti-CD8 Ab (clone 53-6.72, 250 μg in 100 μL) on SD 2, 3, 6, 9, 13, and 16. Mice were maintained to assess survival rate among the study groups as the efficacy endpoint.

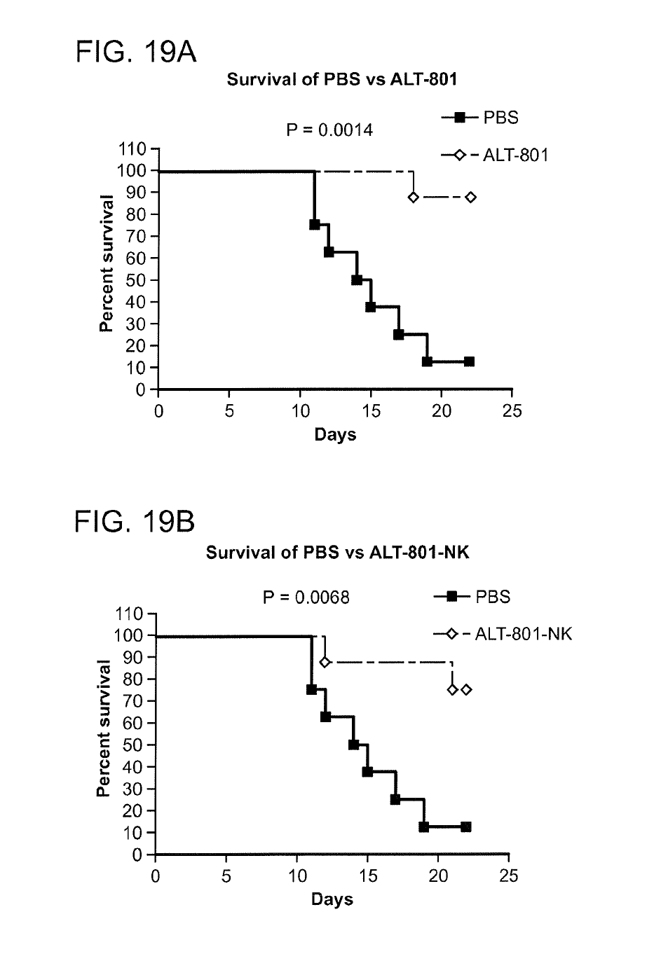

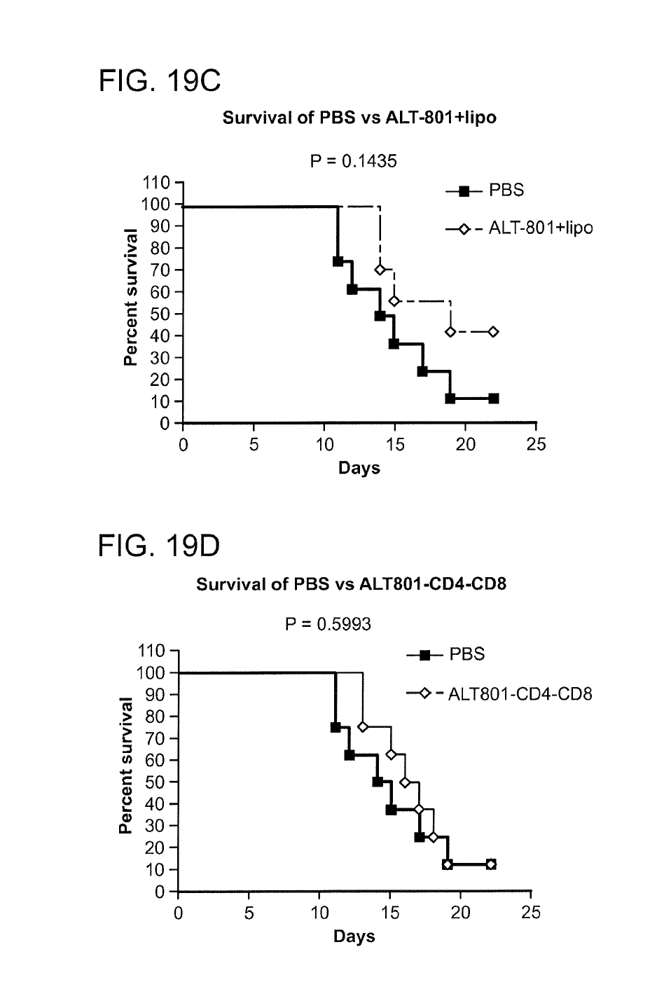

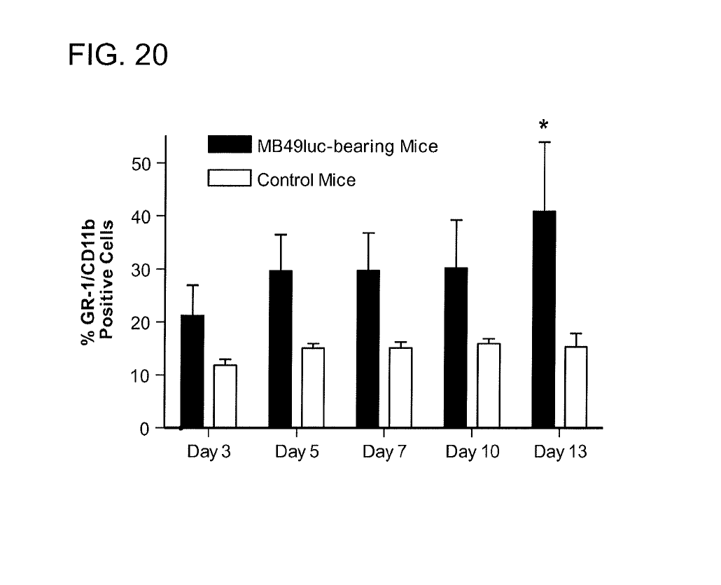

ALT-801 administered intravenously significantly prolonged the survival of mice as compared to PBS control mice (P=0.0014) (FIG. 19A). Similar results were obtained in ALT-801-treated mice which had been depleted of NK cells when compared to PBS control mice (P=0.0068) (FIG. 19B). The anti-tumor effect on survival observed following ALT-801 treatment was abrogated following depletion of Mø (P=0.1435) (FIG. 19C) or CD4/CD8 cells (P=0.5993) (FIG. 19D).

The results indicate that Mø and/or CD4/CD8 cells play an important role in the anti-tumor effects of ALT-801 in C57BL/6 mice bearing mouse MB49luc orthotopic bladder tumors. The depletion of NK cells in MB49luc tumor-bearing mice did not appear to have any effect on the efficacy of ALT-801, suggesting that NK cell are not required or that other cell types compensate for NK cell activity in ALT-801-mediated anti-tumor responses.

There is a large body of literature showing that myeloid derived suppressor cells (MDSCs) expand in a wide array of tumor models. MDSCs act to suppress NK and T cells through a variety of mechanism. Without being bound to a particular theory, the presence of MDSCs in mice bearing orthotopic MB49luc tumors may provide evidence of immunosuppressive mechanisms leading to tumor development.

To evaluate MDSC levels in this model, C57BL/6 mice were instilled intravesically with MB49luc tumor cells (0.03×10⁶ cells/mouse) as described above. Control mice did not receive tumor cells. Blood was collected from control and tumor-bearing C57BL/6 mice (5 per group) on days 3, 5, 7, 10 and 13 post tumor cell instillation. Levels of GR-1⁺/CD11b⁺ MDSCs in the blood were evaluated by flow cytometry. Blood MDSC levels were elevated in tumor bearing mice as early as 3 days post tumor cell instillation and further increased with time in these animals (FIG. 20). Blood MDSC levels in tumor bearing mice were significantly increased compared to control mice 13 days after MB49luc cell instillation.

These findings suggest that MDSCs may play a role in suppressing the immune system to promote tumor growth in the orthotopic MB49luc tumor model. This study was conducted to evaluate the roles of different immune cell types on tumor progression and the anti-tumor activity of ALT-801 in C57BL/6 mice bearing MB49luc orthotopic bladder tumors.

### Example 11: Intravenous Administration of ALT-801 Increased M1-Type Macrophages in Bladders of C57BL/6 Mice Bearing MB49luc Orthotopic Bladder Tumors

In prior pre-clinical animal studies, intravenous administration of ALT-801 prolonged survival of C57BL/6 mice bearing MB49luc orthotopic mouse bladder cancer. IHC staining of bladders from MB49luc tumor-bearing mice exhibited higher levels of CD3 and NK cell infiltration after repeated dosing with ALT-801 than were seen in bladders of PBS control treated mice. Detection of macrophage by the F4/80 pan macrophage marker indicated that more macrophages infiltrated into bladder as tumor growth progressed regardless of treatment. This study was conducted to characterize ALT-801-mediated effects on functional phenotypes of macrophages in bladders of MB49luc-bearing mice.

Macrophages play an important role in solid tumors due to their abundance, plasticity and diversity. Two distinct activation states of macrophages are recognized: the classically activated (M1) phenotype and the alternatively activated (M2) phenotype. Each type of macrophages has its own markers for identification. Features of M1 macrophages include expression of iNOS, ROS and the production of IL-12. M2 macrophages are associated with high production of IL-10, IL-1b, VEGF and matrix metalloproteinases (MMPs).

Two treatment groups, PBS and ALT-801, (3 mice/group) were included in this study. On study day (SD) 0, MB49luc cells (0.06×10⁶ cells/mouse) were instilled intravesically into bladder following 10 minutes of poly-lysine pretreatment. On SD 11, 100 μL of ALT-801 (1.6 mg/kg) or PBS were injected intravenously through the tail vein. Mice were sacrificed within 24 hours of treatment and their bladders were snap-frozen in OCT with liquid nitrogen. IHC staining was performed to check the activation states of macrophages in the bladders. iNOS and MMP-9 are used to identify M1 and M2 macrophages, respectively.

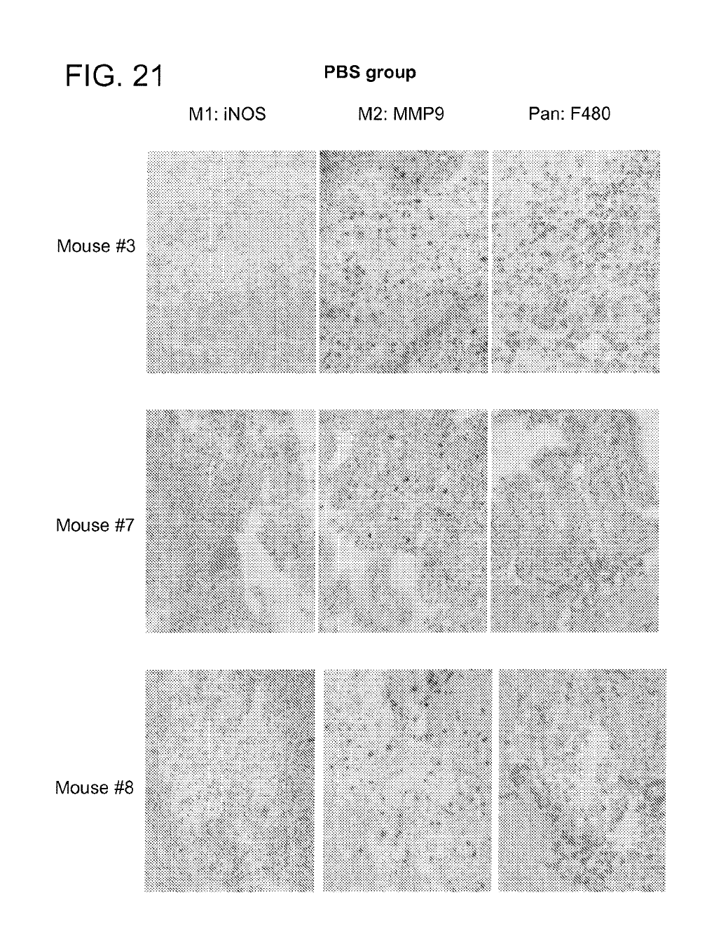

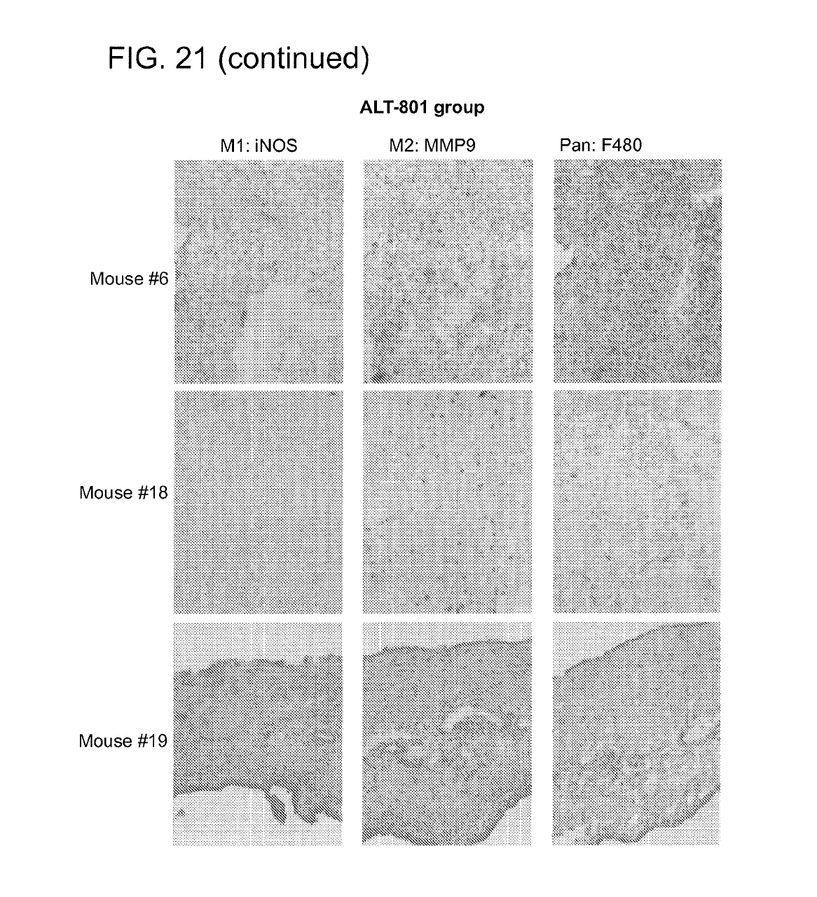

The IHC results indicated that i.v. injection of ALT-801 increased M1 type macrophages in the bladders of MB49luc tumor-bearing mice compared with bladders of the PBS treated tumor-bearing mice (FIG. 21). MMP-9 positive cells were detectable in all mice of both PBS and ALT-801 groups except one mouse in ALT-801 group. That particular mouse seemed to be tumor-free in the bladder after ALT-801 treatment and did not show any positive staining with either iNOS or MMP-9 even though F4/80 pan marker was detectable. These results indicated that macrophages were not activated in tumor-free environment since iNOS and MMP-9 are macrophage activation markers. F4/80 antibody staining showed substantial number of macrophages in the bladders of MB49luc orthotopic tumor-bearing mice compared to non-tumor bearing mice. There was no significant difference between PBS and ALT-801 treatment groups in terms of the level of F4/80 antibody staining of the bladder macrophages. In conclusion, a higher percentage of the macrophages were repolarized to the M1 phenotype in the bladders after intravenous ALT-801 treatment of MB49luc-bearing mice. Together with the finding that ALT-801 efficacy was dependent on the macrophages in MB49luc tumor-bearing mice, these results suggest that repolarized M1 macrophages may contribute to the anti-tumor effects exerted by ALT-801.

### Example 12: ALT-801 Induced IFN-γ Producing Cells in C57BL/6 Mice

Previous studies have demonstrated the anti-tumor activities of intravenous ALT-801 administration in an orthotopic MB49luc muscle invasive bladder cancer model in immunocompetent C57BL/6 mice. Mouse MB49luc cells do not express the human p53 (aa 264-272)/HLA-A*0201 complexes recognized by ALT-801. Therefore, “non-targeted” anti-tumor activity of ALT-801 against MB49luc tumor was hypothesized. The mechanism-of-action of ALT-801 against murine MB49luc bladder tumor cells was evaluated.

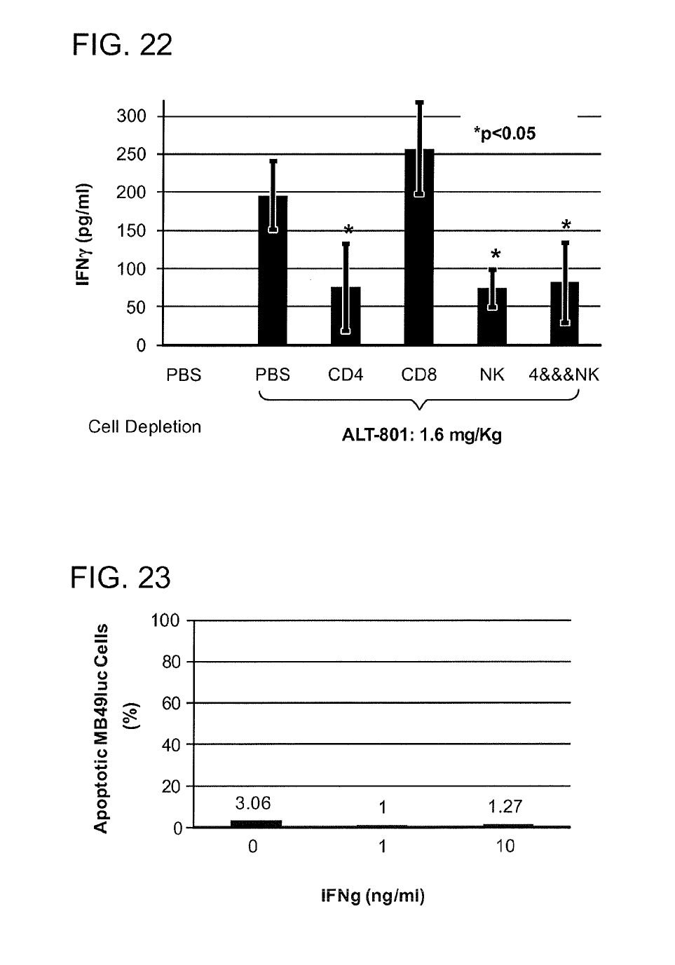

ALT-801 treatment was previously shown to increase serum levels of IFN-γ in animal models and cancer patients (Fishman et al., Clin Cancer Res, 17: 7765-7775, 2011; Wen et al., Cancer Immunol Immunother, 57: 1781-1794, 2008). IFN-γ plays an important role in anti-tumor immunity by inhibiting various tumor cell growth, up-regulating MHC molecule expression on tumor cells, activating various immune cells and anti-angiogenesis. IFN-γ can be produced by multiple subsets of immune cells, e.g. CD4⁺ T cells, CD8⁺ T cells and NK cells after activation. In this report, IFN-γ levels were assessed in the blood from C57CL/6 mice 24 hours after intravenous administration ALT-801 at 1.6 mg/kg. IFN-γ was not detectable in the serum of control mice (n=5) but reached a concentration of 196 (±44) pg/mL (n=5) after ALT-801 administration (FIG. 22). In order to investigate which cell types are the major IFN-γ producers after ALT-801 treatment, monoclonal antibodies against mouse CD4, CD8 and NK cells were injected peritoneally into C57BL/6 female mice to deplete the correspondent immune cell subsets. Serum IFN-γ levels in the immune cell-depleted mice were determined after 24 hours of ALT-801 injection. The results showed that the IFN-γ concentration in the serum of CD4, NK and triple CD4, CD8 and NK cell-depleted mice (n=5/group) reached to 75 (±58) pg/mL, 74 (±25) pg/mL and 82 (±52) pg/mL, respectively, after ALT-801 administration (FIG. 22). In contrast, serum IFN-γ levels of CD8 T cell-depleted mice (n=5) reached to 257 (±60) pg/mL. The results indicated that CD4⁺ T cells and NK cells, but not CD8⁺ T cells, are the major producers of ALT-801-induced Significant induction of serum IFN-γ could still be detected following ALT-801 treatment of the mice with triple depletion of CD4⁺, CD8⁺ T cells and NK cells. This finding indicated that other type cells besides CD4⁺ T cells and NK cells also contributed to IFN-γ production in the ALT-801-treated mice.

In the second part of this study, the effect of IFN-γ on MB49luc cell growth was investigated. MB49luc cells (2×10⁵/well) were cultured in RPMI-10 with IFN-γ at 1 or 10 ng/mL. The IFN-γ-treated MB49luc cells were harvested and stained with FITC-labeled Annexin V. The Annexin V positive apoptotic MB49luc cells were determined by flow cytometry. IFN-γ treatment does not directly result in detectable cytotoxicity against MB49luc cells (FIG. 23).

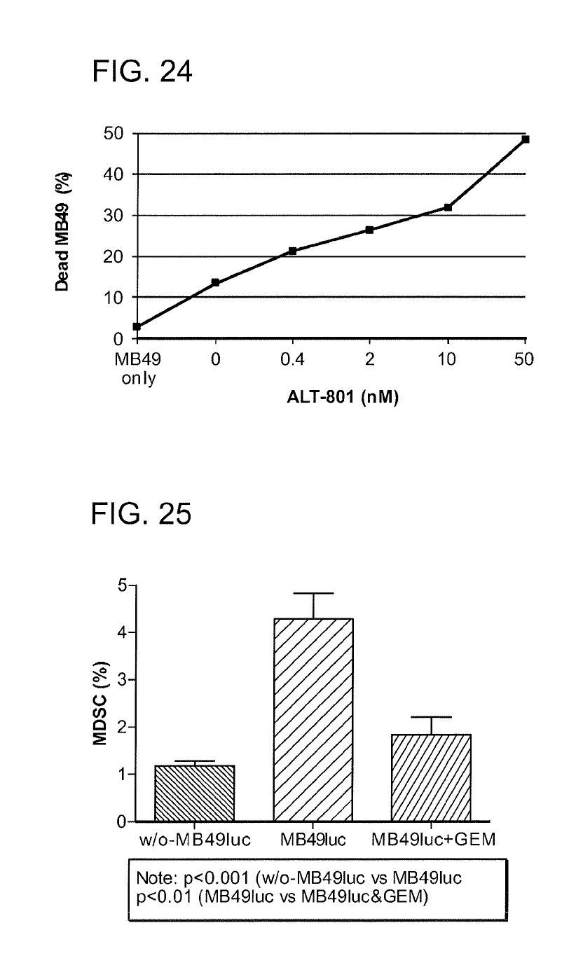

Mouse splenocytes were cultured in RPMI-10 with 20 nM ALT-801 for 3 days and then used as effector LAK cells against PKH67-labeled MB49luc target cells in cytotoxicity assays. The effector cells (4×10⁶/well) and target cells (4×10⁵/well) were incubated at 37° C. for 24 hours in RPMI-10 containing 0-50 nM ALT-801. The cytotoxicity of LAK cells against MB49luc cells was evaluated by flow cytometry based on staining with propidium iodide. ALT-801-activated splenocytes effectively lysed MB49luc cells in a manner dependent on the concentration of ALT-801 present during the cytotoxicity assay (FIG. 24).

Gemcitabine is one of the drugs in the standard combination chemotherapy for muscle invasive bladder cancer. It has been reported that gemcitabine reduces myeloid-derived suppressor cells (MDSCs) in tumor-bearing mice. In this report, we studied the effect of gemcitabine on MDSCs induced by MB49luc cells in mice. MB49luc tumor-bearing mice were treated intravenously with gemcitabine at 40 mg/kg. Three days after gemcitabine treatment, splenocytes were isolated and the percentage of Gr1⁺CD11b⁺ MDSCs was determined by flow cytometry. The MDSCs accounted for 1.19 (±0.25) percent of the splenocytes in normal control mice without MB49luc tumors. These MDSCs increased to 4.29 (±1.32) percent of splenocytes in MB49luc tumor-bearing mice. In contrast, treatment of tumor-bearing mice with gemcitabine resulted in a reduction in spleen MDSCs to 1.83 (±0.92) percent (FIG. 25). These results demonstrated that gemcitabine significantly reduced the levels of MDSCs in the spleens of MB49luc tumor-bearing mice.

It was previously shown that ALT-801 has the same activity as IL-2 to stimulate human T cells and NK cells in vitro. IL-2-activated immune cells that display cytotoxicity against various tumor cells are referred to as LAK (lymphokine-activated killer) cells. The LAK cell activity was investigated using ALT-801 pre-activated mouse splenocytes used as effector cells and MB49luc tumor cells as targets. The results of this study showed that ALT-801-activated splenocytes effectively lysed MB49luc cells in the manner that was dependent on the ALT-801 concentration during the killing phase. The finding indicates that ALT-801 is capable of activating effector immune cells and augmenting their cytotoxic activity against bladder tumor cells. Additionally, gemcitabine treatment significantly reduced the levels of MDSCs in the spleens of MB49luc tumor-bearing mice.

### Example 13: ALT-801 Induced Tumor Cell Killing by Immune Cells after MDSC Adoptive Transfer

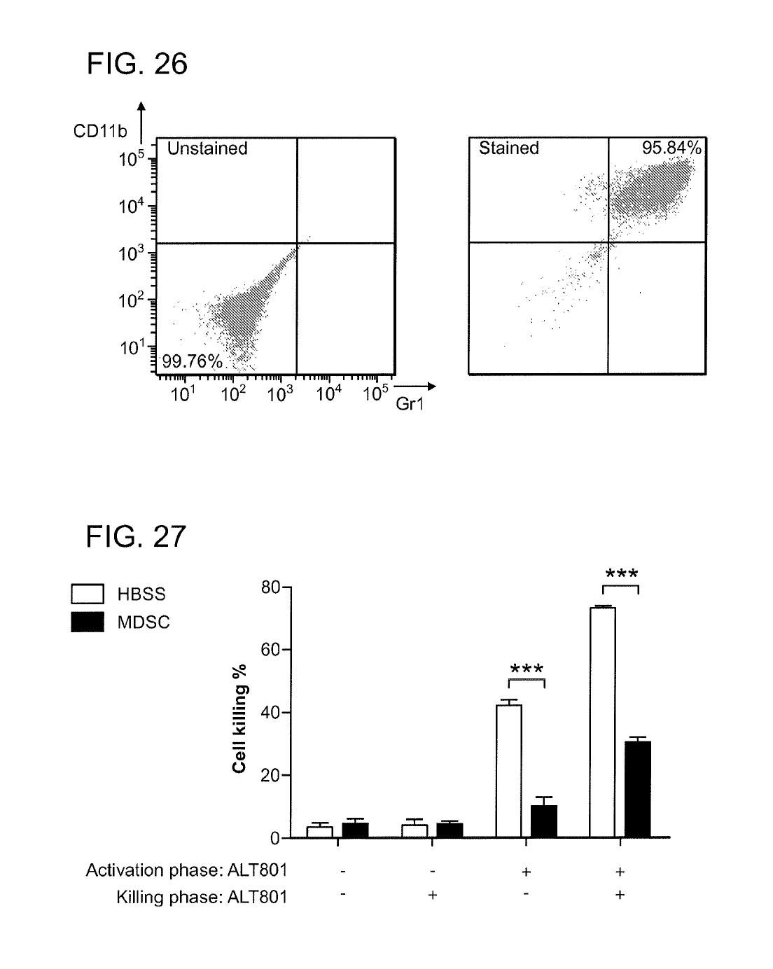

Establishment of tumors following intravenous or subcutaneous injection of MB49luc bladder tumor cells in C57BL/6 mice resulted in a significant increase in the levels of MDSCs in the blood and spleen. MDSCs are a heterogeneous population of immature myeloid cells consisting of myeloid progenitor cells, immature macrophages, immature dendritic cells, and immature granulocytes. There is a large body of literature showing that MDSCs expand in a wide array of tumor models. MDSCs act to suppress NK and T cells through direct cell contact, cytokines, and byproducts of metabolic pathways, control expansion and activation of Tregs, and support neoangiogenesis and metastatic spread of the tumor cells. In mice, MDSCs are defined by cell surface expression of CD11b⁺ and Gr1⁺. Normal mice only have a small proportion (2-4%) of spleen cells that are CD11b⁺Gr1⁺, but cells with this phenotype can reach 20-40% in some mouse tumor models. To investigate the activity of these cells, spleens were harvested from C57BL/6 mice bearing subcutaneous MB49G tumors and isolated MBSCs by magnetic sorting with anti-Gr1 and anti-Ly6G Ab beads. Through this procedure, 1×10⁷ MDSCs with 96% purity were collected from each animal (FIG. 26).

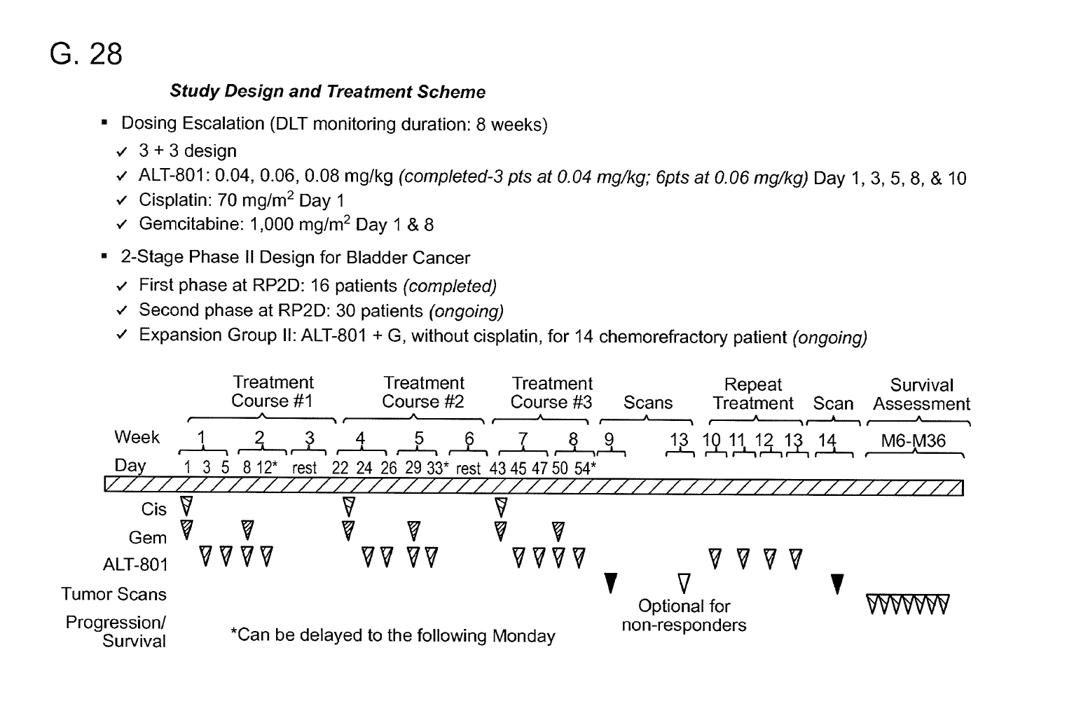

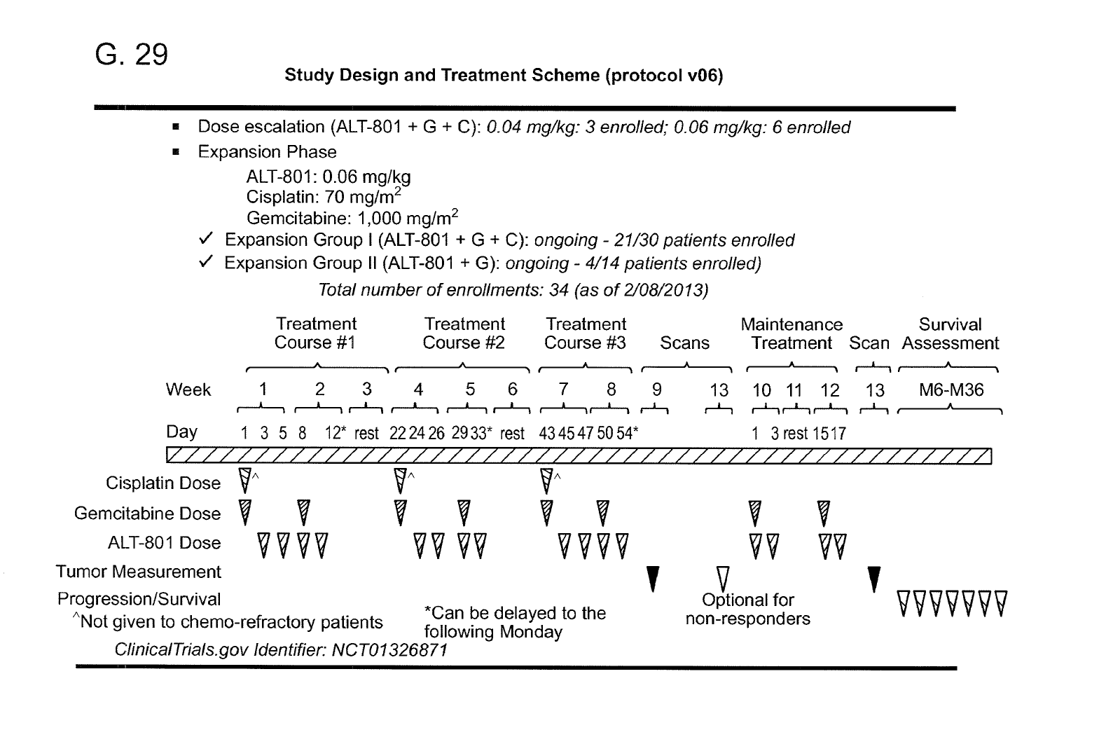

The purified MDSCs were then transferred into syngeneic normal mice to allow assessment of their immunosuppressive activity on normal immune effector cells. Forty hours after adoptive transfer, spleen cells of recipient mice were collected and activated by culturing with 50 nM ALT-801 for two days. The resulting LAK effector cells were co-cultured with PKH67-labeled MB49luc tumor cell targets overnight to assess tumor cell killing. Consistent with a previous nonclinical study on the anti-tumor effect of ALT-801, it was found that ALT-801-activated LAK cells from normal C57BL/6 mice effectively killed MB49luc tumor cells, whereas fresh splenocytes without ALT-801 activation exhibited little cytolytic activity (FIG. 27). More importantly, splenocytes isolated from mice following MDSC transfer showed significantly decreased potential as LAK cells with anti-tumor cytolytic activity following in vitro stimulation with ALT-801. Without being bound to a particular theory, these findings indicate that the presence of tumor-induced MDSCs in vivo impairs the ability of splenic effector cells to response to subsequent ALT-801 activation. Thus, the results of this study support the hypothesis that the activities of bladder tumor-induced MDSCs are detrimental to the anti-tumor effects of ALT-801.

As potent suppressors of various immune cell functions, MDSCs are potential therapeutic targets for anticancer treatment. For example, gemcitabine, a widely-used chemotherapeutic, can selectively eliminate MDSCs in tumor-bearing animals and enhance tumor-suppressive immune activity (Suzuki et al., Clin Cancer Res, 11: 6713-6721, 2005). In nonclinical studies in mouse bladder tumor models, combination therapy with gemcitabine and ALT-801 was found to be more effective than either agent as monotherapy. For example, treatment of mice bearing gemcitabine resistant subcutaneous MB49G tumors with ALT-801 (0.8 mg/kg, sub-optimal dose) in combination with gemcitabine (40 mg/kg) resulted in significantly slower tumor growth compared to that of PBS treated mice, whereas as tumor progression in mice treated with ALT-801 (0.8 mg/kg) and gemcitabine (40 mg/kg) alone did not significant differ from the PBS group. These results suggest that rather than acting directly on tumor growth, gemcitabine treatment reduces the immunosuppressive activity of MDSCs allowing ALT-801 to more effectively activate anti-tumor immune responses.

### Example 14: A Model of the Anti-Tumor Mechanism Action of ALT-801

Extensive efforts have been spent on revealing the mechanism of action of ALT-801 against cancer using various animal models, immunodepletion studies, immunohistochemistry, cytokine assays, knock-out mice, cell-mediated killing methodologies and flow cytometric analyses. Without being bound to a particular theory, the results of these research activities are consistent with the following observations:
ALT-801 activates CD4⁺ and NK cells to secrete IFN-γ.
IFN-γ activates macrophages, repolarizes tumor-associated macrophages (TAMs) from the tumor-promoting M2 to the tumor-destructive M1 stage, and induces the T_{H}1 immune responses against the tumor cells.
ALT-801 alone stimulates memory CD8⁺ T cells to proliferate and up-regulate innate-type killer receptors.
These activated CD8⁺ memory cells mount a potent, but antigen-nonspecific, cell-killing immune response against the tumors.
Both of the IFN-γ dependent pathway and the non-specific CD8⁺ memory cells are essential for anti-tumor potency of ALT-801 in vivo.

### IFN-γ and Repolarization of Tumor-Associated Macrophages

ALT-801 treatment induced secretion of IFN-γ upon infusion in normal and tumor-bearing mice. IFN-γ was at a high level both in serum and urine approximately 4-6 hrs after ALT-801 intravenous infusion (Fishman et al., Clin Cancer Res, 2011. 17:7765). CD4⁺ and NK cells are the major source of the serum IFN-γ based on an immunodepletion study showing that serum levels of IFN-γ induced by ALT-801 administration were substantially reduced by the elimination of CD4⁺ T cells and NK cells in mice (Example 12). IFN-γ did not inhibit bladder cancer cell growth nor induce apoptosis in bladder cancer cells. However, in IFN-γ Knock-Out (KO) C57BL/6 mice, ALT-801 lost its anti-bladder cancer activity against intravesically implanted MB49luc bladder tumors. Without being bound to a particular theory, immunohistochemical staining results indicated that this may be because IFN-γ is required to repolarize the M2 TAMs to M1 TAMs (Example 11). These M1 TAMs mount a rapid and potent anti-tumor response against the tumors.

IFN-γ is the most potent stimulator of monocytes and macrophages (Schroder et al., J Leukoc Biol, 2004. 75:163). The pivotal role of monocytes/macrophages in ALT-801-mediated anti-tumor activity was demonstrated by the results of studies showing that the depletion of monocytes using liposomes eliminated the efficacy of ALT-801 against orthotopic MB49luc bladder tumors (Example 10). Thus, IFN-γ (from ALT-801-activated CD4⁺ and NK cells) has the potential to activate circulating monocyte and macrophages (such as Kupffer cells in the liver) to infiltrate into the tumor lesions for cell-mediated killing of the tumors (Seki et al., Clin Dev Immunol, 2011, 2011:868345). In addition to the repolarization of TAMs and activation of monocytes and macrophages, INF-γ—a pleiotropic cytokine—is also known to exhibit various anti-tumor functions (Schroder et al., J Leukoc Biol, 2004, 75:163; Zaidi et al., Clin Cancer Res, 2011, 17:6118). It is also conceivable that INF-γ secreted from ALT-801-activated CD4⁺ and NK cells directly affects tumor growth via the activation of a large number of secondary response genes (Boehm et al., Annu Rev Immunol, 1997, 15:749).

It was found that CD4⁺ T cell depletion, but not NK cell depletion, also eliminated the anti-tumor activity of ALT-801 against MB49luc in C57BL/6 mice. ALT-801 also lost its anti-MB49luc activity in SCID mice which lack T cells. Without being bound to a particular theory, ALT-801-activated CD4⁺ T cells are capable of infiltrating the tumors and secreting IFN-γ in the tumor microenvironment to effectively re-polarize the TAMs for tumor destruction. The data of the IHC study (Example 11) are consistent with this theory.

### Memory CD8⁺ Cell-Mediated Anti-Tumor Activity Via a Novel Mechanism

In the immunodepletion study, the elimination of CD8⁺ and CD4⁺ cells, but not the NK cells alone, could eliminate the anti-tumor activity of ALT-801 in the orthotopic MB49luc bladder tumor model in CS7BL/6 mice. Thus, ALT-801-activated CD8⁺ cells are important to the anti-bladder cancer activity of ALT-801.

It has been shown recently that cytokine-mediated stimulation could promote antigen-nonspecific expansion of memory CD8⁺ cells with a unique phenotype. Unlike memory CD8⁺ T cells resulting from antigen-dependent expansion which up-regulates PD-1 and CD25, the cytokine-mediated expanded memory CD8⁺ T cells in these studies express NKG2D, granzyme B, possess broad lytic capabilities and are suggested to be responsible for the dramatic anti-tumor effects of cancer immuno-therapy (Tietze et al., Blood, 2012, 119:3073). Without being bound to a particular theory, ALT-801 activation of this type of memory CD8⁺ T cell plays a major role in the anti-MB49luc tumor activity in mice. To evaluate this possibility, it was first examined whether ALT-801 alone could induce memory CD8⁺ T cell expansion in-vitro. The phenotype of CD8⁺CD44^{high} T cells were compared after activation with ALT-801 or anti-CD3 antibody (TCR-dependent engagement). The exposure of CD8⁺ T cells to ALT-801 or anti-CD3 antibody generated CD8⁺CD44^{high} T cells with markedly different phenotypes. ALT-801 stimulation led to up-regulation of NKG2D but not higher levels of CD25 and PD-1 expression whereas anti-CD3 stimulation led to higher levels of CD25 and PD-1 expression but not NKG2D up-regulation. To examine whether a similar phenomenon occurs in-vivo, non-tumor bearing mice were injected intravenously with ALT-801 at 1.6 mg/kg (in 100 μL) or PBS (100 μL) twice (72 hours apart) and the phenotypes of PBMCs and splenocytes were analyzed one day after the second PBS or ALT-801 treatment. Levels of CD8⁺CD44^{high} memory T cells expressing NKG2D expanded following ALT-801 treatment compared levels seen in IL-2- or PBS-treated mice. In contrast, there was no up-regulation of PD-1 or CD25 by ALT-801 observed in the CD8⁺CD44^{high} memory T cell population.

Similar results were also observed in CD8⁺CD44^{high} memory T cell adoptive transfer experiments. In this study, Celltrace™ Violet-labeled splenocytes (0.5×10⁶) were adoptively transferred from naïve C57BL/6 mice into naïve isogenic C57BL/6 mice and then the mice were treated with ALT-801 or PBS intravenously one day after the adoptive cell transfer. The phenotypes of the CD8⁺CD44^{high} T cells from the spleens of the recipient mice were then analyzed one day after the second ALT-801 or PBS treatment. ALT-801, but not IL-2 or PBS, induced the proliferation of CD8⁺CD44^{high} T cells. Additionally, the NKG2D-positive cell population increased among the adoptively transferred and expanded memory CD8⁺CD44^{high} T cells in the ALT-801-treated but not in the PBS-treated recipient mice. Again, there was no up-regulation of CD25 or PD-1 on the surface of these cells observed following ALT-801 treatment. Thus, these data demonstrated that ALT-801 is apparently capable of activating CD8⁺CD44^{high} memory T cells with unique phenotype in an antigen-independent fashion.

To further demonstrate that the increased percentage of CD8⁺CD44^{high} T cells expressing the NKG2D is due to de novo-regulation of NKG2D rather than expansion of a pre-existing population of NKG2D⁺ memory CD8⁺ T cells, NKG2D⁻/CD25⁻CD8⁺/CD44^{high} T cells from naïve C57BL/6 mice were sorted. The sorted NKG2D⁻/CD25⁻/CD8⁺/CD44^{high} T cells were labeled with Celltrace™ Violet tracer, and adoptively transferred (0.4×10⁶ cells/recipient mouse) into naïve C57BL/6 mice. One day after transfer, mice were treated with two doses of PBS or with ALT-801 and splenocytes were harvested one day after the second treatment to analyze for NKG2D phenotype. NKG2D was expanded and upregulated in the Celltrace™ Violet-labeled CD8⁺CD44^{high} T cells from ALT-801-treated mice but not in PBS controls. In-vitro, the ALT-801-activated CD8⁺CD44^{high} T cells exhibited antigen-independent potent anti-tumor activity against bladder cancer cells.

Without being bound to a particular theory, the results are consistent with a model that ALT-801 activates memory CD8⁺ T cell to proliferate and up-regulate innate-like surface receptors in an antigen-independent manner. These activated memory T cells mount effective but antigen-independent killing against bladder cancer cells. It is possible that this innate-type, antigen-independent response is the reason that the anti-tumor activity is not dependent on targeting p53-peptide/HLA-A*0201 antigen

This novel mechanism of action is different from other T-cell-based immunotherapeutics, such as anti-CTLA and anti-PD-1 antibodies, for solid tumors and could enhance the potency of these studies that support these conclusions.

Designing Optimal Combination Therapies with ALT-801

Patients with cancer, especially those with advanced disease, are known to be immunologically compromised. This is because tumor cells actively induce the dysfunction of antigen presenting cells and effector cells and promote the expansion of regulatory immune cells, which down-regulate anti-tumor immunity, allowing tumor cells to escape the immune response (Whiteside, J Allergy Clin Immunol, 2010, 125:S272; Poschke et al., Cancer Immunol Immunother, 2011, 60:1161; Talmadge, Semin Cancer Biol, 2011, 21:131). The two best-characterized immunosuppressive cell subsets are FoxP3⁺ regulatory cells (Tregs) and myeloid-derived suppressor cells (MDSCs) (Qin, Cell Mol Immunol, 2009, 6:3; Gabrilovich et al., Nat Rev Immunol, 2009, 9:162; Ostrand-Rosenberg, Cancer Immunol Immunother, 2010, 59:1593.). MDSCs are a heterogeneous population of immature myeloid cells consisting of myeloid progenitor cells, immature macrophages, immature dendritic cells, and immature granulocytes (Gabrilovich et al., Nat Rev Immunol, 2009, 9:162). There is a large body of literature showing that MDSCs expand in a wide array of transplantable and autochthonous tumor models. MDSC accumulation in the blood, spleen, marrow, and tumor site is likely an early event in tumor progression due presumably to expansion and recruitment of cells from the bone marrow to the tumor site through secretion of tumor-derived factors, such as granulocyte-macrophage colony-stimulating factor and TNF-α (Bayne et al., Cancer Cell, 2012, 21:822; Pylayeva-Gupta et al., Cancer Cell, 2012, 21:836; Zhao et al., J Clin Invest, 2012, 122:4094.). MDSCs act to suppress NK and T cells through direct cell contact, cytokines, and byproducts of metabolic pathways, control expansion and activation of Tregs, promotion of Treg infiltration to the tumors, and support neoangiogenesis and metastatic spread of the tumor cells (Gabrilovich et al., Nat Rev Immunol, 2009, 9:162; Peranzoni et al., Curr Opin Immunol, 2010, 22:238; Mango et al. Immunol Rev, 2008, 222:162; Chioda et al., Cancer Metastasis Rev, 2011, 30:27; Schlecker et al., J Immunol, 2012, 189:5602).

MDSCs appear to be closely related to tumor associated macrophages (TAMs), which usually exhibit M2 polarization and can contribute to tumor progression and immune suppression by producing IL-10, TGFβ, and pro-angiogenic factors such as matrix metalloproteases, VEGF, and platelet-derived growth factor (Mantovani et al., Hum Immunol, 2009, 70:325). Recent evidence from mouse models suggests that MDSCs can differentiate into TAMs upon reaching the hypoxic environment of the tumor and thereafter display distinct phenotypic and functional characteristics (Corzo et al., J Exp Med, 2010, 207:2439).

Myeloid-Derived Suppressive Cells in Patients with Bladder Cancer:

Since the initial identification of MDSCs, several subsequent publications reported increased circulating levels of MDSCs in patients with a variety of human solid tumors (Montero et al., J Immunother, 2012, 35:107.). In patients with non-muscle invasive and muscle invasive bladder cancer, the presence of 2 distinct populations of MDSCs in the peripheral blood was reported (Eruslanov et al., Int J Cancer, 2012, 130:1109.): (i) CD11b⁺/CD15^{high}/CD33¹′ with co-expression of the neutrophil markers CD114 and CD117; and (ii) CD11b⁺/CD15^{low}/CD33^{high} with co-expression of the monocyte-macrophage markers CD14, CD115, CD116, and CCR2. When patient peripheral blood samples were compared with samples from healthy volunteers, only the CD11b⁺/CD15^{high}/CD33^{low} cells were found to be present in higher levels in bladder cancer patients, whereas the CD11b⁺/CD15^{low}/CD33^{high} cells were found to be present in significant amounts in healthy volunteers. Although both populations were found to secrete substantial amounts of cytokines, only the CD11b⁺/CD15^{high}/CD33^{low} population was noted to have immunosuppressive activity. In tumor specimens, 2 distinct MDSC populations were found to infiltrate the tumors: 60% to 70% of those cells described as CD11b⁺/HLA-DR⁺ with remaining 30% to 40% described as CD11b⁺ and CD15⁺. The clinical significance of those cells was not fully explored. In another study, a correlation was found between increased levels of circulating immunosuppressive CD14⁺/HLA-DR^{-/low} cells and clinical cancer stage and pathological grade in patients with urothelial carcinomas of the bladder. Thus, patients with urothelial carcinomas of the bladder exhibit elevated levels of MDSCs, including immunosuppressive phenotypes, which correlate with advanced disease.

Preclinical studies have been conducted that link MDSCs with bladder cancer, and are summarized below:
In the orthotopic MB49luc model in C56BL/6 mice, the intravesically implanted tumors substantially elevate the MDSCs in the blood when the disease progresses to the muscle-invasive stage (Example 10).
In this model, when the MB49luc tumor cells were implanted either subcutaneously or intravenously, similar results were observed. (Example 12).
MDSCs from MB49luc tumor-bearing C57BL/6 mice were sorted and adoptively transferred into non-tumor-bearing (recipient) C57BL/6 mice. The splenocytes from the recipient mice or wild-type C57BL/6 mice were isolated and activated in vitro by ALT-801. The cytotoxicity of ALT-801-activated splenocytes was then assessed in vitro against MB49luc cells. Splenocytes from wild-type C57BL/6 mice exhibited significantly stronger cytotoxicity against MB49luc cells than splenocytes isolated from MDSC recipient mice (Example 13). These data demonstrated the potent immune suppressive activity of MB49luc-induced MDSCs against biological activities induced by ALT-801.

The results of these studies suggest that the MDSCs induced by bladder tumor cells could hinder or interfere with the anti-tumor activity of ALT-801 in vivo.

Enhancement of ALT-801 Anti-Tumor Immune Responses by Gemcitabine:

It has been proposed that elimination of MDSCs may significantly improve antitumor responses and enhance effects of cancer immunotherapy such as ALT-801.

Gemcitabine, a major component of first-line chemotherapy for metastatic bladder cancer in humans, was found at a therapeutic dose to substantially reduce the number of MDSCs in the spleens of animals bearing large tumors without affecting the numbers of the CD4⁺ T cells, CD8⁺ T cells, NK cells, macrophages, or B cells (Suzuki et al., Clin Cancer Res, 2005, 11:6713.). The loss of MDSCs was accompanied by an increase in the anti-tumor activity of CD8⁺ T cells and NK cells. Pretreatment with gemcitabine significantly augmented antitumor effects of IFN-β on large mesothelioma tumors. In the C26 murine adenocarcinoma model, tumor-bearing mice had significantly elevated levels of MDSCs in the spleen as compared with control mice, and exhibited reduced splenocyte activation in response to IFN-α and INF-γ as measured by phosphorylation of STAT1 (Mundy-Bosse et al., Cancer Res, 2011, 71:5101.). Treatment of C26-bearing mice with gemcitabine or an anti-GR1 antibody led to depletion of MDCSs and restoration of splenocyte IFN responsiveness.

Preclinical studies have been conducted that link Gemcitabine with reduction in the activity of MDSCs induced by bladder cancer cells, and are summarized below:
In the pre-clinical MB49luc tumor model, gemcitabine treatment significantly reduced the levels of MDSCs of tumor-bearing mice (Example 12). These data suggest that gemcitabine may be a useful chemotherapeutic drug to eliminate MDSCs, thereby allowing ALT-801-stimulated immune effector cells to mediate anti-tumor activity against bladder cancer.
In the orthotopic MB49luc model in C56BL/6 mice, suboptimal levels of ALT-801 in combination with gemcitabine was as effective but exhibited less toxicity (i.e., weight loss) as ALT-801 at the same level in combination with cisplatin+gemcitabine against the MB49luc tumors. Similarly, in C57BL/6 mice bearing subcutaneous MB49luc tumors, ALT-801 in combination with gemcitabine resulted in significantly greater anti-tumor activity than either ALT-801 or gemcitabine alone.
Gemcitabine-resistant MB49luc tumor cells have been generated and used to evaluate the efficacy of a suboptimal dose of ALT-801 in combination with gemcitabine in the C57BL/6 subcutaneous tumor model. The results showed that ALT-801 at a suboptimal dose level in combination of gemcitabine exhibited significantly greater antitumor activity than either ALT-801 or gemcitabine alone.

Together these results suggest that the combination of ALT-801 and gemcitabine may provide efficacious treatment of metastatic bladder cancer, while cisplatin may be dispensable, particularly for platinum-resistant tumors. Thus, it is of interest to evaluate the anti-tumor activity of ALT-801 in combination with gemcitabine in patients with advanced bladder cancer who are refractory to platinum-based treatment. The result of this efficacy study will inform whether to remove cisplatin from the current ALT-801+gemcitabine+cisplatin regimen to treat patients with metastatic urothelial carcinomas refractory to cisplatin+gemcitabine. The non-platinum-based regimen, if proven as efficacious as the platinum-based regimen, will also greatly benefit patients who have renal insufficiency and are ineligible to receive cisplatin containing regimens. A proposal has been submitted to the U.S. FDA to enroll up to fourteen patients in an ALT-801+gemcitabine arm in the advanced bladder cancer trial, and patient enrollment for this arm began in December, 2012.

### Example 15: Human Clinical Trial Protocol

### Study Design

This is a Phase Ib/II, open-label, multi-center, competitive enrollment and dose-escalation study of ALT-801 in a biochemotherapy regimen containing cisplatin and gemcitabine in patients who have muscle invasive or metastatic urothelial cancer of bladder, renal pelvis, ureters and urethra. The study is conducted in conformity with Good Clinical Practice (GCP).

The study includes a dose escalation phase to determine the maximum tolerated dose (MTD) of ALT-801 in combination with cisplatin and gemcitabine and a two-stage expansion phase at the MTD. The dose escalation in this study is conducted using a (3+3) dose escalation design, and the two-stage expansion phase at the MTD using a modified Simon two-stage design. In the dose escalation phase of this study, there are five dose levels of ALT-801 (0.04 mg/kg, 0.06 mg/kg and 0.08 mg/kg, 0.10 mg/kg and 0.12 mg/kg) in addition to two de-escalation dose levels. The doses of cisplatin (70 mg/m²/dose) and gemcitabine (1000 mg/m²/dose) are fixed across all ALT-801 dose levels. If the MTD is not reached during the dose escalation phase, the sponsor, the Data Safety Monitoring Board and the principal investigators meet to discuss whether to amend the protocol to expand the dose escalation phase to include additional ALT-801 dose levels.

### Treatments

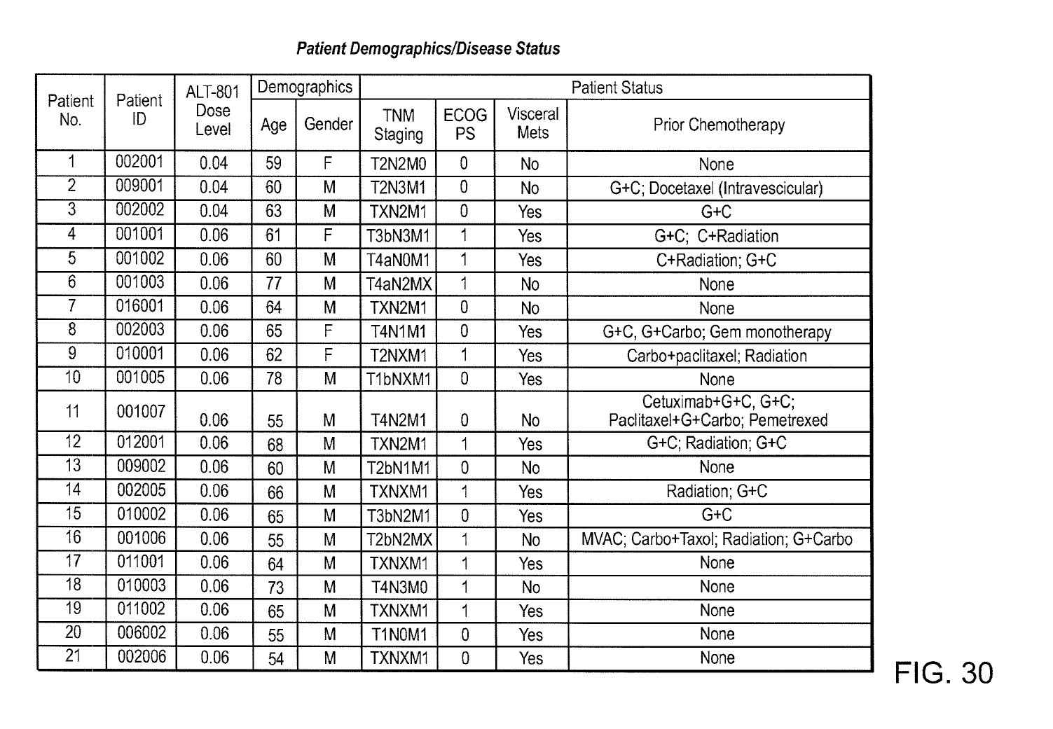

The planned initial on-study treatment is for 3 courses. Each course consists of cisplatin (Day #1), gemcitabine (Day #1), ALT-801 (Day #3 & Day #5), gemcitabine (Day #8), ALT-801 (Day #8 & Day #10), and a rest period (Days #11-21). Prior to commencing the second or the third course, subjects need to meet the continuation criteria. At the completion of the three full courses of study treatment, each patient enrolled will have been scheduled to have a total of 12 doses of the study drug ALT-801, 3 doses of cisplatin, and 6 doses of gemcitabine. After completing the 3-course initial study treatment, patients who have at least stable disease and meet other treatment criteria will repeat study treatment with four additional weekly doses of ALT-801. Delays or modifications are addressed in the protocol. This is illustrated in the following schemas and at FIGS. 28 and 29:

### Initial Study Treatment:

| | Course 1 | | | | | | Course 2 | | | | | | Course 3 | | | | | |
|---|---|---|---|---|---|---|---|---|---|---|---|---|---|---|---|---|---|---|
| | Treatment Day | | | | | | | | | | | | | | | | | |
| | 1 | 3 | 5 | 8 | 10 | 11-21 | 22 | 24 | 26 | 29 | 31 | 32-42 | 43 | 45 | 47 | 50 | 52 | 53-63 |
| Cisplatin | X | | | | | Rest | X | | | | | Rest | X | | | | | Rest |
| Gemcitabine | X | | | X | | Period | X | | | X | | Period | X | | | X | | Period |
| ALT-801 | | X | X | X | X | | | X | X | X | X | | | X | X | X | X | |

### Repeat Study Treatment:

| Dose# | 1 | 2 | 3 | 4 |
|---|---|---|---|---|
| Repeat | 1 | 8 | 15 | 22 |
| ALT-801 | X | X | X | X |

Enrolled patients receive the study treatment at qualified cancer treatment centers with adequate diagnostic and treatment facilities to provide appropriate management of therapy and complications. ALT-801, cisplatin and gemcitabine are administered by intravenous infusion into a central or peripheral vein under the supervision of a qualified physician experienced in the use of anti-cancer agents including aldesleukin (Proleukin®), cisplatin and gemcitabine. The following is the schema for the dose levels during the dose-escalation phase of the study. The −1 and −2 dose levels of ALT-801 are included in case of DLT events in the initial dose level.

| Cohort | ALT-801 Dose | Cisplatin | Gemcitabine |
|---|---|---|---|
| −2 | 0.01 | 70 | 1000 |
| −1 | 0.02 | 70 | 1000 |
| 1 | 0.04 | 70 | 1000 |
| (initial) | | | |
| 2 | 0.06 | 70 | 1000 |
| 3 | 0.08 | 70 | 1000 |
| 4 | 0.10 | 70 | 1000 |
| 5 | 0.12 | 70 | 1000 |

### Dose Escalation

In this phase of the study, a minimum of 3 patients are enrolled at each dose level. All patients are monitored for Dose Limiting Toxicity (DLT) for 8 weeks from the initial dose. If 0/3 patients have study treatment-related, dose-limiting toxicity by 8 weeks after the initial dose, the next cohort are opened for enrollment. If one patient at a dose-level develops drug-related DLT, up to six patients are enrolled at that dose level and each subsequent higher dose level. If 0 or 1 of 6 patients in a cohort of 6 patients has an event that meets criteria for study treatment-related DLT, then the next cohort are opened for enrollment. If 2 or more out of 3-6 patients in a dose escalation cohort have a DLT that is drug-related, that dose level is designated as exceeding the maximum tolerated dose. If there are 3 patients in the dose level below this level, then additional patients (up to 6 total) are enrolled at that dose level. When there is a dose level with 0 or 1 out of 6 patients with DLT, which is either the maximum planned dose level (level 5) or which is one level below a dose that was not tolerated, the dose that is the maximum tolerated dose is considered defined. Further changes in the treatment plan may be considered by protocol amendment at that point.

If more than two of six patients experience a DLT at the initial dose level (level 1), then the sponsor, the Data Safety Monitoring Board and the principal investigators meet to determine how to adjust downward the dose level of cisplatin, gemcitabine, and/or the study drug, or continue with the (−1) and (−2) cohorts, and to determine how to proceed with the study.

Dose limiting toxicity (DLT) is defined as any toxicity of grade 3 that does not resolve to Grade 1 or lower within 72 hours and any toxicity of Grade 4 occurring during treatment courses with exceptions and details described in the study protocol. Patients experiencing a DLT should discontinue study treatment. Study treatment discontinuation due to adverse events experienced prior to study drug administration, disease progression or patient's decision to withdraw from study treatment without occurrence of any study treatment discontinuation event will not necessarily define a DLT event. Study treatment discontinuation events are defined in the protocol.

### Dose Expansion

The two-stage expansion phase at the MTD are conducted using a modified Simon two-stage design. Both objective response (OR) (defined as complete response (CR)+partial response (PR)) and clinical benefit (CB) (defined as CR, PR+stable disease (SD)) are evaluated and common set thresholds of lack of efficacy (OR rate (ORR)=40%; CB rate (CBR)=78%) and an efficacy level of interest (ORR=60%; CBR=92%) are selected. The sample size is driven by the parameter that had the larger sample size for each stage.

### Stopping Rule

The patient enrollment will be temporarily suspended based on occurrence of any the following, and the sponsor, the Data Safety Monitoring Board and principal investigators will meet to discuss how to proceed with future patient enrollment in the study:
If at any time the dose escalation phase of the study, more than one patient in a cohort of three, or two of six patients experience any DLT;
If at any time during the expansion phase of the study, more than 33% the patients experience any drug related DLT.

### Evaluations

Patients are evaluated for clinical toxicities during the treatment. Patients' blood samples are collected to assess the pharmacokinetic profile and immunogenicity of the study drug. The anti-tumor response are evaluated for up to 18 weeks from the initial dose of the first course of treatment. All patients who receive at least one dose of the study drug ALT-801 are included in the anti-tumor response evaluation. Between each cohort and at the end of the study, all clinical and safety data are analyzed for all patients enrolled in the study for dose-response effects.

### Population

Patients of 18 years of age and above who are candidates for systemic cisplatin and gemcitabine for the treatment of muscle invasive or metastatic urothelial cancer of bladder, renal pelvis, ureters, and urethra may be selected for further evaluation of eligibility for study participation. Patients also need to have adequate cardiac, pulmonary, liver and kidney functions and to have an Eastern Cooperative Oncology Group (ECOG) performance status of 0 or 1 and a life expectancy of at least 12 weeks.

### Sample Size

A total of up to 30 assessable patients will be accrued to the initial dose escalation phase of the study (Phase Ib); the estimated number is 21. Up to an additional 40 assessable patients will be enrolled at the expansion phase (Stage 1 and 2) of the study (Phase II). A total of approximately 61 assessable patients will be enrolled and complete the study. Assume a 20% ineligible or non-assessable cases, a total of up 72 patients may be accrued to the study.

### Primary Endpoints

### For Stage I Only

(1) To define an MTD of ALT-801 in combination with cisplatin and gemcitabine in the treatment of patients with muscle invasive or metastatic urothelial cancer.

### For Stage I & II

(2) To assess the safety of the combination study treatment in treated patients.
(3) To assess the objective response rate in treated patients.

### Secondary Endpoints

(1) To assess the progression free survival in treated patients.
(2) To assess the overall survival in treated patients.
(3) To evaluate the immunogenicity and pharmacokinetic profiles of ALT-801 in treated patients.
(4) To assess the relationship between tumor presentation of HLA-A*0201/p53 aa 264-272 complexes and the safety and clinical benefit of study treatment.

### Pharmacokinetics & Biomarkers

Blood samples are collected to assess typing for HLA-A2, immune cell levels, phenotype, pharmacokinetics, immunogenicity of the study drug ALT-801, and the serum levels of IFN-γ and TNF-α. Tumor samples are collected to test HLA-A*0201/p53 aa 264-272 complex presentation. Blood samples for pharmacokinetic analysis of ALT-801 are taken on the first day of ALT-801 administration in the first course of study treatment. Venous blood is obtained at Time 0 (before the start of infusion), at 30 minutes (15 minutes after completion of infusion), and 1, 3 and 6 hours from Time 0 for the assessment of ALT-801 serum concentration. Non-compartmental and compartmental analyses are conducted. In addition, the same blood samples collected for PK analysis are used to assess the immunogenicity of study drug ALT-801 and the serum levels of IFN-γ and TNF-α. Fresh blood samples for HLA-A2 typing, immune cell levels and phenotype testing are collected before the start of the first and second courses of study treatment. HLA-A2 typing will be performed only once.

### Monitoring Tests

Urine samples for urinalysis, blood samples for standard chemistry, CBC, differential and coagulation are obtained at screening, on each study drug infusion day, discharge days and follow-up visits. Blood samples for immunogenicity testing, which include assays for anti-ALT-801 and IL-2 neutralizing antibodies, are collected prior to dosing on the first ALT-801 infusion day and at Week 9 from the initial dose of study treatment.

### Anti-Tumor Response Evaluation

The anti-tumor response are evaluated for up to 18 weeks from the initial dose of study treatment: for non-responders: Week 9 and 13; for early responders: Week 9 and 14; for late responders: Week 9, 13 and 18. Objective Response are evaluated using the new international criteria proposed by the Response Evaluation Criteria in Solid Tumors Committee (RECIST) 1.1. Baseline evaluations should be performed up to 28 days before starting study treatment. The same method of assessment and the same technique should be used to characterize each identified and reported lesion at baseline and during follow-ups. Imaging-based evaluation is preferred to evaluation by clinical examination when both methods have been used to assess the anti-tumor effect of the treatment. However, cystoscopic evaluation may be used routinely in this population, in addition to radiologic testing.

### Survival Assessment

Progression-free survival and overall survival of all enrolled patients are assessed at 6, 9, 12, 18, 24, 30 and 36 months from the start of study treatment, or through the point designated as the end of the study follow up.

### Adverse Events

All patients are monitored and evaluated for clinical toxicities during the treatment period and queried at each follow-up visit for Adverse Events (AEs). Patients may volunteer information concerning AEs. All adverse events are graded by using the NCI Common Terminology Criteria for Adverse Events version 4.0 (CTCAE v4.0), and logged in the patient Case Report Form. The study centers should report all SAEs and all events that trigger patient's study treatment discontinuation to the sponsor via phone, fax or email (or a combination) up to 1 day after learning of the event. The sponsor will use the information to manage and coordinate the dose escalation, cohort expansion and patient enrollment. The sponsor will then inform all of the participating clinical sites of the current dose level and the number of patients to be enrolled at that level, or of any patient enrollment suspension via phone, fax or email within a day of its learning of the event. The study centers should report the other adverse events to the sponsor following the guidelines defined in the study protocol. All study drug related adverse events (AEs) that are both serious and unexpected will be reported to the FDA in an expedited manner in accordance with 21 CFR § 312.32.

### Statistical Plan

For each cohort, all AEs are tabulated and examined and all safety and pharmacokinetic data will be evaluated. For estimation of duration of response, the Kaplan-Meier method will be used. P-values of <0.05 (two-sided) will be considered to indicate statistical significance.

### Example 16: Phase 1/2 Study for an IL-2/T-Cell Receptor Fusion Protein in Combination with Gemcitabine and Cisplatin (GC) Showed a Positive Response in Patients with Locally Advanced or Metastatic Urothelial Cancer

ALT-801 is a human IL-2/single-chain T-cell receptor fusion protein previously tested in a phase 1 in patients with advanced malignancy (Fishman et al. (2011) Clin Cancer Research 17:7765). In various murine models, ALT-801 demonstrated potent activity against syngeneic and xenograft urothelial cancer, suggesting sensitivity of this disease to IL-2 based immunotherapy (see above). Although urothelial cancers are sensitive to platinum-based chemotherapy, combinations such as gemcitabine+cisplatin are associated with complete response rates only around 15%, and limited durability of responses with limited effects of retreatment.

Methods:

Initial efficacy results of co-administration of gemcitabine (1000 mg/m²/dose, day 1 & 8), cisplatin (70 mg/m²/dose, day 1) and ALT-801 (escalating doses, days 3, 5, 8, 10) on a 21 day schedule, for 3 cycles, in patients with urothelial cancer that was locally advanced, or metastatic, for whom GC chemotherapy were considered. Patient demographics and disease status are shown at FIG. 30. ALT-801 planned doses are 0.04 to 0.12 mg/kg/dose in 5 dose cohorts with a 3+3 escalation design. Subjects with at least stable disease after 3 courses may receive 4 additional weekly doses of ALT-801 alone.

Results:

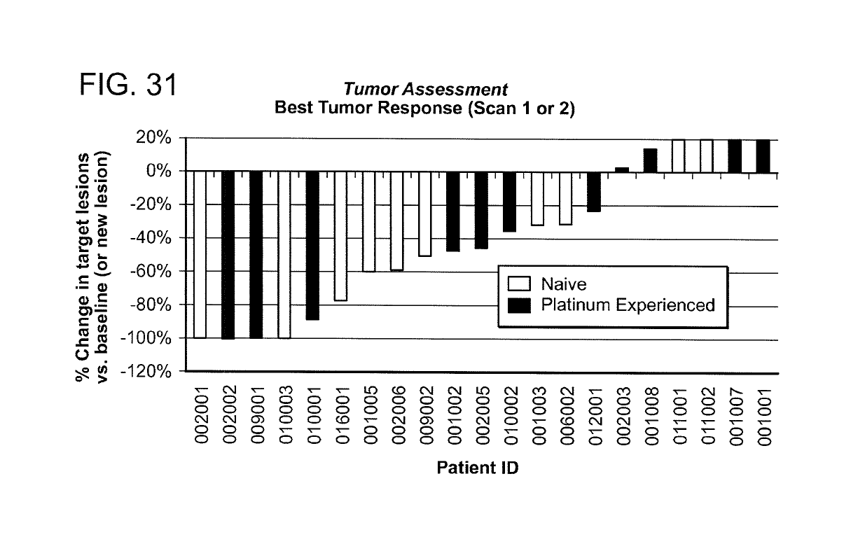

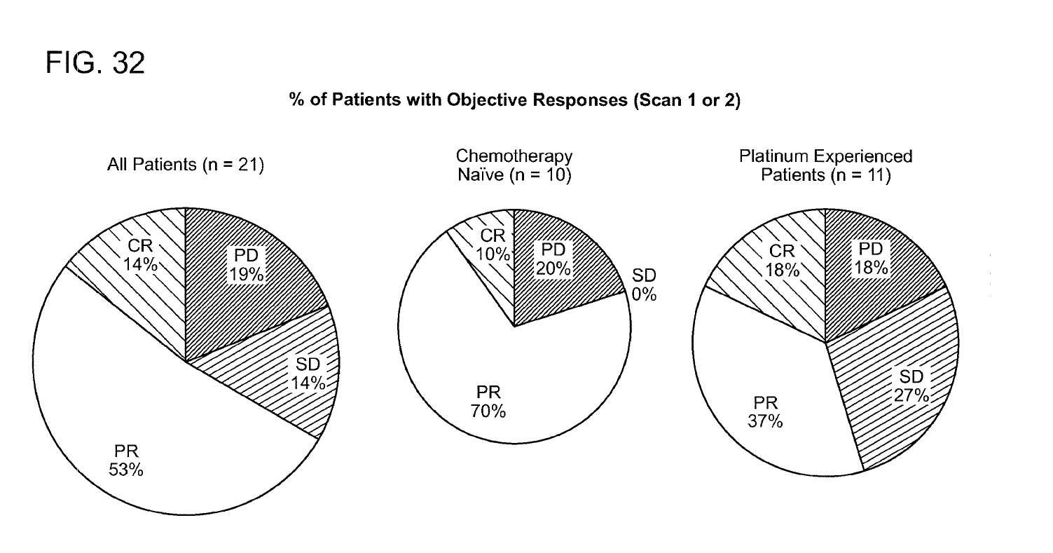

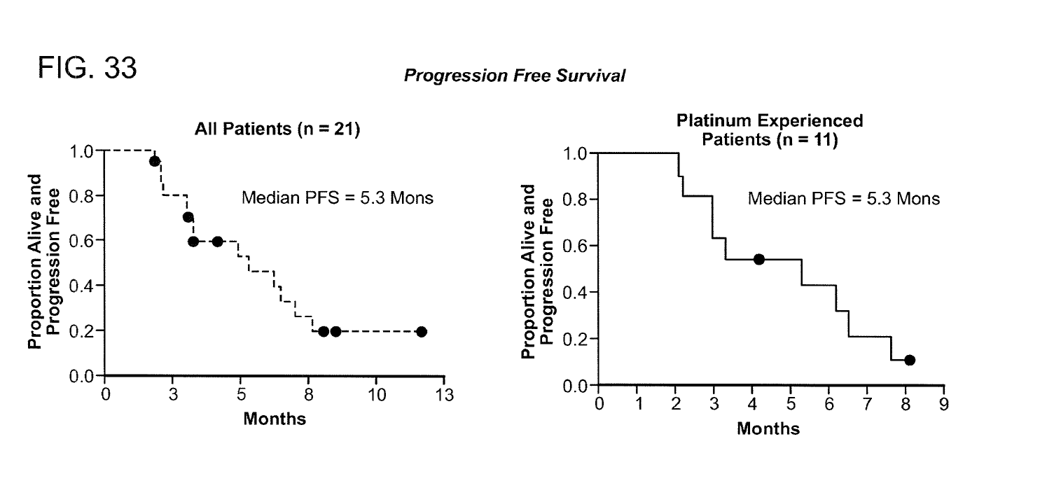

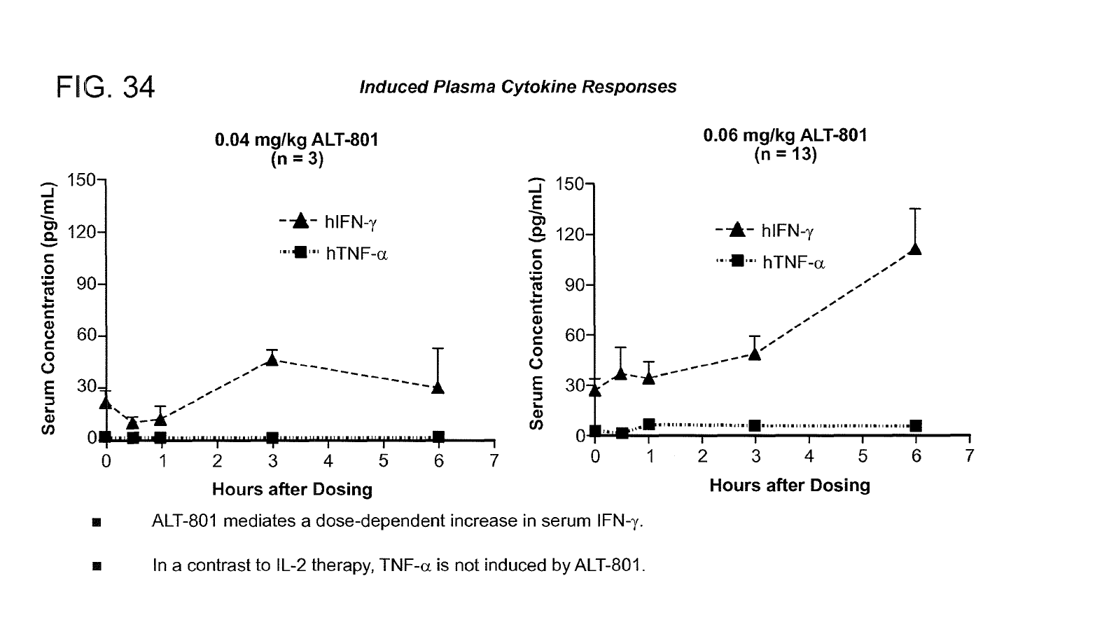

The ongoing trial of ALT-801 plus cisplatin and gemcitabine in patients with metastatic urothelial cancer is accruing well. Overall, the combination of ALT-801 plus cisplatin and gemcitabine was adequately tolerated by patients. The treatment regimen has an encouraging objective response rate (ORR) in both chemo-naïve patients and patients with chemo-refractory disease. Tumor assessment measured as percent change in target lesions showed tumor shrinkage in 71% of the patients (15 of 21) (FIG. 31). When the patients are grouped into the categories of chemotherapy naïve and platinum experienced patients, 80% of the chemotherapy naïve patients (8 of 10) and 55% of the platinum experienced patients (6 of 11) showed a positive objective response (partial or complete responses) (FIG. 32). When progression free survival is viewed, the median for all patients and platinum experienced patients was 5.3 months (FIG. 33). Presently, progression free survival was extended up to nearly 13 months in some patients compared to about 8 months in platinum experienced patients. Additionally, plasma cytokine responses were induced after administration of ALT-801 as seen by an increase in serum IFN-γ levels up to 6 hours after dosing (FIG. 34). The serum IFN-γ response was sustained at a dose of 0.06 mg/kg ALT-801 compared to a dose of 0.04 mg/kg ALT-801.

To date, at least three Stage IV urothelial cancer patients (1F, 2M; 59-63 yrs; 2 patients had predominantly nodal metastases and one patient liver metastases) have completed treatment with 0.04 mg/kg ALT-801+GC. Two had previously undergone radical cystectomy and had then later failed following GC treatment. Grade 3/4 toxicities observed include neutropenia (2), thrombocytopenia (2), leukopenia (1), lymphopenia (1) and anemia) (1), consistent with GC and ALT-801 known pharmacodynamic effects. All 3 had radiological complete responses by week 13. One patient who then underwent radical cystectomy was confirmed pathologically free of tumor cells.

The response rate (including complete responses) observed in treatment naïve subjects with advanced/metastatic urothelial cancer following treatment with ALT-801+GC is highly unexpected based on previously published clinical studies in this patient population. For example, von der Maase et al. (J. Clin. Oncol. (2000) 17:3068) reported in a Phase III clinical study of patients with advanced or metastatic bladder cancer, treatment with gemcitabine+cisplatin resulted in an overall tumor response rate (i.e., rate of partial response and complete response) of 49.4% (81 of 182 assessed patients) and a complete response rate of 12.2% by independent radiologic review. This study also reported a similar overall response rate (45.7%, 69 of 181 assessed patients) and complete response rate (11.9%) in patients treated with methotrexate, vinblastine, doxorubicin, and cisplatin. Subsequent studies of other chemotherapy regimens (i.e., single agents, doublets, triplets) in this patient population reported similar or inferior response rates (reviewed by Yafi et al. Curr. Oncol. (2011) 18:e25).

Additionally, the observed efficacy (i.e. complete and partial responses) of ALT-801+GC treatment in metastatic urothelial cancer patients who resistant to chemotherapy is also highly unexpected based on the literature. For example, no CRs were reported in a Phase III study of 370 patients with advanced urothelial cancer that progressed after a platinum-containing regimen (Bellmunt et al. J. Clin. Oncol. (2009) 27: 4454). Additionally other second line monotherapies and combination therapies for platinum-experienced patients have only provided modest effects and significant toxicities (reviewed by Yafi et al. Curr. Oncol. (2011) 18:e25).

### Other Embodiments

From the foregoing description, it will be apparent that variations and modifications may be made to the invention described herein to adopt it to various usages and conditions. Such embodiments are also within the scope of the following claims.

The recitation of a listing of elements in any definition of a variable herein includes definitions of that variable as any single element or combination (or subcombination) of listed elements. The recitation of an embodiment herein includes that embodiment as any single embodiment or in combination with any other embodiments or portions thereof. All patents and publications mentioned in this specification are herein incorporated by reference to the same extent as if each independent patent and publication was specifically and individually indicated to be incorporated by reference.

1-79. (canceled)

80. A method of treating cancer in a subject in need thereof, the method comprising:
administering to the subject an effective amount of an IL-2 fusion protein and gemcitabine to the subject in need thereof, wherein the IL-2 fusion protein comprises a T cell receptor (TCR).

81. The method of claim 80, wherein the T cell receptor is a single chain T cell receptor.

82. The method of claim 80, wherein the cancer is selected from the group consisting of bladder cancer, urothelial cancer of the urethra, ureter and renal pelvis, multiple myeloma, kidney cancer, breast cancer, colon cancer, head and neck cancer, lung cancer, prostate cancer, glioblastoma, osteosarcoma, liposarcoma, soft-tissue sarcoma, ovarian cancer, melanoma, liver cancer, esophageal cancer, pancreatic cancer and stomach cancer.

83. The method of claim 82, wherein the cancer is bladder or urothelial cancer.

84. The method of claim 80, wherein the cancer is chemo-resistant.

85. The method of claim 80, wherein the IL-2 fusion protein specifically targets p53 peptide/HLA complexes on the surface of cancer cells.

86. A method of reducing tumor burden in a subject in need thereof, the method comprising:
administering to the subject an effective amount of an IL-2 fusion protein and gemcitabine to the subject in need thereof, wherein the IL-2 fusion protein comprises a T cell receptor (TCR).

87. The method of claim 86, wherein the T cell receptor is a single chain T cell receptor.

88. The method of claim 86, wherein the tumor burden is selected from the group consisting of bladder cancer, urothelial cancer of the urethra, ureter and renal pelvis, multiple myeloma, kidney cancer, breast cancer, colon cancer, head and neck cancer, lung cancer, prostate cancer, glioblastoma, osteosarcoma, liposarcoma, soft-tissue sarcoma, ovarian cancer, melanoma, liver cancer, esophageal cancer, pancreatic cancer and stomach cancer.

89. The method of claim 88, wherein the tumor burden is bladder or urothelial cancer.

90. The method of claim 86, wherein the tumor burden is chemo-resistant.

91. The method of claim 86, wherein the IL-2 fusion protein specifically targets p53 peptide/HLA complexes on the surface of cancer cells.

92. A method of treating chemo-resistant cancer in a subject in need thereof, the method comprising:
administering to the subject an effective amount of an IL-2 fusion protein and gemcitabine to the subject in need thereof, wherein the IL-2 fusion protein comprises a T cell receptor (TCR).

93. The method of claim 92, wherein the T cell receptor is a single chain T cell receptor.

94. The method of claim 92, wherein the cancer is selected from the group consisting of bladder cancer, urothelial cancer of the urethra, ureter and renal pelvis, multiple myeloma, kidney cancer, breast cancer, colon cancer, head and neck cancer, lung cancer, prostate cancer, glioblastoma, osteosarcoma, liposarcoma, soft-tissue sarcoma, ovarian cancer, melanoma, liver cancer, esophageal cancer, pancreatic cancer and stomach cancer.

95. The method of claim 94, wherein the cancer is bladder or urothelial cancer.

96. The method of claim 92, wherein the cancer is chemo-resistant.

97. The method of claim 92, wherein the IL-2 fusion protein specifically targets p53 peptide/HLA complexes on the surface of the cancer cells.

98. The method of claim 92, wherein a durable immunological memory response against said cancer is induced, thereby inducing resistance against recurrence of said cancer.

99. A method of prolonging the survival of a subject having cancer, the method comprising:
administering to the subject an effective amount of an IL-2 fusion protein and gemcitabine to the subject in need thereof, wherein the IL-2 fusion protein comprises a T cell receptor (TCR), thereby prolonging the survival of the subject.

100. The method of claim 99, wherein the T cell receptor is a single chain T cell receptor.

101. The method of claim 99, wherein the cancer is selected from the group consisting of bladder cancer, urothelial cancer of the urethra, ureter and renal pelvis, multiple myeloma, kidney cancer, breast cancer, colon cancer, head and neck cancer, lung cancer, prostate cancer, glioblastoma, osteosarcoma, liposarcoma, soft-tissue sarcoma, ovarian cancer, melanoma, liver cancer, esophageal cancer, pancreatic cancer and stomach cancer.

102. The method of claim 101, wherein the cancer is bladder or urothelial cancer.

103. The method of claim 102, wherein the cancer is chemo-resistant.

104. The method of claim 99, wherein the IL-2 fusion protein specifically targets p53 peptide/HLA complexes on the surface of cancer cells.


SEQUENCE LISTING (12 sequences)

SEQ ID NO: 1 - protein, 552 aa, Artificial Sequence
  Description of Artificial Sequence Synthetic polypeptide
  fusion between IL-2 and a TCR domain
METDTLLLWVLLLWVPGSTGQSVTQPDARVTVSEGASLQLRCKYSYSGTPYLFWYVQYPRQGLQLLLKYYSGDPVVQGVNGFEAEFSKSNSSFHLRKASVHWSDSAVYFCVLSEDSNYQLIWGSGTKLIIKPDTSGGGGSGGGGSGGGGSGGGGSSSNSKVIQTPRYLVKGQGQKAKMRCIPEKGHPVVFWYQQNKNNEFKFLINFQNQEVLQQIDMTEKRFSAECPSNSPCSLEIQSSEAGDSALYLCASSLSGGGTEVFFGKGTRLTVVEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADVNAKTTAPSVYPLAPVSGAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 2 - protein, 532 aa, Artificial Sequence
  Description of Artificial Sequence Synthetic polypeptide
  fusion between IL-2 and a TCR domain
QSVTQPDARVTVSEGASLQLRCKYSYSGTPYLFWYVQYPRQGLQLLLKYYSGDPVVQGVNGFEAEFSKSNSSFHLRKASVHWSDSAVYFCVLSEDSNYQLIWGSGTKLIIKPDTSGGGGSGGGGSGGGGSGGGGSSSNSKVIQTPRYLVKGQGQKAKMRCIPEKGHPVVFWYQQNKNNEFKFLINFQNQEVLQQIDMTEKRFSAECPSNSPCSLEIQSSEAGDSALYLCASSLSGGGTEVFFGKGTRLTVVEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADVNAKTTAPSVYPLAPVSGAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 3 - DNA, 1659 bp, Artificial Sequence
  Description of Artificial Sequence Synthetic polynucleotide
  fusion between IL-2 and a TCR domain
atggagacagacacactcctgttatgggtactgctgctctgggttccaggttccaccggtcagtcagtgacgcagcccgatgctcgcgtcactgtctctgaaggagcctctctgcagctgagatgcaagtattcctactctgggacaccttatctgttctggtatgtccagtacccgcggcaggggctgcagctgctcctcaagtactattcaggagacccagtggttcaaggagtgaatggcttcgaggctgagttcagcaagagtaactcttccttccacctgcggaaagcctctgtgcactggagcgactctgctgtgtacttctgtgttttgagcgaggatagcaactatcagttgatctggggctctgggaccaagctaattataaagccagacactagtggtggcggtggcagcggcggtggtggttccggtggcggcggttctggcggtggcggttcctcgagcaattcaaaagtcattcagactccaagatatctggtgaaagggcaaggacaaaaagcaaagatgaggtgtatccctgaaaagggacatccagttgtattctggtatcaacaaaataagaacaatgagtttaaatttttgattaactttcagaatcaagaagttcttcagcaaatagacatgactgaaaaacgattctctgctgagtgtccttcaaactcaccttgcagcctagaaattcagtcctctgaggcaggagactcagcactgtacctctgtgccagcagtctgtcagggggcggcacagaagttttctttggtaaaggaaccagactcacagttgtagaggacctgaacaaggtgttcccacccgaggtcgctgtgtttgagccatcagaagcagagatctcccacacccaaaaggccacactggtgtgcctggccacaggcttcttccctgaccacgtggagctgagctggtgggtgaatgggaaggaggtgcacagtggggtcagcacggacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacccgtcacccagatcgtcagcgccgaggcctggggtagagcagacgttaacgcaaagacaaccgccccttcagtatatccactagcgcccgtttccggagcacctacttcaagttctacaaagaaaacacagctacaactggagcatttactgctggatttacagatgattttgaatggaattaataattacaagaatcccaaactcaccaggatgctcacatttaagttttacatgcccaagaaggccacagaactgaaacatcttcagtgtctagaagaagaactcaaacctctggaggaagtgctaaatttagctcaaagcaaaaactttcacttaagacccagggacttaatcagcaatatcaacgtaatagttctggaactaaagggatctgaaacaacattcatgtgtgaatatgctgatgagacagcaaccattgtagaatttctgaacagatggattaccttttgtcaaagcatcatctcaacactaacttaa

SEQ ID NO: 4 - protein, 548 aa, Artificial Sequence [sequence omitted]

SEQ ID NO: 5 - protein, 528 aa, Artificial Sequence
  Description of Artificial Sequence Synthetic polypeptide
  fusion between IL-2 and a TCR domain
QKEVEQNSGPLSVPEGAIASLNCTYSDRGSQSFFWYRQYSGKSPELIMFIYSNGDKEDGRFTAQLNKASQYVSLLIRDSQPSDSATYLCAVNFGGGKLIFGQGTELSVKPDTSGGGGSGGGASGGGGSGGGGSSSIAGITQAPTSQILAAGRRMTLRCTQDMRHNAMYWYRQDLGLGLRLIHYSNTAGTTGKGEVPDGYSVSRANTDDFPLTLASAVPSQTSVYFCASSLSFGTEAFFGQGTRLTVVEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADVNAKTTAPSVYPLAPVSGAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 6 - DNA, 1646 bp, Artificial Sequence
  Description of Artificial Sequence Synthetic polynucleotide
  fusion between IL-2 and a TCR domain
atggagacagacacactcctgttatgggtactgctgctctgggttccaggttccaccggtcagaaggaggtggagcagaattctggacccctcagtgttccagagggagccattgcctctctcaactgcacttacagtgaccgaggttcccagtccttcttctggtacagacaatattctgggaaaagccctgagttgataatgttcatatactccaatggtgacaaagaagatggaaggtttacagcacagctcaataaagccagccagtatgtttctctgctcatcagagactcccagcccagtgattcagccacctacctctgtgccgtgaacttcggaggaggaaagcttatcttcggacagggaacggagttatctgtgaaacccgacactagtggtgggggtgggagcgggggtggtgctagcggtggcggcggttctggcggtggcggttcctccagcattgcagggatcacccaggcaccaacatctcagatcctggcagcaggacggcgcatgacactgagatgtacccaggatatgagacataatgccatgtactggtatagacaagatctaggactggggctaaggctcatccattattcaaatactgcaggtaccactggcaaaggagaagtccctgatggttatagtgtctccagagcaaacacagatgatttccccctcacgttggcgtctgctgtaccctctcagacatctgtgtacttctgtgccagcagcctaagtttcggcactgaagctttctttggacaaggcaccagactcacagttgtagaggacctgaacaaggtgttcccacccgaggtcgctgtgtttgagccatcagaagcagagatctcccacacccaaaaggccacactggtgtgcctggccacaggcttcttccctgaccacgtggagctgagctggtgggtgaatgggaaggaggtgcacagtggggtcagcacggacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacccgtcacccagatcgtcagcgccgaggcctggggtagagcagacgttaacgcaaagacaaccgccccttcagtatatccactagcgcccgtttccggagcacctacttcaagttctacaaagaaaacacagctacaactggagcatttactgctggatttacagatgattttgaatggaattaataattacaagaatcccaaactcaccaggatgctcacatttaagttttacatgcccaagaaggccacagaactgaaacatcttcagtgtctagaagaagaactcaaacctctggaggaagtgctaaatttagctcaaagcaaaaactttcacttaagacccagggacttaatcagcaatatcaacgtaatagttctggaactaaagggatctgaaacaacattcatgtgtgaatatgctgatgagacagcaaccattgtagaatttctgaacagatggattaccttttgtcaaagcatcatctcaacactaactta

SEQ ID NO: 7 - protein, 9 aa, Homo sapiens
LLGRNSFEV

SEQ ID NO: 8 - protein, 9 aa, Artificial Sequence
  Description of Artificial Sequence Synthetic linker peptide
SGGGGSGGG

SEQ ID NO: 9 - protein, 23 aa, Artificial Sequence
  Description of Artificial Sequence Synthetic linker peptide
TSGGGGSGGGGSGGGGSGGGGSS

SEQ ID NO: 10 - protein, 18 aa, Artificial Sequence
  Description of Artificial Sequence Synthetic linker peptide
VNAKTTAPSVYPLAPVSQ

SEQ ID NO: 11 - protein, 5 aa, Artificial Sequence
  Description of Artificial Sequence Synthetic peptide
  MOD_RES (1)..(1): N,N-dimethyl-L-valyl
  MOD_RES (2)..(2): L-valyl
  MOD_RES (3)..(3): N-methyl-L-valyl
  MOD_RES (4)..(4): L-prolyl
  MOD_RES (5)..(5): Lproline-t-butylamide
VVVPP

SEQ ID NO: 12 - protein, 6 aa, Artificial Sequence
  Description of Artificial Sequence Synthetic 6xHis tag
HHHHHH